(19) United States
(12) Patent Application Publication
(10) Pub. No.: US 2008/0194552 A1
(43) Pub. Date: Aug. 14, 2008

(54) Aminopyrimidine Derivatives With Tie2 Inhibiting Activity

(75) Inventors: Clifford David Jones, Cheshire (GB); Richard William, Arthur Luke, Cheshire (GB); William McCoull, Cheshire (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 11/910,071

(22) PCT Filed: Mar. 30, 2006
(86) PCT No.: PCT/GB2006/001175

§ 371 Date: Sep. 28, 2007

(30) Foreign Application Priority Data
Mar. 31, 2005 (GB) 0506467.0
Jun. 21, 2005 (GB) 0512611.5
Jun. 21, 2005 (GB) 0512615.6

(51) Int. Cl. A61K 31/5377; C07D 409/06; A61K 31/496; A61P 35/04; C07D 413/14

(52) U.S. Cl. 514/232.2; 544/297; 514/275; 544/122; 514/252.14

(57) ABSTRACT

The invention relates to a compound of the Formula I or salt thereof wherein R^{x}, R^{y}, R^{x}, R⁵, R⁶, A, B, L, n and m are as defined in the description. The invention also relates to pharmaceutical compositions of said compounds, the use of said compounds as medicaments and in the production of an anti-angiogenic effect in a warm blooded animal. The invention also relates to processes for the preparation of said compounds.

This invention relates to compounds, or pharmaceutically acceptable salts thereof, which possess anti-angiogenic activity and are accordingly useful in methods of treatment of disease states associated with angiogenesis in the animal or human body. The invention also concerns processes for the preparation of the compounds, pharmaceutical compositions containing the compounds as active ingredient, and methods for the use of the compounds in the manufacture of medicaments for use in the production of anti-angiogenic effects in warm-blooded animals such as humans.

The Tie2 receptor tyrosine kinase (also known as TEK) is expressed predominantly in endothelial and haematopoietic cells and is essential for vessel formation and maintenance (Jones, N. et al. Nature Reviews Molecular Cell Biology. 2001: 2, 257-67).

Angiogenesis is a fundamental process defined as the generation of new blood vessels from existing vasculature. It is a vital yet complex biological process required for the formation and physiological functions of virtually all the organs. Normally it is transient in nature and is controlled by the local balance of angiogenic and angiostatic factors in a multi-step process involving vessel sprouting, branching and tubule formation by endothelial cells (involving processes such as activation of endothelial cells (ECs), vessel destabilisation, synthesis and release of degradative enzymes, EC migration, EC proliferation, EC organisation and differentiation and vessel maturation).

Normal angiogenesis plays an important role in a variety of processes and is under stringent control. In the adult, physiological angiogenesis is largely confined to wound healing and several components of female reproductive function and embryonic development. In undesirable or pathological angiogenesis, the local balance between angiogenic and angiostatic factors is dysregulated leading to inappropriate and/or structurally abnormal blood vessel formation. Pathological angiogenesis has been associated with disease states including diabetic retinopathy, psoriasis, cancer, rheumatoid arthritis, atheroma, Kaposi's sarcoma and haemangioma (Fan et al, 1995, Trends Pharmacology. Science. 16: 57-66; Folkman, 1995, Nature Medicine 1: 27-31). In cancer, growth of primary and secondary tumours beyond 1-2 mm³ requires angiogenesis (Folkman, J. New England Journal of Medicine 1995; 33, 1757-1763).

In diseases such as cancer in which progression is dependant on aberrant angiogenesis, blocking the process can lead to prevention of disease advancement (Folkman, J. 1995, Nature Medicine. 1: 27-31). Many factors are described in the scientific literature that are believed to play important critical roles in the regulation of angiogenesis. Two major classes of angiogenic factors are the vascular endothelial growth factor (VEGF) and the angiopoietins. These polypeptide moieties interact with their respective receptors (transmembrane tyrosine kinases which are predominantly endothelial cell specific) and induce cellular responses via ligand mediated signal transduction. It has been speculated that VEGF and the angiopoietins co-operate to regulate various aspects of the angiogenic process during both normal and pathological angiogenesis via signalling through their respective receptors.

Receptor tyrosine kinases (RTKs) are important in the transmission of biochemical signals across the plasma membrane of cells. These transmembrane molecules characteristically consist of an extracellular ligand-binding domain connected through a segment in the plasma membrane to an intracellular tyrosine kinase domain. Binding of ligand to the receptor results in stimulation of the receptor-associated tyrosine kinase activity that leads to phosphorylation of tyrosine residues on both the receptor and other intracellular molecules. These changes in tyrosine phosphorylation initiate a signalling cascade leading to a variety of cellular responses. To date, at least nineteen distinct RTK subfamilies, defined by amino acid sequence homology, have been identified. One of these subfamilies is presently comprised by the fins-like tyrosine kinase receptor, Flt or Flt1, the kinase insert domain-containing receptor, KDR (also referred to as Flk-1), and another fins-like tyrosine kinase receptor, Flt4. Two of these related RTKs, Flt and KDR, have been shown to bind VEGF with high affinity (De Vries et al, 1992, Science 255: 989-991; Terman et al, 1992, Biochem. Biophys. Res. Comm. 1992, 187: 1579-1586). Binding of VEGF to these receptors expressed in heterologous cells has been associated with changes in the tyrosine phosphorylation status of cellular proteins and calcium fluxes.

Recently a second family of predominantly endothelial cell specific receptors that regulate vessel destabilisation and maturation have been identified. The Tie receptors and their ligands, the angiopoietins, co-operate closely with VEGF during both normal and pathological angiogenesis. The transmembrane receptors Tie1 and Tie2, constitute a family of endothelial cell specific tyrosine kinase receptors involved in maintenance of blood vessel integrity and which are involved in angiogenic outgrowth and vessel remodelling. Structurally Tie1 and Tie2 share a number of features (e.g. the intracellular domains of both these receptors each contain a tyrosine kinase domain interrupted by a kinase insert region) and thus constitute a distinct RTK subfamily. Overall sequence identity between Tie1 and Tie2 receptors at the amino acid level is 44% while their intracellular domains exhibit 76% homology. Targeted disruption of the Tie l gene results in a lethal phenotype characterised by extensive haemorrhage and poor microvessel integrity (Puri, M. et al. 1995 EMBO Journal: 14:5884-5891). Transgenic mice deficient in Tie2 display defects in vessel sprouting and remodelling and display a lethal phenotype in mid gestation (E9.5-10.5) caused by severe defects in embryonic vasculature (Sato, T. et al. 1995 Nature 370: 70-74).

To date no ligands have been identified for Tie1 and little is known regarding its signalling abilities. However, Tie1 is believed to influence Tie2 signalling via heterodimerisation with the Tie2 receptor, hence potentially modulating the ability of Tie2 to autophosphorylate (Marron, M. et al. 2000 Journal of Biological Chemistry: 275, 39741-39746) and recent chimaeric Tie1 receptor studies have indicated that Tie-1 may inhibit apoptosis via the PI 3 kinase/Akt signal transduction pathway (Kontos, C. D., et al., 2002 Molecular and Cellular Biology: 22, 1704-1713). In contrast, a number of ligands, designated the angiopoietins have been identified for Tie2 of which Angiopoietin 1 (Ang1) is the best characterised. Binding of Ang1 induces tyrosine phosphorylation of the Tie2 receptor via autophosphorylation and subsequently activation of its signalling pathways via signal transduction. Ang2 has been reported to antagonise these effects in endothelial cells (Maisonpierre, P. et al. 1997 Science: 277, 55-60). The knock-out and transgenic manipulation of Tie2 and its ligands suggest that stringent spatial and temporal control of Tie2 signalling is imperative for the correct development of new vasculature. There are also reports of at least another two ligands (Ang3 and Ang4) as well as the possibility of heterodimerisation between the angiopoietin ligands that has the potential to modify their activity (agonistic/antagonistic) on association with the receptor. Activation of the Tie2 receptor by Ang1 inhibits apoptosis (Papapetropoulos, A., et al., 2000 Journal of Biological Chemistry: 275 9102-9105), promotes sprouting in vascular endothelial cells (Witzenbicher, B., et al., 1998 Journal of Biological Chemistry: 273, 18514-18521) and in vivo promotes blood vessel maturation during angiogenesis and reduces the permeability and consequent leakage from adult microvessels (Thurston, G. et al., 2000 Nature Medicine: 6, 460-463). Thus activated Tie2 receptor is reported to be involved in the branching, sprouting and outgrowth of new vessels and recruitment and interaction of periendothelial support cells important in maintaining vessel integrity and overall appears to be consistent with promoting microvessel stability. Absence of Tie2 activation or inhibition of Tie2 auto phosphorylation may lead to a loss of vascular structure and matrix/cell contacts (Thurston, G., Cell Tissue Res (2003), 314: 61-69) and in turn may trigger endothelial cell death, especially in the absence of survival or growth stimuli. On the basis of the above reported effects due to Tie2 kinase activity, inhibiting Tie2 kinase may provide an anti-angiogenic effect and thus have application in the therapy of disease states associated with pathological angiogenesis. Tie2 expression has been shown to be up-regulated in the neovasculature of a variety of tumours (e.g. Peters, K. G. et al, (British Journal of Cancer, 1998; 77,51-56) suggesting that inhibiting Tie2 kinase activity will result in anti-angiogenic activity. In support of this hypothesis, studies with soluble Tie2 receptor (extracellular domain) (Pengnian, L. et al., 1997, Journal of Clinical Investigation 1997: 100, 2072-2078 and Pengnian, L. et al., 1998, Proceedings of the National Academy of Sciences 1998: 95, 8829-8834) have shown anti-tumour activity in in vivo tumour models. In addition these experiments also indicate that disruption of the Tie2 signalling pathways in a normal healthy individual may be well tolerated as no adverse toxicities were observed in these studies.

Examination of human primary breast cancer samples and human and murine breast cancer cell lines (Stratmann, A., et al., 2001, International Journal of Cancer: 91, 273-282) indicate that Tie2 dependant pathways of tumour angiogenesis may exist alongside KDR dependant pathways and, in fact, may operate both independently (Siemeister G., et al., 1999 Cancer Research: 59, 3185-3191) as well as in concert with each other (e.g. VEGF A and Angl reported to collaborate to induce angiogenesis and produce non-leaky mature vessels Thurston, G, et al., 1999 Science: 286, 2511-2514). It is quite possible that a mix of such angiogenic processes even exist within a single tumour.

Tie2 has also been shown to play a role in the vascular abnormality called venous malformation (VM) (Mulliken, J. B. & Young, A. E. 1998, Vascular Birthmarks: W. B. Saunders, Philadelphia). Such defects can either be inherited or can arise sporadically. VM's are commonly found in the skin or mucosal membranes but can affect any organ. Typically lesions appear as spongy, blue to purple vascular masses composed of numerous dilated vascular channels lined by endothelial cells. Among the inherited forms of this disease the most common defect appears to be a Tie2 kinase mutation C2545T in the Tie2 coding sequence (Calvert, J. T., et al., 1999 Human Molecular genetics: 8, 1279-1289), which produces a R849W amino acid substitution in the kinase domain. Analysis of this Tie2 mutant indicates that it is constitutively activated even in the absence of ligand (Viklula, M., et al., 1996 Cell: 87, 1181-1190).

Upregulation of Tie2 expression has also been found within the vascular synovial pannus of arthritic joints in humans, which is consistent with the role of inappropriate neovascularisation.

Such examples provide further indications that inhibition of Tie2 phosphorylation and subsequent signal transduction will be useful in treating disorders and other occurrences of inappropriate neovascularisation. To date only a few inhibitors of Tie2 are known in the art. For example, Internation Application No: WO 04/013141 describes a group of condensed pyridines and pyrimidines and International Application No: WO 04/058776 describes a group of pryidine and pyrimidine compounds. There is thus a need to identify additional Tie2 inhibitors that could exploit the full therapeutic potential of inhibiting/ modulating the Tie2 signalling pathways.

We have found that certain compounds possess inhibitory activity for the Tie2 receptor tyrosine kinase and accordingly have value in the treatment of disease states associated with pathological angiogenesis such as cancer, rheumatoid arthritis, and other diseases where active angiogenesis is undesirable.

According to the present invention there is provided a compound of Formula I:

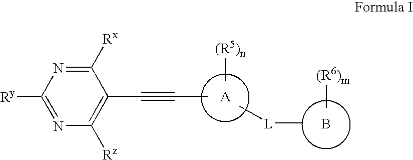

wherein one of R^{x} or R^{y} is a group NR¹R², and the other is a group R³ or R⁴, and R^{z} is a group R³ or R⁴,
R¹ and R² are independently selected from hydrogen, (1-6C)alkylsulfonyl, phenyl(CH₂)ₙ— wherein u is 0, 1, 2, 3, 4, 5 or 6, (1-6C)alkanoyl, (1-6C)alkyl, (1-6C)alkoxycarbonyl, (3-6C)cycloalkyl(CH₂)ᵥ— in which v is 0, 1, 2, 3, 4, 5 or 6, or a 5 or 6 membered heteroaryl ring, or R¹ and R² together with the nitrogen atom to which they are attached represent a saturated or partially saturated 3 to 7 membered heterocyclic ring optionally containing another heteroatom selected from N or O;

wherein a (1-6C)alkyl, the (1-6C)alkoxy, the (1-6C)alkanoyl and the (3-6C)cycloalkyl groups are optionally substituted by one or more groups independently selected from fluoro, hydroxy, (1-6C)alkyl, (1-6C)alkoxy, (1-6C)alkoxy(1-6C)alkoxy, (1-6C)alkoxy(1-6C)alkoxy(1-6C)alkoxy, amino, mono(1-6C)alkylamino, di-[(1-6C)alkyl]amino, carbamoyl, mono(1-6C)alkylcarbamoyl, di-[(1-6C)alkyl]carbamoyl or —N(R^{d})C(O)(1-6C)alkyl, —N(R^{d})C(O)(1-6C)alkyl in which R^{d} is hydrogen or (1-6C)alkyl, or a saturated or partially saturated 3 to 7 membered heterocyclic ring, or a 5 or 6 membered heteroaryl ring;

wherein the (1-6C)alkyl, (1-6C)alkoxy, (1-6C)alkoxy(1-6C)alkoxy and (1-6C)alkoxy(1-6C)alkoxy(1-6C)alkoxy groups and the (1-6C)alkyl groups of the mono(1-6C)alkylamino, di-[(1-6C)alkyl]amino, mono(1-6C)alkylcarbamoyl, di-[(1-6C)alkyl]carbamoyl and/or —N(R^{d})C(O)(1-6C)alkyl groups are optionally substituted by one or more hydroxy groups;

wherein the phenyl is optionally substituted by one or more groups independently selected from halo, (1-6C)alkyl, (1-6C)alkoxy, amino, mono(1-6C)alkylamino or di-[(1-6C)alkyl]amino,di(1-6C)alkylamino, wherein a (1-6C)alkyl and the (1-6C)alkoxy groups are optionally substituted by one or more groups independently selected from hydroxy, amino, mono(1-6C)alkylamino or di-[(1-6C)alkyl]amino;di-(1-6C)alkylamino;

and wherein any heterocyclic and heteroaryl rings within R¹ and/or R² are optionally independently substituted by one or more of the following: (1-4C)alkyl, (1-4C)alkoxy, (1-4C)alkoxy, (1-4C)alkoxy(1-4C)alkyl, hydroxy, amino, mono(1-6C)alkylamino or di-[(1-6C)alkyl]amino, mono(1-6C)alkylamino, di-[(1-6C)alkyl]amino, a saturated or partially saturated 3 to 7 membered heterocyclic ring, or —C(O)(CH₂)_{z}Y wherein z is 0, 1, 2 or 3 and Y is selected from hydrogen, hydroxy, (1-4C)alkoxy, (1-4C)alkoxy, amino, mono(1-6C)alkylamino, di-[(1-6C)alkyl]amino or a saturated or partially saturated 3 to 7 membered heterocyclic ring;

and provided that when R¹ and/or R² is a (1C)alkanoyl group, then the (1C)alkanoyl is not substituted by fluoro or hydroxy;
R³ and R⁴ are independently selected from hydrogen, (1-6C)alkyl or (1-6C)alkoxy, (1-6C)alkoxy wherein the (1-6C)alkyl and the (1-6C)alkoxy groups are optionally substituted by one or more groups independently selected from: fluoro, hydroxy, (1-6C)alkyl, (1-6C)alkoxy, amino, mono(1-6C)alkylamino, di-[(1-6C)alkyl]amino, mono(1-6C)alkylamino, di(1-6C)alkylamino, carbamoyl, mono(1-6C)alkylcarbamoyl or di-[(1-6C)alkyl]carbamoyl, a saturated or partially saturated 3 to 7 membered heterocyclic ring or a 5 or 6 membered heteroaryl ring, wherein said heterocyclic and heteroaryl rings are optionally independently substituted by one or more of the following: (1-4C)alkyl, (1-4C)alkoxy, hydroxy, amino, mono(1-6C)alkylamino or di-[(1-6C)alkyl]aminodi(1-6C)alkylamino or a saturated or partially saturated 3 to 7 membered heterocyclic ring; or one of R³ and R⁴ is as defined above and the other represents a group —NR¹R² as defined above;
A represents an aryl group or a 5 or 6 membered heteroaryl ring selected from furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl or 1,3,5-triazinyl;
R⁵ is selected from cyclopropyl, cyano, halo, (1-6C)alkoxy or (1-6C)alkyl, wherein the (1-6C)alkyl and the (1-6C)alkoxy groups are optionally substituted by cyano or by one or more fluoro;
n is 0, 1, 2 or 3;
L is attached meta or para on ring A with respect to the point of attachment of the ethynyl group and represents —N(R⁸)C(O)—(CR^{a}R^{b})ₓ-Z-(CR^{a}R^{b})_{y}—, —C(O)N(R⁹)—(CR^{a}R^{b})ₓ-Z-(CR^{a}R^{b})_{y}—, —C(R^{a})(R^{b})N(R⁸)C(O)—(CR^{a}R^{b})ₓ-Z-(CR^{a}R^{b})_{y}— or —C(R^{a})(R^{b})C(O)N(R⁹)—(CR^{a}R^{b})ₓ-Z-(CR^{a}R^{b})_{y}— wherein Z is a —O— or —N(R⁸)—
wherein x and y are independently 0, 1, 2 or 3, with the proviso that x+y <4, wherein R⁸ and R⁹ independently represent hydrogen or (1-6C)alkyl, wherein R^{a} and R^{b} independently represent hydrogen or (1-6C)alkyl or R^{a} and R^{b} together with the carbon atom to which they are attached represent (3-6C)cycloalkyl; and wherein a (1-6C)alkyl group in R^{a} and R^{b} is optionally substituted by halo, cyano, hydroxy or a saturated or partially saturated 3 to 7 membered heterocyclic ring;
B represents a (3-7C)cycloalkyl ring, a saturated or partially saturated 3 to 7 membered heterocyclic ring, an aryl group,or a 5 or 6 membered heteroaryl ring,ring; or a 8, 9 or 10 membered bicyclic group which optionally contains 1, 2, 3 or 4 heteroatoms independently selected from N, O and S and which is saturated, partially saturated or aromatic;
and when B is a (3-7C)cycloalkyl ring orring, a saturated or partially saturated 3 to 7 membered heterocyclic ring or a saturated or partially saturated 8, 9 or 10 membered bicyclic group, the rings and the bicyclic group optionally bear 1 or 2 oxo or thioxo substituents;
R⁶ is selected from halo, cyano, oxo, a (3-7C)cycloalkyl ring, a saturated or partially saturated 3 to 7 membered heterocyclic ring, —S(O)p-(1-6C)alkyl wherein p is 0, 1 or 2, —N(R^{c})C(O)(1-6C)alkyl ring and —N(R^{c})C(O)(1-6C)alkyl in which R^{c} is hydrogen or (1-6C)alkyl; or
R⁶ is selected from (1-6C)alkyl or (1-6C)alkoxy, (1-6C)alkyl, —S(O)ₚ-(1-6C)alkyl wherein
p is 0, 1 or 2, or (1-6C)alkoxy,
wherein the (1-6C)alkyl, —S(O)ₚ-(1-6C)alkyl and the (1-6C)alkoxy(1-6C)alkoxy groups are optionally substituted by one or more groups independently selected from: cyano, fluoro, hydroxy, (1-6C)alkoxy, (1-6C)alkoxy, amino, mono(1-6C)alkylamino, di-[(1-6C)alkyl]amino, di(1-6C)alkylamino, a (3-7C)cycloalkyl ring or a saturated or partially saturated 3 to 7 membered heterocyclic ring; and
wherein the (3-7C)cycloalkyl ring and saturated or partially saturated 3 to 7 membered heterocyclic ring are optionally independently substituted by one or more groups selected from (1-6C)alkyl or hydroxy(1-6C)alkyl; and
m is 0, 1, 2 or 3;
and salts or solvates thereof.
with the proviso that:
   (i) L cannot be —C(R^{a})(R^{b})C(O)N(R⁹)—, —N(R⁸)C(O)C(R^{a})(R^{b})—, —N(R⁸)C(O)O— or —N(R⁸)C(O)N(R⁸)—.

In particular, L is selected from N(R⁸)C(O)—, N(R⁸)C(O)—(CR^{a}R^{b})₂, —C(O)N(R⁹), —C(O)N(R⁹)—CR^{a}R^{b}), —C(R^{a})(R^{b})—N(R⁸)C(O)— or —C(R^{a})(R^{b})—C(O)N(R⁹)—(CR^{a}R^{b})—.

Suitably, R³ and R⁴ are other than a group NR¹R².

Suitably R^{y} is a group NR¹R² where R¹ and R² are as defined above.

Thus, in a particular aspect, the invention provides a compound of formula (IA):

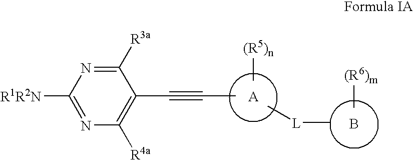

wherein:
R¹, R², R⁵, A, L, B, m and n are as defined above,
R^{3a} and R^{4a} are groups R³ and R⁴ as defined above, and
R⁶ is selected from halo, cyano, oxo, a (3-7C)cycloalkyl ring, a saturated or partially saturated 3 to 7 membered heterocyclic ring and —N(R^{c})C(O)(1-6C)alkyl in which R^{c} is hydrogen or (1-6C)alkyl;
or R⁶ is selected from (1-6C)alkyl, —S(O)ₚ-(1-6C)alkyl wherein p is 0, 1 or 2, or (1-6C)alkoxy,
wherein the (1-6C)alkyl, —S(O)ₚ-(1-6C)alkyl and the (1-6C)alkoxy groups are optionally substituted by one or more groups independently selected from: cyano, fluoro, hydroxy, (1-6C)alkoxy, amino, mono(1-6C)alkylamino, di(1-6C)alkylamino, a (3-7C)cycloalkyl ring or a saturated or partially saturated 3 to 7 membered heterocyclic ring; and
wherein the (3-7C)cycloalkyl ring and saturated or partially saturated 3 to 7 membered heterocyclic ring are optionally independently substituted by one or more groups selected from (1-6C)alkyl;
and salts or solvates thereof.

In a particular embodiment, n there is provided a compound of the Formula IA as defined above wherein:

R¹, R², A, B, L, R⁵, R⁶, m and n are as defined above in relation to formula (IA) and R^{3a} and R^{4a} are independently selected from hydrogen, (1-6C)alkyl or (1-6C)alkoxy, wherein the (1-6C)alkyl and the (1-6C)alkoxy groups are optionally substituted by one or more groups independently selected from fluoro, hydroxy, (1-6C)alkyl, (1-6C)alkoxy, amino, mono(1-6C)alkylamino, di-[(1-6C)alkyl]amino, carbamoyl, mono(1-6C)alkylcarbamoyl, di-[(1-6C)alkyl]carbamoyl, a saturated or partially saturated 3 to 7 membered heterocyclic ring or a 5 or 6 membered heteroaryl ring, wherein said heterocyclic and heteroaryl rings are optionally independently substituted by one or more of the following: (1-4C)alkyl, (1-4C)alkoxy, hydroxy, amino, mono(1-6C)alkylamino or di-[(1-6C)alkyl]amino or a saturated or partially saturated 3 to 7 membered heterocyclic ring.

In a further embodiment, R^{x} is a group NR¹R² where R¹ and R² are as defined above.

Thus according to a further aspect of the present invention there is provided a compound of the Formula IB:

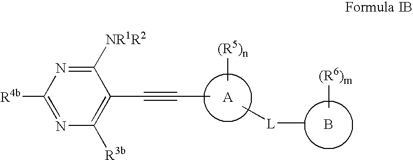

wherein:
R¹, R², R⁵, R⁶, A, L, B, m and n are as defined above in relation to formula (I)
R^{3b} are R^{4b} are independently selected from hydrogen, (1-6C)alkyl or (1-6C)alkoxy, wherein the (1-6C)alkyl and the (1-6C)alkoxy groups are optionally substituted by one or more groups independently selected from fluoro, hydroxy, (1-6C)alkyl, (1-6C)alkoxy, amino, mono(1-6C)alkylamino, di-[(1-6C)alkyl]amino, carbamoyl, mono(1-6C)alkylcarbamoyl or di-[(1-6C)alkyl]carbamoyl, a saturated or partially saturated 3 to 7 membered heterocyclic ring or a 5 or 6 membered heteroaryl ring, wherein said heterocyclic and heteroaryl rings are optionally independently substituted by one or more of the following: (1-4C)alkyl, (1-4C)alkoxy, hydroxy, amino, mono(1-6C)alkylamino or di-[(1-6C)alkyl]amino or a saturated or partially saturated 3 to 7 membered heterocyclic ring;
or one of R^{3b} and R^{4b} is as defined above and the other represents a group —NR¹R² as defined above.

In a particular embodiment, R^{3b} and R^{4b} are other than —NR¹R².

In another particular embodiment, the compounds of the Formula IB is a compound of the Formula IBi:

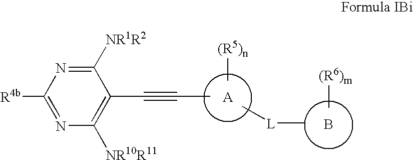

wherein:
R¹, R², R^{4b}, R⁵, R⁶, A, L, B, m and n are as defined above in relation to formula (IB) and R¹⁰ and R¹¹ are independently selected from hydrogen or (1-6C)alkyl;
and salts or solvates thereof, particularly pharmaceutically acceptable salts thereof with the proviso that:
L cannot be —C(R^{a})(R^{b})C(O)N(R⁹)—, —N(R⁸)C(O)C(R^{a})(R^{b})—, —N(R⁸)C(O)O— or —N(R⁸)C(O)N(R⁸)—.

In this specification the generic term “alkyl” includes both straight-chain and branched-chain alkyl groups such as propyl, isopropyl and tert-butyl. However references to individual alkyl groups such as “propyl” are specific for the straight-chain version only, references to individual branched-chain alkyl groups such as “isopropyl” are specific for the branched-chain version only. An analogous convention applies to other generic terms, for example (1-6C)alkoxy includes methoxy, ethoxy and isopropoxy, (1-6C)alkylamino includes methylamino, isopropylamino and ethylamino, and di-[(1-6C)alkyl]amino includes dimethylamino, diethylamino and N-methyl-N-isoproylamino. The generic term aryl refers to phenyl or naphthyl, particularly phenyl.

It is to be understood that, insofar as certain of the compounds of Formula I defined above may exist in optically active or racemic forms by virtue of one or more asymmetric carbon atoms, the invention includes in its definition any such optically active or racemic form which possesses the above-mentioned activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter. Suitable values for the generic radicals referred to above include those set out below.

Suitable 5 or 6 membered heteroaryl rings include, for example furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, 1,4,5-triazinyl or pyrazinyl. Particular 5 or 6 membered heteroaryl rings include imidazolyl, pyridyl, thiazolyl, thiadiazolyl, pyrimidinyl, isoxazolyl, isothiazolyl and pyrazolyl. Suitable saturated or partially saturated 3 to 7 membered heterocyclic rings include, for example oxiranyl, oxetanyl, tetrahydrofuranyl, tetrahydropyranyl, 2,3-dihydro-1,3-thiazolyl, 1,3-thiazolidinyl, 1,3-oxazolidinyl, oxepanyl, pyrrolinyl, pyrrolidinyl, morpholinyl, thiamorpholinyl (perhydro-1,4-thiazinyl), (8-oxa-3-azabicyclo[3.2.1]octyl), (7-oxa-3-azabicyclo[3.1.1]heptyl), perhydroazepinyl,perhydrooxazepinyl, tetrahydro-1,4-thiazinyl, 1-oxotetrahydrothienyl, 1,1-dioxotetrahydro-1,4-thiazinyl, piperidinyl, homopiperidinyl, piperazinyl, homopiperazinyl, dihydropyridinyl, tetrahydropyridinyl, dihydropyrimidinyl or tetrahydropyrimidinyl, preferably tetrahydrofuranyl, tetrahydropyranyl, pyrrolidinyl, morpholinyl, 1,1-dioxotetrahydro-4H-1,4-thiazinyl, piperidinyl or piperazinyl, more preferably tetrahydrofuran-3-yl, tetrahydropyran-4-yl, pyrrolidin-3-yl, morpholino, 1,1-dioxotetrahydro-4H-1,4-thiazin-4-yl, piperidino, piperidin-4-yl or piperazin-1-yl. A suitable value for such a group which bears 1 or 2 oxo or thioxo substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. The saturated or partially saturated 3 to 7 membered heterocyclic rings are optionally substituted by one or more (C1-6) alkyl groups and/or by one or more hydroxy. For avoidance of doubt it will be understood that this definition includes tautomers of hydroxy substituted ring systems where the hydroxy tautomerizes to an oxo group.

Suitable 8, 9 or 10 membered bicyclic groups include thieno[2,3-b]furanyl, imidazolo[2,1-b]thiazolyl, dihydrocyclopentathiazolyl, tetrahydrocyclopenta[c]pyrazolyl, furo[3,2-b]furanyl, pyrrolopyrrole, thienopyrazolyl, thieno[2,3-b]thiophenyl, indolizinyl, indolyl, isoindolyl, 3H-indolyl, indolin-yl, benzo[b]furanyl, benzo[b]thiophenyl, 1H-indazolyl, benzimidazolyl, benzthiazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl, chromanyl, isochromanyl, indenyl, naphthalenyl, 2,3-dihydro-1,4-benzodioxinyl, 1,3-benzodioxol-5-yl, decalin and norbornane. Particular 8, 9 or 10 membered bicyclic groups include thieno[2,3-b]furanyl, indolizinyl, indolyl, isoindolyl, 3H-indolyl, indolin-yl, benzo[b]furanyl, benzo[b]thiophenyl, 1H-indazolyl, benzimidazolyl, benzthiazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl, chromanyl, isochromanyl, indenyl, naphthalenyl, 2,3-dihydro-1,4-benzodioxinyl and 1,3-benzodioxol-5-yl.

The bicyclic groups are optionally substituted by one or more groups R⁶ as hereinbefore defined.

The group A may particularly be attached to the ethynyl group via a carbon atom in the aryl group or in the 5 or 6 membered heteroaryl ring. The group B may particularly be attached to the group L via a carbon atom.

Suitable values for any of the the substituents herein, for example the ‘R’ groups (R¹ to R⁶) or for various groups within a A, B or L group include

| | |
|---|---|
| for halo | fluoro, chloro, bromo and iodo; |
| for (1-6C)alkyl: | methyl, ethyl, propyl, isopropyl and tert-butyl; |
| for (1-6C)alkoxy: | methoxy, ethoxy, propoxy, isopropoxy and |
| | butoxy; |
| for (1-6C)alkylsulfonyl: | methylsulfonyl and ethylsulfonyl; |
| for (1-6C)alkylamino: | methylamino, ethylamino, propylamino, |
| | isopropylamino and butylamino; |
| for di-[(1-6C)alkyl]amino: | dimethylamino, diethylamino, N-ethyl- |
| | N-methylamino and diisopropylamino; |
| for (1-6C)alkoxycarbonyl: | methoxycarbonyl, ethoxycarbonyl, |
| | propoxycarbonyl and tert-butoxycarbonyl; |
| for (1-6C)alkanoyl: | formyl, acetyl and propionyl; |
| for (1-6C)alkoxycarbonyl | methoxycarbonyl, propoxycarbonyl, |
| | isopropoxycarbonyl, t-butoxycarbonyl; |
| for hydroxy(1-6C)alkyl: | hydroxymethyl, 2-hydroxyethyl, 1-hydroxyethyl |
| | and 3-hydroxypropyl; |
| for (1-6C)alkoxy(1-6C)alkyl: | methoxymethyl, ethoxymethyl, 1-methoxyethyl, |
| | 2-methoxyethyl, 2-ethoxyethyl and 3- |
| | methoxypropyl; |
| for (3-7C)cycloalkyl: | cyclopropyl, cyclobutyl, cyclopentyl, cyclohexyl |
| | and cycloheptyl; |
| for (1-6C)alkoxy(1-6C)alkoxy: | methoxymethoxy, methoxyethoxy, |
| | methoxypropoxy, methoxybutoxy, |
| | methoxyhexoxy, ethoxyethoxy, ethoxypropoxy, |
| | ethoxybutoxy, propoxypropoxy and |
| | propoxybutoxy; |
| for (1-6C)alkoxy(1-6C)alkoxy(1-6C)alkoxy: | methoxymethoxymethoxy, |
| | methoxyethoxyethoxy, methoxypropoxymethoxy, |
| | methoxybutoxyethoxy, methoxyhexoxymethoxy, |
| | ethoxyethoxyethoxy, ethoxypropoxyethoxy, |
| | ethoxybutoxymethoxy, propoxypropoxymethoxy |
| | and propoxybutoxymethoxy; |
| for mono(1-6C)alkylcarbamoyl: | N-methylcarbamoyl, N-ethylcarbamoyl and |
| | N-propylcarbamoyl; and |
| for di-[(1-6C)alkyl]carbamoyl: | N,N-dimethylcarbamoyl, N-ethyl- |
| | N-methylcarbamoyl and N,N-diethylcarbamoyl. |

When in this specification reference is made to a (1-4C)alkyl group it is to be understood that such groups refer to alkyl groups containing up to 4 carbon atoms. A skilled person will realise that representative examples of such groups are those listed above under (1-4C)alkyl that contain up to 4 carbon atoms, such as methyl, ethyl, propyl, isopropyl, butyl and tert-butyl. Similarly, reference to a (1-3C)alkyl group refers to alkyl groups containing up to 3 carbon atoms such as methyl, ethyl, propyl and isopropyl. A similar convention is adopted for the other groups listed above such as (1-4C)alkoxy, (2-4C)alkenyl, (2-4C)alkynyl and (1-4C)alkanoyl.

It is to be understood that certain compounds of the formula I may exist in solvated as well as unsolvated forms such as, for example, hydrated forms. It is to be understood that the invention encompasses all such solvated forms which exhibit an inhibitory effect on a Tie2 receptor tyrosine kinase.

It is also to be understood that certain compounds of the formula I may exhibit polymorphism, and that the invention encompasses all such forms which exhibit an inhibitory effect on a Tie2 receptor tyrosine kinase.

It is also to be understood that the invention relates to all tautomeric forms of the compounds of the formula I forms which exhibit an inhibitory effect on a Tie2 receptor tyrosine kinase.

Whilst pharmaceutically-acceptable salts of compounds of the invention are preferred, other non-pharmaceutically-acceptable salts of compounds of the invention may also be useful, for example in the preparation of pharmaceutically-acceptable salts of compounds of the invention.

A suitable pharmaceutically acceptable salt of a compound of the formula I is, for example, an acid-addition salt of a compound of the formula I, for example an acid-addition salt with an inorganic or organic acid such as hydrochloric, hydrobromic, sulfuric, trifluoroacetic, citric or maleic acid; or, for example, a salt of a compound of the formula I which is sufficiently acidic, for example an alkali or alkaline earth metal salt such as a calcium or magnesium salt, or an ammonium salt, or a salt with an organic base such as methylamine, dimethylamine, trimethylamine, piperidine, morpholine or tris-(2-hydroxyethyl)amine.

Also provided as a further aspect of the invention are pro-drugs of compounds of the invention as herein before or herein after defined. Compounds of the invention may be administered in the form of a pro-drug which is broken down in the human or animal body to give a compound of the Formula (I). Examples of pro-drugs include in-vivo hydrolysable esters of a compound of the Formula (I).

Various forms of pro-drugs are known in the art. For examples of such pro-drug derivatives, see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs” , by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

An in-vivo hydrolysable ester of a compound of the Formula (I) containing a hydroxy group is, for example, a pharmaceutically-acceptable ester which is hydrolysed in the human or animal body to produce the parent acid or alcohol. Suitable pharmaceutically-acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters, for example 5-methyl-1,3-dioxolen-2-onylmethyl; and C₁₋₆alkoxycarbonyloxyethyl esters.

An in-vivo hydrolysable ester of a compound of the Formula (I) containing a hydroxy group includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxy-methoxy. A selection of in-vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-allylcarbamoyl (to give carbamates), dialkylaminoacetyl and carboxyacetyl.

Particular novel compounds of the invention include, for example, compounds of the formula I, or salts, particularly pharmaceutically acceptable salts thereof, wherein, unless otherwise stated, each of R¹, R², R³, R⁴, R⁵, R⁶, A, B, L, m and n has any of the meanings defined hereinbefore or in paragraphs (a) to (lllll) hereinafter:-
(a) L is attached meta on ring A with respect to the point of attachment of the ethynyl group;
(a′) L is attached para on ring A with respect to the point of attachment of the ethynyl group;
(b) L is —N(R⁸)C(O)—(CR^{a}R^{b})ₓ-Z-(CR^{a}R^{b})_{y}— wherein x and y are as defined above, and Z is —O— or —N(H)—, and R^{a}, R^{b} and R⁸ are independently selected from hydrogen and (1-6C)alkyl (particularly R^{a}, R^{b} and R⁸ are all hydrogen);
(b′) L is —C(O)N(R⁹)—(CR^{a}R^{b})ₓ-Z-(CR^{a}R^{b})_{y}— wherein x and y are as defined above, and Z is —O— or —N(H)—, and R^{a}, R^{b} and R⁹ are independently selected from hydrogen and (1-6C)alkyl (particularly R^{a}, R^{b} and R⁹ are all hydrogen);
(b″) L is —C(R^{a})(R^{b})—N(R⁸)C(O)—(CR^{a}R^{b})ₓ-Z-(CR^{a}R^{b})_{y}— wherein x and y are as defined above, and Z is —O— or —N(H)—, and R^{a}, R^{b} and R⁸ are independently selected from hydrogen and (1-6C)alkyl (particularly R^{a}, R^{b} and R⁸ are all hydrogen);
(b′″) L is —C(R^{a})(R^{b})—C(O)N(R⁹)—(CR^{a}R^{b})ₓ-Z-(CR^{a}R^{b})_{y}— wherein x and y are as defined above, and Z is —O— or —N(H)—, and R^{a}, R^{b} and R⁹ are independently selected from hydrogen and (1-6C)alkyl (particularly R^{a}, R^{b} and R⁹ are all hydrogen);
(c) L is —N(R⁸)C(O)—(CR^{a}R^{b})ₓ—O—(CR^{a}R^{b})_{y}— wherein x and y are as defined above, and R^{a}, R^{b} and R⁸ are independently selected from hydrogen and (1-6C)alkyl (particularly R^{a}, R^{b} and R⁸ are all hydrogen);
(c′) L is —C(O)N(R⁹)—(CR^{a}R^{b})ₓ—O—(CR^{a}R^{b})_{y}— wherein x and y are as defined above, and R^{a}, R^{b} and R⁹ are independently selected from hydrogen and (1-6C)alkyl (particularly R^{a}, R^{b} and R⁹ are all hydrogen);
(c″) L is —C(R^{a})(R^{b})—N(R⁸)C(O)—(CR^{a}R^{b})ₓ—O—(CR^{a}R^{b})_{y}— wherein x and y are as defined above, and R^{a}, R^{b} and R⁸are independently selected from hydrogen and (1-6C)alkyl (particularly R^{a}, R^{b} and R⁸are all hydrogen);
(c′″) L is —C(R^{a})(R^{b})—C(O)N(R⁹)—(CR^{a}R^{b})ₓ—O—(CR^{a}R^{b})_{y}— wherein x and y are as defined above, and R^{a}, R^{b} and R⁹ are independently selected from hydrogen and (1-6C)alkyl (particularly R^{a}, R^{b} and R⁹ are all hydrogen);
(d) L is —N(R⁸)C(O)—(CR^{a}R^{b})ₓ—N(R⁸)—(CR^{a}R^{b})_{y}— wherein R^{a}, R^{b} and R⁸ are independently selected from hydrogen and (1-6C)alkyl (particularly R^{a}, R^{b} and R⁸ are all hydrogen);
(d′) L is —C(O)N(R⁹)—(CR^{a}R^{b})ₓ—N(R⁸)—(CR^{a}R^{b})_{y}— wherein R^{a}, R^{b}, R⁸and R⁹ are independently selected from hydrogen and (1-6C)alkyl (particularly R^{a}, R^{b} and R⁹ are all hydrogen);
(d″) L is —C(R^{a})(R^{b})—N(R⁸)C(O)—(CR^{a}R^{b})ₓ—N(R⁸)—(CR^{a}R^{b})_{y}— wherein R^{a}, R^{b} and R⁸ are independently selected from hydrogen and (1-6C)alkyl (particularly R^{a}, R^{b} and R⁸ are all hydrogen);
(d′″) L is —C(R^{a})(R^{b})—C(O)N(R⁹)—(CR^{a}R^{b})ₓ—N(R⁸)—(CR^{a}R^{b})_{y}— wherein R^{a}, R^{b}, R⁸ and R⁹ are independently selected from hydrogen and (1-6C)alkyl (particularly R^{a}, R^{b}, R⁸ and R⁹ are all hydrogen);
(e) L is —N(R⁸)C(O)—(CR^{a}R^{b})ₓ—O— wherein x is as defined above, and R^{a}, R^{b} and R⁸ are independently selected from hydrogen and (1-6C)alkyl (particularly R^{a}, R^{b} and R⁸ are all hydrogen);
(e′) L is —C(O)N(R⁹)—(CR^{a}R^{b})ₓ—O— wherein x is as defined above, and R^{a}, R^{b} and R⁹ are independently selected from hydrogen and (1-6C)alkyl (particularly R^{a}, R^{b} and R⁹ are all hydrogen);

(e′″) L is —C(R^{a})(R^{b})—N(R)C(O)—(CR^{a}R^{b})ₓ—O— wherein x is as defined above, and R^{a}, R^{b} and R⁸ are independently selected from hydrogen and (1-6C)alkyl (particularly R^{a}, R^{b} and R⁸ are all hydrogen);

(e″″) L is —C(R^{a})(R^{b})—C(O)N(R⁹)—(CR^{a}R^{b})ₓ—O— wherein x is as defined above, and R^{a}, R^{b} and R⁹ are independently selected from hydrogen and (1-6C)alkyl (particularly R^{a}, R^{b} and R⁹ are all hydrogen);
(f) L is —N(R⁸)C(O)—(CR^{a}R^{b})ₓ—N(H)— wherein x is as defined above, and R^{a}, R^{b} and R⁸ are independently selected from hydrogen and (1-6C)alkyl (particularly R^{a}, R^{b} and R⁸ are all hydrogen);
(f′) L is —C(O)N(R⁹)—(CR^{a}R^{b})ₓ—N(H)— wherein x is as defined above, and R^{a}, R^{b} and R⁹ are independently selected from hydrogen and (1-6C)alkyl (particularly R^{a}, R^{b} and R⁹ are all hydrogen);
(f″) L is —C(R^{a})(R^{b})—N(R⁸)C(O)—(CR^{a}R^{b})ₓ—N(H)— wherein x is as defined above, and R^{a}, R^{b} and R⁸ are independently selected from hydrogen and (1-6C)alkyl (particularly R^{a}, R^{b}, R⁸ and R⁹ are all hydrogen);
(f′″) L is —C(R^{a})(R^{b})—C(O)N(R⁹)—(CR^{a}R^{b})ₓ—N(H)— wherein x is as defined above, and R^{a}, R^{b} and R⁹ are independently selected from hydrogen and (1-6C)alkyl (particularly R^{a}, R^{b} and R⁹ are all hydrogen);
(g) L is —N(R⁸)C(O)—(CR^{a}R^{b})ₓ— wherein x is as defined above (particularly x is 1 or 2) and wherein R^{a}, R^{b} and R⁸ are independently selected from hydrogen and (1-6C)alkyl (particularly R^{a}, R^{b} and R⁸ are all hydrogen);
(g′) L is —C(O)N(R⁹)—(CR^{a}R^{b})ₓ— wherein x is as defined above (particularly x is 1 or 2) and wherein R^{a}, R^{b} and R⁹ are independently selected from hydrogen and (1-6C)alkyl (particularly R^{a}, R^{b} and R⁹ are all hydrogen);
(g″) L is —C(R^{a})(R^{b})—N(R⁸)C(O)—(CR^{a}R^{b})ₓ—, wherein x is as defined above (particularly x is 1 or 2) and wherein R^{a}, R^{b} and R⁸ are independently selected from hydrogen and (1-6C)alkyl (particularly R^{a}, R^{b} and R⁸ are all hydrogen);
(g′″) L is —C(R^{a})(R^{b})—C(O)N(R⁹)—(CR^{a}R^{b})ₓ—, particularly —CH₂—C(O)N(R⁹)— wherein x is as defined above (particularly x is 1 or 2) and wherein R^{a}, R^{b} and R⁹ are independently selected from hydrogen and (1-6C)alkyl (particularly R^{a}, R^{b} and R⁹ are all hydrogen);
(g″″) L is —C(O)—N(H)—.
(g″″) L is —C(O)—N(H)—, —N(H)—C(O)—, —C(O)—N(H)—CH₂—, —CH₂—N(H)—C(O)—, —N(H)—C(O)—(CH₂)₂ or —CH₂—C(O)—N(H)—CH₂—,
(g′″″) L is —N(H)C(O)—.
(g″″″) L is —C(O)N(H)CH₂—.
(g′″″″) L is —CH₂N(H)C(O)—.
(g″″″″) L is —N(H)C(O)CH₂CH₂—.
(g′″″″″) L is CH₂—C(O)—N(H)—CH₂—,
(h) R^{a} and R^{b} each represent hydrogen;
(h′) R^{a} and R^{b} independently represent hydrogen or (1-6C)alkyl, wherein a (1-6C)alkyl group in R^{a} and R^{b} is optionally substituted by hydroxy or a saturated or partially saturated 3 to 7 membered heterocyclic ring;
(h″) R^{a} and R^{b} independently represent hydrogen or (1-6C)alkyl, wherein a (1-6C)alkyl group in R^{a} and R^{b} is optionally substituted by hydroxy or a saturated or partially saturated 5 to 6 membered heterocyclic ring;
(h′″) R^{a} and R^{b} independently represent hydrogen, methyl or ethyl, wherein a (1-6C)alkyl group in R^{a} and R^{b} is optionally substituted by hydroxy or pyrrolin-1-yl;
(i) A is selected from phenyl, furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl and 1,3,5-triazinyl;
(i′) A is selected from phenyl, thiazolyl, thiadiazolyl, pyridyl and pyrimidinyl;
(j) A is phenyl or pyridyl
(k) A is phenyl or pyridyl, wherein the nitrogen in the pyridyl ring is in the 3-position relative to the alkyne bond.
(l) A is thienyl or thiazolyl;
(l′) A is phenyl, pyridyl, thienyl or thiazolyl;
(l″) A is phenyl;
(m) A is pyridyl;
(m′) A is thiazolyl;
(n) A is phenyl or pyridyl and n is 0;
(n′) A is phenyl or thiazolyl and n is 0;
(o) A is phenyl and n is 0 or n is 1 and R⁵ is (1-4C)alkyl;
(p) A is pyridyl and n is 0 or n is 1 and R⁵ is (1-4C)alkyl;
(p′) A is thiazolyl and n is 0 or n is 1 and R⁵ is (1-4C)alkyl;
(p″) A is thienyl and n is 0 or n is 1 and R⁵ is (1-4C)alkyl;
(q) A is selected from phenyl, oxazolyl, imidazolyl, pyrrolyl, pyrazolyl, thienyl, thiazolyl, isothiazolyl, thiadiazolyl, pyridyl, pyridazinyl, pyrazinyl and pyrimidyl.
(r) When B is a (3-7C)cycloalkyl ring then B is selected from cyclopropyl, cyclobutyl, cyclopentyl and cyclohexyl;
(s) When B is a saturated or partially saturated 3 to 7 membered heterocyclic ring then B is selected from oxetanyl, azetidinyl, thietanyl, pyrrolidinyl, morpholinyl, 1,3-dioxolanyl, tetrahydrofuranyl, piperidyl, piperazinyl, thiomorpholinyl, tetrahydropyranyl, homopiperazinyl, pyrrolinyl, imidazolinyl, pyrazolinyl, pyranyl, tetrahydropyridinyl, 1,2,4-oxadiazolyl and dihydrothiopyranyl;
(t) When B is a 5 or 6 membered heteroaryl ring then B is selected from furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl or 1,3,5-triazinyl;
(u) When B is an 8, 9 or 10 membered bicyclic group which optionally contains 1,2,3 or 4 heteroatoms independently selected from N, O and S and which is saturated, partially saturated or aromatic then B is selected from 2,3-dihydro-1H-indenyl, benzodioxinyl, 1,2,3,4-tetrahydronaphthalenyl, 1,2,3,4-tetrahydropentalene, benzofuranyl, 2,3-dihydrobenzofuranyl, benzimidazolyl, benzthienyl, benzthiazolyl, benzisothiazolyl, benzoxazolyl, benzisoxazolyl, pyridoimidazolyl, pyrimidoimidazolyl, quinolinyl, isoquinolinyl, quinoxalinyl, quinazolinyl, phthalazinyl, cinnolinyl, indolyl, and naphthyridinyl.
or B is a group of the formula:

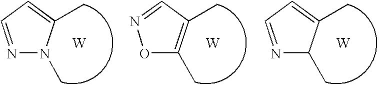

wherein W is a 5-7 membered ring (including the bridging atoms), said W ring comprising carbon atoms or optionally further heteroatoms independently selected from oxygen, nitrogen and sulphur, wherein said bicyclic ring contains no more that 4 heteroatoms in total. Examples of such rings include: pyrazolo[1,5-a]pyridinyl, pyrazolo[1,5-c]pyrimidinyl, pyrazolo[1,5-a][1,3,5]triazinyl, 4,5-dihydropyrazolo[1,5-a]pyridinyl, 4H-pyrazolo[5,1-c][1,4]thiazinyl, 4H-pyrazolo[5,1-c][1,4]oxazinyl, 1,2-benzisoxazolyl, isoxazolo[5,4-b]pyridinyl, isoxazolo[5,4-d]pyrimidinyl, 4H-thiopyrano[3,4-d]isoxazolyl, 4H-pyrano[3,4-d]isoxazolyl, 7aH-indolyl, 7aH-pyrrolo[2,3-b]pyridinyl, 7aH-pyrrolo[2,3-b]pyrimidinyl, 4,7a-dihydrothiopyrano[4,3-b]pyrrolyl and 4,7a-dihydropyrano[4,3-b]pyrrolyl.
(v) B is aryl, particularly phenyl;
(w) B is a saturated or partially saturated 3 to 7 (particularly 4 to 6) membered heterocyclic ring that contains one or two heteroatoms (particularly one heteroatom) selected from oxygen and nitrogen;
(x) B is a a 5 or 6 membered heteroaryl ring;
(y) B is a 8, 9 or 10 membered bicyclic group which optionally contains 1, 2 or 3 (particularly 1 or 2) heteroatoms independently selected from N and O and which is saturated, partially saturated or aromatic;
(z) B is selected from a saturated or partially saturated 4 to 6 membered heterocyclic ring, an aryl group, a 5 or 6 membered heteroaryl ring selected from furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl or 1,3,5-triazinyl, or a 8, 9 or 10 membered bicyclic group which optionally contains 1, 2, 3 or 4 heteroatoms independently selected from N, O and S and which is saturated, partially saturated or aromatic;
(aa) B is selected from a saturated or partially saturated 4 to 6 membered heterocyclic ring, an aryl group or a 5 or 6 membered heteroaryl ring selected from furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl or 1,3,5-triazinyl;
(bb) B is selected from a saturated or partially saturated 4 to 6 membered heterocyclic ring, or a 5 or 6 membered heteroaryl ring selected from furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl or 1,3,5-triazinyl;
(cc) B is selected from cyclopropyl, cyclobutyl, cyclopentyl, cyclohexyl, phenyl, piperidinyl, tetrahydrofuranyl, tetrahydropyranyl, 1,4-dioxanyl, morpholinyl, pyrrolidinyl, piperidinyl, furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazinyl, 2,3-dihydro-1,4-benzodioxinyl and 1,3-benzodioxol-5-yl;
(dd) B is selected from phenyl, tetrahydopyranyl, tetrahydrofuranyl, morpholinyl, thiomorpholinyl, furyl, pyrrolidinyl, pyridyl and pyrimidinyl;
(dd′) B is selected from phenyl, cyclobutyl, tetrahydopyranyl, tetrahydrofuranyl, 1,4-dioxanyl, morpholinyl, furyl, pyrrolidinyl, piperidinyl, pyrazolyl, isothiazolyl, isoxazolyl, pyridyl, pyrimidinyl, pyrazinyl and pyridazinyl;
(dd″) B is selected from cyclohexyl, phenyl, tetrahydopyranyl, tetrahydrofuranyl, morpholinyl, thiomorpholinyl, furyl, pyrrolidinyl, pyridyl and pyrimidinyl;
(dd′″) B is selected from phenyl, imidazolyl, thienyl, and isoxazolyl;
(dd″″) B is selected from phenyl, isoxazolyl or tetrahydopyranyl.
(dd″″″) B is selected from isoxazolyl, pyrazolyl, tetrahydopyranyl, phenyl and benzodioxolyl
(ee) B is selected from phenyl, cyclobutyl, 2,3-di-hydro-indenyl, tetrahydopyranyl, tetrahydrofuranyl, piperidinyl, 1,4-dioxanyl, morpholinyl, thiomorpholinyl, pyrrolidinyl, piperidinyl, furyl, imidazolyl, thienyl, pyrazolyl, isothiazolyl, thiadiazolyl, isoxazolyl, pyridyl, pyrimidinyl, pyrazinyl, pyridazinyl, benzodioxinyl, benzodioxolyl or tetrahydropyranyl.
(ee′) B is selected from phenyl, cyclohexyl, cyclobutyl, 2,3-di-hydro-indenyl, tetrahydopyranyl, tetrahydrofuranyl, piperidinyl, 1,4-dioxanyl, morpholinyl, thiomorpholinyl, pyrrolidinyl, piperidinyl, furyl, imidazolyl, thienyl, pyrazolyl, isothiazolyl, thiadiazolyl, isoxazolyl, pyridyl, pyrimidinyl, pyrazinyl, pyridazinyl, benzodioxinyl, benzodioxolyl or tetrahydropyranyl.
(ff) B is selected from piperidinyl, phenyl, isoxazolyl, isothiazolyl, thiadiazolyl, pyrazolyl and pyridyl;
(gg) B is selected from phenyl, pyrazolyl, thiadiazolyl and isoxazolyl;
(hh) Bis selected from isoxazolyl, thiadiazolyl and pyrazolyl;
(ii) Bis selected from isoxazolyl and pyrazolyl;
(jj) B is phenyl;
(kk) B is isoxazolyl;
(ll) B is pyrazolyl;
(mm) R¹ and R² are independently selected from hydrogen, phenyl(CH₂)ᵤ— wherein u is 0, 1, 2, 3, 4, 5 or 6, (1-6C)alkanoyl, (1-6C)alkyl, (1-6C)alkoxycarbonyl, (3-6C)cycloalkyl(CH₂)ᵥ— in which v is 0, 1, 2, 3, 4, 5 or 6, or a 5 or 6 membered heteroaryl ring;

wherein the (1-6C)alkyl, the (1-6C)alkanoyl and the (3-6C)cycloalkyl groups are optionally substituted by one or more groups (for example 1 or 2), which may be the same or different, selected from fluoro, hydroxy, (1-6C)alkyl, (1-6C)alkoxy, (1-6C)alkoxy(1-6C)alkoxy, (1-6C)alkoxy(1-6C)alkoxy(1-6C)alkoxy, amino, mono(1-6C)alkylamino, di-[(1-6C)alkyl]amino, carbamoyl, mono(1-6C)alkylcarbamoyl, di-[(1-6C)alkyl]carbamoyl or—N(R^{d})C(O)(1-6C)alkyl in which R^{d} is hydrogen or (1-6C)alkyl, or a saturated or partially saturated 3 to 7 membered heterocyclic ring, or a 5 or 6 membered heteroaryl ring, wherein the (1-6C)alkoxy, (1-6C)alkoxy(1-6C)alkoxy and (1-6C)alkoxy(1-6C)alkoxy(1-6C)alkoxy groups and the (1-6C)alkyl groups of the mono(1-6C)alkylamino, di-[(1-6C)alkyl]amino, mono(1-6C)alkylcarbamoyl, di-[(1-6C)alkyl]carbamoyl and/or —N(R^{d})C(O)(1-6C)alkyl groups are optionally substituted by one or more (for example 1 or 2) hydroxy groups;

wherein the phenyl is optionally substituted by one or more groups (for example 1 or 2), which may be the same or different, selected from halo, (1-6C)alkyl, (1-6C)alkoxy, amino, mono(1-6C)alkylamino or di-[(1-6C)alkyl]amino, wherein the (1-6C)alkyl or (1-6C)alkoxy are optionally substituted by one or more groups (for example 1 or 2), which may be the same or different, selected from hydroxy, amino, mono(1-6C)alkylamino or di-[(1-6C)alkyl]amino;

and wherein any heterocyclic and heteroaryl rings within R¹ and/or R² are optionally independently substituted by one or more groups (for example 1 or 2), which may be the same or different selected from (1-4C)alkyl, (1-4C)alkoxy, (1-4C)alkoxy(1-4C)alkyl, hydroxy, amino, mono(1-6C)alkylamino or di-[(1-6C)alkyl]amino, or a saturated or partially saturated 3 to 7 membered heterocyclic ring, or —C(O)(CH₂)_{z}Y wherein z is 0, 1, 2 or 3 and Y is selected from hydrogen, hydroxy, (1-4C)alkoxy, amino, mono(1-6C)alkylamino, di-[(1-6C)alkyl]amino or a saturated or partially saturated 3 to 7 membered heterocyclic ring;

and provided that when R¹ and/or R² is a (1C)alkanoyl group, then the (1C)alkanoyl is not substituted by fluoro or hydroxy;
(nn) R¹ and R² are independently selected from hydrogen, (1-6C)alkanoyl, (1-6C)alkyl, (1-6C)alkoxycarbonyl or (3-6C)cycloalkyl(CH₂)ᵥ— in which v is 0, 1, 2, 3, 4, 5 or 6, or a 5 or 6 membered heteroaryl ring;

wherein the (1-6C)alkyl, the (1-6C)alkanoyl and the (3-6C)cycloalkyl groups are optionally substituted by one or more groups (for example 1 or 2), which may be the same or different, as hereinbefore defined in (mm);

and wherein any heterocyclic and heteroaryl rings within R¹ and/or R² are optionally independently substituted by one or more groups (for example 1 or 2), which may be the same or different, as hereinbefore defined in (mm);
(oo) R¹ and R² are independently selected from hydrogen, (1-6C)alkanoyl, (1-6C)alkyl or a 5 or 6 membered heteroaryl ring;

wherein the (1-6C)alkyl and the (1-6C)alkanoyl groups are optionally substituted by one or more groups (for example 1 or 2), which may be the same or different, as hereinbefore defined in (mm);

and wherein any heterocyclic and heteroaryl rings within R¹ and/or R² are optionally independently substituted by one or more groups (for example 1 or 2), which may be the same or different, as hereinbefore defined in (mm);
(pp) R¹ is hydrogen and R² is selected from hydrogen, (1-6C)alkylsulfonyl, phenyl(CH₂)ᵤ— wherein u is 0, 1, 2, 3, 4, 5 or 6, (1-6C)alkanoyl, (1-6C)alkyl, (1-6C)alkoxycarbonyl, (3-6C)cycloalkyl(CH2)ᵥ— in which v is 0, 1, 2, 3, 4, 5 or 6, or a 5 or 6 membered heteroaryl ring;

wherein the (1-6C)alkyl, the (1-6C)alkanoyl and the (1-6C)cycloalkyl groups are optionally substituted by one or more groups (for example 1 or 2), which may be the same or different, as hereinbefore defined in (mm);

wherein the phenyl is optionally substituted by one or more groups (for example 1 or 2), which may be the same or different, as hereinbefore defined in (mm);

and wherein any heterocyclic and heteroaryl rings within R² are optionally independently substituted by one or more groups (for example 1 or 2), which may be the same or different, as hereinbefore defined in (mm);
(qq) R¹ is hydrogen and R² is selected from hydrogen, (1-6C)alkanoyl, (1-6C)alkyl, (1-6C)alkoxycarbonyl or (3-6C)cycloalkyl(CH₂)ᵥ— in which v is 0, 1, 2, 3, 4, 5 or 6, or a 5 or 6 membered heteroaryl ring;

wherein the (1-6C)alkyl, the (1-6C)alkanoyl and the (3-6C)cycloalkyl groups are optionally substituted by one or more groups (for example 1 or 2), which may be the same or different, as hereinbefore defined in (mm);

and wherein any heterocyclic and heteroaryl rings within R² are optionally independently substituted by one or more groups (for example 1 or 2), which may be the same or different, as hereinbefore defined in (mm);

(rr) R¹ is hydrogen and R² is selected from hydrogen, (1-6C)alkanoyl, (1-6C)alkyl or a 5 or 6 membered heteroaryl ring;

wherein the (1-6C)alkyl and the (1-6C)alkanoyl groups are optionally substituted by one or more groups (for example 1 or 2), which may be the same or different, as hereinbefore defined in (mm);

and wherein any heterocyclic and heteroaryl rings within R² are optionally independently substituted by one or more groups (for example 1 or 2), which may be the same or different, as hereinbefore defined in (mm);
(ss) R¹ and R² are independently selected from hydrogen, (1-6C)alkylsulfonyl, phenyl(CH₂)ᵤ— wherein u is 0, 1, 2, 3, 4, 5 or 6, (1-6C)alkanoyl, (1-6C)alkyl, (1-6C)alkoxycarbonyl, (3-6C)cycloalkyl(CH₂)ᵥ— in which v is 0, 1, 2, 3, 4, 5 or 6, or a 5 or 6 membered heteroaryl ring;

wherein the (1-6C)alkyl, the (1-6C)alkanoyl and the (3-6C)cycloalkyl groups are optionally substituted by one or more groups (for example 1 or 2), which may be the same or different, selected from hydroxy, (1-6C)alkoxy, (1-6C)alkoxy(1-6C)alkoxy, amino, mono(1-6C)alkylamino, di-[(1-6C)alkyl]amino, carbamoyl, mono(1-6C)alkylcarbamoyl, di-[(1-6C)alkyl]carbamoyl or —N(R^{d})C(O)(1-6C)alkyl in which R^{d} is hydrogen or (1-6C)alkyl, or a saturated or partially saturated 3 to 7 membered heterocyclic ring, or a 5 or 6 membered heteroaryl ring, wherein the (1-6C)alkoxy and (1-6C)alkoxy(1-6C)alkoxy groups and the (1-6C)alkyl groups of the mono(1-6C)alkylamino, di-[(1-6C)alkyl]amino, mono(1-6C)alkylcarbamoyl, di-[(1-6C)alkly]carbamoyl and/or —N(R^{d})C(O)(1-6C)alkyl groups are optionally substituted by one or more (for example 1 or 2) hydroxy groups;

wherein the phenyl is optionally substituted by one or more groups (for example 1 or 2), which may be the same or different, selected from halo, (1-6C)alkyl, (1-6C)alkoxy, amino, mono(1-6C)alkylamino or di-[(1-6C)alkyl]amino, wherein the (1-6C)alkyl or (1-6C)alkoxy are optionally substituted by one or more groups (for example 1 or 2), which may be the same or different, selected from hydroxy, amino, mono(1-6C)alkylamino or di-[(1-6C)alkyl]amino;

and wherein any heterocyclic and heteroaryl rings within R¹ and/or R² are optionally independently substituted by one or more groups (for example 1 or 2), which may be the same or different, selected from (1-4C)alkyl, (1-4C)alkoxy, (1-4C)alkoxy(1-4C)alkyl, hydroxy, amino, mono(1-6C)alkylamino or di-[(1-6C)alkyl]amino or a saturated or partially saturated 3 to 7 membered heterocyclic ring, or —C(O)(CH₂)_{z}Y wherein z is 0, 1, 2 or 3 and Y is selected from hydrogen, hydroxy, (1-4C)alkoxy, amino, mono(1-6C)alkylamino, di-[(1-6C)alkyl]amino or a saturated or partially saturated 3 to 7 membered heterocyclic ring;

and provided that when R¹ and/or R² is a (1C)alkanoyl group, then the (1C)alkanoyl is not substituted by fluoro or hydroxy;
(tt) R¹ and R² are independently selected from hydrogen, (1-6C)alkanoyl, (1-6C)alkyl, (1-6C)alkoxycarbonyl or (3-6C)cycloalkyl(CH₂)ᵥ— in which v is 0, 1, 2, 3, 4, 5 or 6, or a 5 or 6 membered heteroaryl ring;

wherein the (1-6C)alkyl, the (1-6C)alkanoyl and the (3-6C)cycloalkyl groups are optionally substituted by one or more groups (for example 1 or 2), which may be the same or different, as hereinbefore defined in (ss);

and wherein any heterocyclic and heteroaryl rings within R¹ and/or R² are optionally independently substituted by one or more groups (for example 1 or 2), which may be the same or different, as hereinbefore defined in (ss);
(uu) R¹ and R² are independently selected from hydrogen, (1-6C)alkanoyl, (1-6C)alkyl, or a 5 or 6 membered heteroaryl ring;

wherein the (1-6C)alkyl and the (1-6C)alkanoyl groups are optionally substituted by one or more groups (for example 1 or 2), which may be the same or different, as hereinbefore defined in (ss);

and wherein any heterocyclic and heteroaryl rings within R¹ and/or R² are optionally independently substituted by one or more groups (for example 1 or 2), which may be the same or different, as hereinbefore defined in (ss);
(vv) R¹ is hydrogen and R² is selected from hydrogen, (1-6C)alkylsulfonyl, phenyl(CH₂)ᵤ— wherein u is 0, 1, 2, 3, 4, 5 or 6, (1-6C)alkanoyl, (1-6C)alkyl, (1-6C)alkoxycarbonyl, (3-6C)cycloalkyl(CH₂)ᵥ— in which v is 0, 1, 2, 3, 4, 5 or 6, or a 5 or 6 membered heteroaryl ring;

wherein the (1-6C)alkyl, the (1-6C)alkanoyl and the (3-6C)cycloalkyl groups are optionally substituted by one or more groups (for example 1 or 2), which may be the same or different, as hereinbefore defined in (ss);

wherein the phenyl is optionally substituted by one or more groups (for example 1 or 2), which may be the same or different, as hereinbefore defined in (ss);

and wherein any heterocyclic and heteroaryl rings within R² are optionally independently substituted by one or more groups (for example 1 or 2), which may be the same or different, as hereinbefore defined in (ss);
(ww) R¹ is hydrogen and R² is selected from hydrogen, (1-6C)alkanoyl, (1-6C)alkyl, (1-6C)alkoxycarbonyl, (3-6C)cycloalkyl(CH₂)ᵥ— in which v is 0, 1, 2, 3, 4, 5 or 6, or a 5 or 6 membered heteroaryl ring;

wherein the (1-6C)alkyl, the (1-6C)alkanoyl and the (3-6C)cycloalkyl groups are optionally substituted by one or more groups (for example 1 or 2), which may be the same or different, as hereinbefore defined in (ss);

and wherein any heterocyclic and heteroaryl rings within R² are optionally independently substituted by one or more groups (for example 1 or 2), which may be the same or different, as hereinbefore defined in (ss);
(xx) R¹ is hydrogen and R² is selected from hydrogen, (1-6C)alkanoyl, (1-6C)alkyl or a 5 or 6 membered heteroaryl ring;

wherein the (1-6C)alkyl and the (1-6C)alkanoyl groups are optionally substituted by one or more groups (for example 1 or 2), which may be the same or different, as hereinbefore defined in (ss);

and wherein any heterocyclic and heteroaryl rings within R² are optionally independently substituted by one or more groups (for example 1 or 2), which may be the same or different, as hereinbefore defined in (ss);
(yy) R¹ is hydrogen and R² is selected from hydrogen, (1-6C)alkanoyl and (1-6C)alkyl;

wherein the (1-6C)alkyl and the (1-6C)alkanoyl groups are optionally substituted by one or more groups (for example 1 or 2), which may be the same or different, as hereinbefore defined in (ss);

and wherein any heterocyclic and heteroaryl rings within R² are optionally independently substituted by one or more groups (for example 1 or 2), which may be the same or different, as hereinbefore defined in (ss);
(zz) R¹ is hydrogen and R² is selected from hydrogen, (1-6C)alkanoyl and (1-6C)alkyl,

wherein the (1-6C)alkyl and the (1-6C)alkanoyl groups are optionally substituted by one or more groups (for example 1 or 2), which may be the same or different, selected from hydroxy, (1-4C)alkoxy, (1-4C)alkoxy(1-4C)alkoxy, amino, mono(1-3C)alkylamino, di(1-3C)alkylamino, carbamoyl or—N(R^{d})C(O)(1-3C)alkyl in which R^{d} is hydrogen or (1-3 C)alkyl, or a saturated 5 or 6 membered heterocyclic ring, or a 5 or 6 membered heteroaryl ring, wherein the (1-4C)alkoxy and (1-4C)alkoxy(1-4C)alkoxy and the (1-3C)alkyl groups of the mono(1-3C)alkylamino, di-[(1-3C)alkyl]amino and/or —N(R^{d})C(O)(1-6C)alkyl groups are optionally substituted by one or more (for example 1 or 2) hydroxy groups;

and wherein any heterocyclic and heteroaryl rings within R² are optionally independently substituted by one or more groups (for example 1 or 2), which may be the same or different, selected from (1-4C)alkyl, (1-4C)alkoxy, (1-4C)alkoxy(1-4C)alkyl, hydroxy, amino, mono(1-3C)alkylamino or di-[(1-3C)alkylamino, or a saturated or partially saturated 3 to 7 membered heterocyclic ring, or —C(O)(CH₂)_{z}Y wherein z is 0, 1, 2 or 3 and Y is selected from hydrogen, hydroxy, (1-4C)alkoxy, amino, mono(1-6C)alkylamino, di-[(1-6C)alkyl]amino or a saturated or partially saturated 3 to 7 membered heterocyclic ring;

and provided that when R¹ and/or R² is a (1C)alkanoyl group, then the (1C)alkanoyl is not substituted by fluoro or hydroxy;
(aaa) R¹ is hydrogen and R² is selected from hydrogen, (1-3C)alkanoyl and (1-3C)alkyl;

wherein the (1-3C)alkyl and the (1-3C)alkanoyl groups are optionally substituted by one or more groups (for example 1 or 2), which may be the same or different, as hereinbefore defined in (zz);

and wherein any heterocyclic and heteroaryl rings within R² are optionally independently substituted by one or more groups (for example 1 or 2), which may be the same or different, as hereinbefore defined in (zz);
(bbb) R¹ is hydrogen and R² is selected from hydrogen and (1-6C)alkyl (particularly (1-3C)alkyl);

wherein the (1-6C)alkyl (particularly (1-3C)alkyl) group is optionally substituted by one or more groups (for example 1 or 2), which may be the same or different, as hereinbefore defined in (zz);

and wherein any heterocyclic and heteroaryl rings within R² are optionally independently substituted by one or more groups (for example 1 or 2), which may be the same or different, as hereinbefore defined in (zz);
(ccc) R¹ is hydrogen and R² is (1-6C)alkyl (particularly (1-3C)alkyl),

wherein the (1-6C)alkyl (particularly (1-3C)alkyl) group is optionally substituted by one or more groups (for example 1 or 2), which may be the same or different, as hereinbefore defined in (zz);

and wherein any heterocyclic and heteroaryl rings within R² are optionally independently substituted by one or more groups (for example 1 or 2), which may be the same or different, as hereinbefore defined in (zz);
(ccc′) R¹ and R² are both hydrogen or R¹ is hydrogen and R² is (1-6C)alkyl

wherein (1-6C)alkyl) is optionally substituted by amino, mono(1-6C)alkylamino or di(1-6C)alkylamino or a saturated 3 to 7 membered heterocyclic ring;
(ccc″) R¹ and R² are both hydrogen or R¹ is hydrogen and R² is (1-6C)alkyl

wherein (1-6C)alkyl) is optionally substituted by di(1-6C)alkylamino or a saturated 3 to 7 membered heterocyclic ring;
(ccc″) R¹ and R² are both hydrogen or R¹ is hydrogen and R² is (1-6C)alkyl

wherein (1-6Calkyl) is optionally substituted by a saturated 3 to 7 membered heterocyclic ring;
(cc′″″) R¹ is hydrogen and R² is selected from hydrogen, and 3-morpholin-4-ylpropyl;
(ccc″″″) R¹ is hydrogen and R² is selected from hydrogen, 3-(dimethylamino)propyl and 3-piperidin-1-ylpropyl;

(ccc′″″″) R¹ is hydrogen and R² is selected from hydrogen, 3-(dimethylamino)propyl and 3-piperidin-1-ylpropyl;
(ddd) NR¹R² is amino.
(ddd′) R¹ and R² are both hydrogen or R¹ is hydrogen or (1-6C)alkyl and R² is (1-6C)alkyl

wherein (1-6Calkyl) is optionally substituted by hydroxy, amino, mono(1-6C)alkylamino or di(1-6C)alkylamino, carbamoyl, (1-6C)alkoxy, (1-6C)alkoxy(1-6C)alkoxy, —N(R^{d})C(O)(1-6C)alkyl in which R^{d} is hydrogen or (1-6C)alkyl, aryl (particularly phenyl), a saturated or partially saturated 3 to 7 membered heterocyclic ring or a 5 or 6 membered heteroaryl ring;

wherein the (1-6C)alkoxy, mono(1-6C)alkylamino and —N(R^{d})C(O)(1-6C)alkyl groups are optionally substituted by hydroxy;

wherein an aryl ring, a saturated or partially saturated 3 to 7 membered heterocyclic ring or a 5 or 6 membered heteroaryl ring is optionally substituted by (1-4C)alkyl, (1-4C)alkoxy or —C(O)CH₂Y wherein Y is selected from hydroxy or di(1-6C)alkylamino.
(eee) R¹ and R² are independently selected from hydrogen, methyl, ethyl, propyl, 2-hydroxyethyl, 3-hydroxypropyl, 2-methoxyethyl, 3-methoxypropyl, 2-aminoethyl, 3-aminopropyl, 2-(isopropylamino)ethyl, 3-(isopropylamino)propyl, 2-(dimethylamino)ethyl, 3-(dimethylamino)propyl, carbamoylmethyl, 2-carbamoylethyl, 3-carbamoylpropyl, 2-(2-methoxyethoxy)acetyl, 2-morpholin-4-ylethyl, 3-morpholin-4-ylpropyl, 2-pyrrolidin-1-ylethyl, 3-pyrrolidin-1-ylpropyl, 3-(4-methylpiperazin-1-yl)propyl, 3-piperidin-1-ylpropyl, 2-piperidin-1-ylethyl, 2-(1H-imidazol-4-yl)ethyl, 2-pyridin-2-ylethyl, 3-(1H-imidazol-1-yl)propyl, 2-pyridin-4-ylethyl, 2,4-dimethoxybenzyl and 5-tert-butylisoxazol-3-yl;
(fff) R¹ is hydrogen and R² is selected from hydrogen, methyl, ethyl, propyl, 2-hydroxyethyl, 3-hydroxypropyl, 2-methoxyethyl, 3-methoxypropyl, 2-aminoethyl, 3-aminopropyl, 2-(isopropylamino)ethyl, 3-(isopropylamino)propyl, 2-(dimethylamino)ethyl, 3-(dimethylamino)propyl, carbamoylmethyl, 2-carbamoylethyl, 3-carbamoylpropyl, 2-(2-methoxyethoxy)acetyl, 2-morpholin-4-ylethyl, 3-morpholin-4-ylpropyl, 2-pyrrolidin-1-ylethyl, 3-pyrrolidin-1-ylpropyl, 3-(4-methylpiperazin-1-yl)propyl, 3-piperidin-1-ylpropyl, 2-piperidin-1-ylethyl, 2-(1H-imidazol-4-yl)ethyl, 2-pyridin-2-ylethyl, 3-(1H-imidazol-1-yl)propyl, 2-pyridin-4-ylethyl, 2,4-dimethoxybenzyl and 5-tert-butylisoxazol-3-yl;
(ggg) R¹ is hydrogen and R² is selected from hydrogen, methyl, ethyl, propyl, 3-(isopropylamino)propyl, 2-pyrrolidin-1-ylethyl, 5-tert-butylisoxazol-3-yl, 3-piperidin-1-ylpropyl, 2-morpholino-4-yl-ethyl, 2-pyrrolidin-1-ylethyl, 3-(dimethylamino)propyl, 2-hydroxyethyl and 2-piperidin-1-ylethyl;
(ggg′) R¹ is hydrogen and R² is selected from R² is (1-6C)alkyl (particularly (1-3C)alkyl), wherein the (1-6C)alkyl (particularly (1-3C)alkyl) group is substituted by a saturated 5 or 6 membered heterocyclic ring;
(ggg″) R¹ is hydrogen and R² is selected from 2-morpholino-4-yl-ethyl or 3-morpholinyl-4-ylpropyl;
(hhh) R² is hydrogen or methyl and R³ is selected from hydrogen, methyl, 2-hydroxyethyl, 2-methoxyethyl, 3-methoxypropyl, 2-(2-hydroxyethoxy)ethyl, 2-methoxyethoxymethyl, 2-aminoethyl, 3-aminopropyl, 4-aminobutyl, 2-(isopropylamino)ethyl, 3-(isopropylamino)propyl, 2-(dimethylamino)ethyl, 3-(dimethylamino)propyl, 4-(dimethylamino)butyl, 2-(dimethylamino)-1-methylethyl, carbamoylmethyl, 2-carbamoylethyl, 2-(2-methoxyethoxy)acetyl, 2-(2-hydroxyacetamido)ethyl, 3-[N-(2-hydroxyethyl)amino]propyl, 2-morpholin-4-ylethyl, 3-morpholin-4-ylpropyl, 2-[(1-methyl-2-morpholin-4-ylethyl), 2-pyrrolidin-1-ylethyl, 3-pyrrolidin-1-ylpropyl, 1-glycoloylpyrrolidin-2-yl)methyl, 1-(N,N-dimethylglycyl)pyrrolidin-2-yl, 2-piperazin-1-ylethyl, 3-piperazin-1-ylpropyl, 3-(4-methylpiperazin-1-yl)propyl, 3-piperidin-1-ylpropyl, 2-(1H-imidazol-4-yl)ethyl, 2-pyridin-2-ylethyl, 3-(1H-imidazol-1-yl)propyl, 5-t-butyl-isoxazol-3-yl, 2-pyridin-4-ylethyl and 2,4-dimethoxybenzyl;
(iii) R¹ is hydrogen and R² is selected from 2-morpholin-4-ylethyl, 3-morpholin-4-ylpropyl, 3-piperidin-1-ylpropyl, 2-piperidin-1-ylethyl, 2-pyrrolidin-1-ylethyl, 4-methyl-piperazin-1-ylpropyl and 3-pyrrolidin-1-ylpropyl;
(jjj) R¹ is hydrogen and R² is selected from 2-morpholin-4-ylethyl, 3-morpholin-4-ylpropyl, 3-piperidin-1-ylpropyl, 2-piperidin-1-ylethyl, 2-pyrrolidin-1-ylethyl, 3-pyrrolidin-1-ylpropyl and 4-methyl-piperazin-1-yl;
(kkk) R¹ and R² are both (1-6C)alkyl (particularly (1-3C)alkyl);
(lll) R¹ is hydrogen and R² is methyl;
(mmm) R¹ and R² are both hydrogen;
(mmm) R³ (including R^{3a} or R^{3b}) is selected from hydrogen, (1-3C)alkyl or (1-3C)alkoxy,

wherein the (1-3C)alkyl and the (1-3C)alkoxy groups are optionally substituted by one or more groups (for example 1 or 2), which may be the same or different, selected from fluoro, hydroxy, (1-6C)alkyl, (1-6C)alkoxy, amino, mono(1-6C)alkylamino, di-[(1-6C)alkyl]amino, carbamoyl, mono(1-6C)alkylcarbamoyl or di-[(1-6C)alkyl]carbamoyl, a saturated or partially saturated 3 to 7 membered heterocyclic ring or a 5 or 6 membered heteroaryl ring, wherein said heterocyclic and heteroaryl rings are optionally independently substituted by one or more groups (for example 1 or 2), which maybe the same or different, selected from (1-4C)alkyl, (1-4C)alkoxy, hydroxy, amino, mono(1-6C)alkylamino or di-[(1-6C)alkyl]amino or a saturated or partially saturated 3 to 7 membered heterocyclic ring;
or R³, R^{3a} or R^{3b} represents a group —NR¹R² as defined above;
(nnn) R³, R^{3a} or R^{3b} is selected from hydrogen or (1-6C)alkyl,

wherein the (1-6C)alkyl group is optionally substituted by one or more groups (for example 1 or 2), which may be the same or different, selected from fluoro, hydroxy, (1-6C)alkyl, (1-6C)alkoxy, amino, mono(1-6C)alkylamino, di-[(1-6C)alkyl]amino, carbamoyl, mono(1-6C)alkylcarbamoyl or di-[(1-6C)alkyl]carbamoyl, a saturated or partially saturated 3 to 7 membered heterocyclic ring or a 5 or 6 membered heteroaryl ring, wherein said heterocyclic and heteroaryl rings are optionally independently substituted by one or more groups (for example 1 or 2), which may be the same or different, selected from (1-4C)alkyl, (1-4C)alkoxy, hydroxy, amino, mono(1-6C)alkylamino or di-[(1-6C)alkyl]amino or a saturated or partially saturated 3 to 7 membered heterocyclic ring; or R³, R^{3a} or R^{3b} represents a group —NR¹R² as defined above;
(ooo) R³, R^{3a} or R^{3b} is selected from hydrogen and a group —NR¹R² as defined above (particularly —NH₂);
(ppp) R³, R^{3a} or R^{3b} is hydrogen;
(qqq) R³, R^{3a} or R^{3b} is a group —NR¹R² as defined above (particularly —NH₂);
(qqq′) R³, R^{3a} or R^{3b} is selected from hydrogen or a group —NR¹R² as defined above (particularly —NH₂);
(qqq″) R³, R^{3a} or R^{3b} is selected from hydrogen or —NH₂.
(rrr) R⁴ is independently selected from hydrogen and (1-6C)alky (particularly (1-3C)alkyl);
(sss) R⁴, R^{4a} or R^{4b} is hydrogen;
(ttt) R³ and R⁴, R^{3a} and R^{4a} or R^{3b} and R^{4b} are both hydrogen;
(uuu) R³, R^{3a} or R^{3b} is a group —NR¹R² as defined above (particularly —NH₂) and R⁴, R^{4a} or R^{4a} is hydrogen;
(uuu′) R⁵ is selected from (1-6C)alkyl and (1-6C)alkoxy;
(uuu″) R⁵ is selected from (1-4C)alkyl and (1-4C)alkoxy;
(uuu′″) R⁵ is selected from methyl and methoxy;
(vvv) n is 0, 1 or 2 (particularly 0 or 1, more particularly 0);
(vvv′) n is 0 or 1;
(www) n is 1 or 2 and R⁵ is independently selected from halo, (1-6C)alkoxy and (1-6C)alkyl, wherein the (1-6C)alkyl and the (1-6C)alkoxy groups are optionally substituted by cyano or one or more fluoro;
(xxx) n is 1 or 2 and R⁵ is independently selected from cyano, halo, (1-6C)alkoxy and (1-6C)alkyl, wherein the (1-6C)alkyl and the (1-6C)alkoxy groups are optionally substituted by cyano or one or more fluoro;
(yyy) n is 0 or 1 and when n is 1, R⁵ is (1-4C)alkyl (particularly methyl);
(zzz) n is 1 or 2 and R⁵ is independently selected from cyclopropyl and (1-6C)alkyl, wherein the (1-6C)alkyl groups are optionally substituted by cyano or one or more fluoro;
(aaaa) n is 1 and R⁵ is (1-6C)alkyl, particularly (1-3C)alkyl;
(bbbb) n is 0
(cccc) n is 1;
(dddd) R⁶ is independently selected from (1-6C)alkyl or (1-6C)alkoxy, (1-6C)alkyl, —S(O)ₚ-(1-6C)alkyl wherein p is 0, 1 or 2, or (1-6C)alkoxy,
wherein the (1-6C)alkyl, —S(O)ₚ-(1-6C)alkyl and the (1-6C)alkoxy(1-6C)alkoxy groups are optionally substituted by one or more groups independently selected from: cyano, fluoro, hydroxy, (1-6C)alkoxy, (1-6C)alkoxy, amino, mono(1-6C)alkylamino, di-[(1-6C)alkyl]amino, di(1-6C)alkylamino, a (3-7C)cycloalkyl ring or a saturated or partially saturated 3 to 7 membered heterocyclic ring; and
wherein the (3-7C)cycloalkyl ring and saturated or partially saturated 3 to 7 membered heterocyclic ring are optionally independently substituted by one or more groups selected from (1-6C)alkyl;

(dddd′) R⁶ is independently selected from halo, cyano, a (3-4C)cycloalkyl ring, a saturated or partially saturated 3 to 7 membered heterocyclic ring or —N(R^{c})C(O)(1-6C)alkyl in which R^{c} is hydrogen or (1-6C)alkyl; or R⁶ is selected from (1-6C)alkyl or (1-6C)alkoxy, wherein the (1-6C)alkyl and the (1-6C)alkoxy groups are optionally substituted by one or more groups (for example 1 or 2), which may be the same or different, selected from cyano, fluoro, hydroxy, (1-6C)alkoxy, amino, mono(1-6C)alkylamino, di-[(1-6C)alkyl]amino, a (3-7C)cycloalkyl ring or a saturated or partially saturated 3 to 7 membered heterocyclic ring;
(eeee) R⁶ is independently selected from halo, cyano, a saturated or partially saturated 3 to 7 membered heterocyclic ring or an —N(R^{c})C(O)(1-6C)alkyl in which R^{c} is hydrogen or (1-6C)alkyl; or R⁶ is selected from (1-6C)alkyl or (1-6C)alkoxy, wherein the (1-6C)alkyl and the (1-6C)alkoxy groups are optionally substituted by one or more groups (for example 1-or 2), which may be the same or different, selected from cyano, fluoro, hydroxy and amino (particularly fluoro);
(ffff) R⁶ is independently selected from halo, cyano, a saturated or partially saturated 3 to 7 membered heterocyclic ring or —N(R^{c})C(O)(1-6C)alkyl in which R^{c} is hydrogen or (1-3C)alkyl; or R⁶ is selected from (1-4C)alkyl or (1-4C)alkoxy, wherein the (1-4C)alkyl and the (1-4C)alkoxy groups are optionally substituted by one or more groups (for example 1 or 2), which may be the same or different, selected from cyano, fluoro, hydroxy and amino (particularly fluoro);
(ggg) R⁶ is selected from fluoro, chloro, cyano, acetylamino, methyl, ethyl, propyl, isopropyl, butyl, tert-butyl, trifluoromethyl, cyclopropyl, cyclopropylmethyl, methoxy, ethoxy, propoxy, butoxy and morpholin-4-yl;
(hhhh) R⁶ is selected from fluoro, chloro, acetylamino, methyl, propyl, tert-butyl, trifluoromethyl, cyclopropyl, cyclopropylmethyl, methoxy and morpholin-4-yl;
(iiii) R⁶ is independently selected from halo or (1-6C)alkyl wherein (1-6C)alkyl is optionally substituted by 1 to 3 halo, particularly fluoro,
(iiii′) R⁶ is independently selected from halo, (1-6C)alkyl, (1-6C)alkoxy, and a saturated 3 to 7 membered heterocyclic ring (particularly morpholin-4-yl)

wherein (1-6C)alkyl is optionally substituted by cyano, a saturated 3 to 7 membered heterocyclic ring or 1 to 3 halo, particularly fluoro, and

wherein any saturated 3 to 7 membered heterocyclic ring within R⁶ is optionally substituted by (1-6C)alkyl
(iiii″) R⁶ is independently selected from (1-6C)alkyl, (1-6C)alkoxy or a saturated 3 to 7 membered heterocyclic ring (particularly morpholin-4-yl or piperidin-1-yl),
wherein (1-6C)alkyl and (1-6C)alkoxy are optionally substituted by 1 to 3 halo, particularly fluoro,
wherein a saturated 3-7 membered heterocyclic ring is optionally substituted by hydroxy(1-2C)alkyl;

(iiii′″) R⁶ is independently selected from hydroxy, halo (particularly chloro or fluoro), (1-6C)alkyl, (1-6C)alkoxy, di-(1-6C)alkylamino or a saturated 3 to 7 membered heterocyclic ring (particularly morpholin-4-yl, piperidin-1-yl or piperazin-1-yl),
wherein (1-6C)alkyl and (1-6C)alkoxy are optionally substituted by 1 to 3 halo, particularly fluoro,
wherein a saturated 3-7 membered heterocyclic ring is optionally substituted by (1-2C)alkyl or hydroxy(1-2C)alkyl;
(iiii″″) R⁶ is independently selected from (1-6C)alkyl (optionally substituted 1 to 3 groups independently selected from halo, particularly fluoro), halo or (1-6C)alkoxy;
(jjjj) R⁶ is independently selected from methyl, t-butyl, fluoro and trifluoromethyl;
(jjjj′) R⁶ is independently selected from methyl, t-butyl, l-cyanoethyl, methoxy, isopropoxy, fluoro, trifluoromethyl, morpholin-4-yl and 4-methylpiperazin-1-ylmethyl;
(jjjj″) R⁶ is independently selected from methyl, trifluoromethyl, morpholin-4-yl or piperidin-1-yl, 4-hydroxymethylpiperidin-1-yl;
(jjjj′″) R⁶ is independently selected from methyl, methoxy, di-methylamino, hydroxy, oxo, chloro, fluoro, trifluoromethyl, morpholin-4-yl or piperidin-1-yl, 4-hydroxymethylpiperidin-1-yl, 4-methylpiperzin-1-yl;
(jjjj″″) R⁶ is independently selected from chloro, fluoro, trifluoromethyl, methyl or methoxy;
(kkkk) R⁶ is independently selected from halo, trifluoromethyl, methyl, tert-butyl, methoxy, acetylamino or morpholino.
(llll) R⁶ is independently selected from halo, cyano, oxo, (3-7C)cycloalkyl, a saturated 3 to 7 membered heterocyclic ring (optionally substituted by (1-4C)alkyl or hydroxy(1-4C)alkyl), —N(R^{c})C(O)(1-6C)alkyl wherein R^{c} is hydrogen or (1-6C)alkyl (particularly (1-4C)alkyl), (1-6C)alkyl (optionally substituted by up to three groups independently selected from halo, particularly fluoro) or (1-6C)alkoxy (optionally substituted by up to three groups independently selected from halo, particularly fluoro).
(llll′) R⁶ is independently selected from hydroxy, halo, cyano, oxo, (3-7C)cycloalkyl, a saturated 3 to 7 membered heterocyclic ring (optionally substituted by (1-4C)alkyl or hydroxy(1-4C)alkyl), —N(R^{c})C(O)(1-6C)alkyl wherein R^{c} is hydrogen or (1-6C)alkyl (particularly (1-4C)alkyl), (1-6C)alkyl (optionally substituted by a saturated 3 to 7 membered heterocyclic ring or up to three groups independently selected from halo, particularly fluoro), (1-6C)alkoxy (optionally substituted by up to three groups independently selected from halo, particularly fluoro) or di-(1-6C)alkylamino;.
(mmmm) R⁶ is independently selected from halo, trifluoromethyl, trifluoromethoxy, cyano, methyl, isopropyl, tert-butyl, methoxy, acetylamino, oxo, cyclopropyl, morpholin-4-yl, piperidin-1-yl, 4-(hydroxymethyl)piperidin-1-yl and 4-methyl-piperazin-1-yl.
(mmmm′) R⁶ is independently selected from hydroxy, halo, trifluoromethyl, trifluoromethoxy, cyano, methyl, isopropyl, tert-butyl, 1-cyanoethyl, methoxy, isopropoxy, di-methylamino, acetylamino, oxo, cyclopropyl, morpholin-4-yl, piperidin-1-yl, 4-(hydroxymethyl)piperidin-1-yl, 4-methyl-piperazin-1-yl and 4-methylpiperazin-1-ylmethyl
(mmmm″) R⁶ is independently selected from halo (such as chloro), trifluoromethyl, methoxy, dimethylamino, morpholin-4-yl or piperidin-1-yl.
(mmmm′″) at least one R⁶ group is selected from amino, mono(C₁₋₆alkyl)amino, di-(C₁₋₆alkyl)amino such as dimethylamino.
(nnnn) m is 1 or 2
(oooo) m is 1;
(pppp) m is 2;
(qqqq) Ring B—R⁶, where m is 1 or 2, is selected from: 2-fluoro-5-trifluoromethylphenyl, 5-t-butylisozazol-3-yl and 2,2-dimethyltetrahydro-2H-pyran-4-yl;
(qqqq′) Ring B—R⁶, where m is 1 or 2, is selected from: phenyl, 3-methoxyphenyl, 3-isopropoxyphenyl, 4-trifluorophenyl, 2-fluoro-5-trifluoromethylphenyl, 3-(1-cyanoethyl)phenyl, 2-morpholin-4-ylphenyl, 3-morpholin-4-ylphenyl, 4-methyl-piperazin-1-ylmethylphenyl, 5-t-butylisozazol-3-yl, 1-methyl-3-tert-butylpyrazol-5-yl. 2,2-dimethyltetrahydro-2H-pyran-4-yl and benzodioxolyl;
(qqqq″) Ring B—R⁶, where m is 1 or 2, is selected from: 2-(trifluoromethyl)phenyl, 2-(trifluoromethoxy)phenyl, 2-oxopyrrolidin-1-yl, 2-morpholin-4-ylphenyl, 2-(piperidin-1-yl)phenyl, 2-[4-(hydroxymethyl)piperidin-1-yl)]phenyl, 5-methyl-furan-2-yl and 4-morpholin-4-ylpyrimidin-5-yl;
(qqqq′″) Ring B—R⁶, where m is 1 or 2, is selected from: 2-hydroxycyclohexyl, 2-methylphenyl, 2-methoxyphenyl, 2-chlorophenyl, 3-chlorophenyl, 2-(dimethylamine)phenyl, 2-(trifluoromethyl)phenyl, 2-(trifluoromethoxy)phenyl, 2-oxopyrrolidin-1-yl, 2-morpholin-4-ylphenyl, 3-morpholin-4-ylphenyl, morpholin-4-yl-5-fluorophenyl, 2-(piperidin-1-yl)phenyl, 2-[4-(hydroxymethyl)piperidin-1-yl)]phenyl, 5-methyl-furan-2-yl, 2-(4-methylpiperzin-1-yl)phenyl and 4-morpholin-4-ylpyrimidin-5-yl;
(qqqq″″) Ring B—R⁶, where m is 1 or 2, is selected from 2-chloro-phenyl, 2,3-dichorophenyl, 2-fluorophenyl, 3,6-di-fluorophenyl, 2-(trifluoromethyl)phenyl, 3-(trifluoromethyl)phenyl, 2-chloro-thien-5-yl, 1-methylimidazol-4-yl, 3-methoxyphenyl and 3,5-dimethyl-isoxazol-4-yl;
(qqqq′″″) Ring B—R⁶, where m is 1 or 2, is selected from 2-chloro-phenyl, 2,3-dichorophenyl, 2-fluorophenyl, 3-fluorophenyl, 4-fluorophenyl, 3,6-di-fluorophenyl, 2-(trifluoromethyl)phenyl, 3-(trifluoromethyl)phenyl, 2-chloro-thien-5-yl, 1-methylimidazol-4-yl, 3-methoxyphenyl and 3,5-dimethyl-isoxazol-4-yl;
(rrrr) Ring B—R⁶, where m is 1 or 2, is selected from 2-fluoro-phenyl, 3-fluoro-phenyl, 4-fluoro-phenyl, 2-chloro-phenyl, 3-chloro-phenyl, 2,3-dichloro-phenyl, 3,4-dichloro-phenyl, 2,5-difluoro-phenyl, 3,4-difluoro-phenyl, 4,5-difluoro-phenyl, 3,6-di-fluorophenyl, 2-methoxyphenyl, 3-methoxyphenyl, 4-methoxyphenyl, 3-cyano-phenyl, 2-(trifluoromethyl)phenyl, 2-(trifluoromethoxy)phenyl, 3-(trifluoromethyl)phenyl, 4-(trifluoromethyl)phenyl, 2-fluoro-5-(trifluoromethyl)phenyl, 3-acetylaminophenyl, 2-morpholin-4-ylphenyl, 3-fluoro-5-(4-methylpiperazin-1-yl)phenyl, 2-morpholin-4-ylphenyl, 2-(piperidin-1-yl)phenyl, 2-(4-hydroxymethylpiperidin-1-yl)phenyl, 2-oxopyrrolidin-1-yl, 2-oxo-piperidin-3-yl, 1-methylpiperidin-4-yl, 1-propylpiperidin-4-yl, 2,2-dimethyltetrahydropyran-4-yl, 5-methyl-furan-2-yl, 5-methyl-1,3,4-thiadiazol-2-yl, 5-t-butyl-1,3,4-thiadiazol-2-yl, 5-triflluoromethyl-1,3,4-thiadiazol-2-yl, 5-cyclopropyl-1,3,4-thiadiazol-2-yl, 5-ethyl-1,3,4-thiadiazol-2-yl, 5-isopropyl-1,3,4-thiadiazol-2-yl, 5-ethylthio-1,3,4-thiadiazol-2-yl, 3-methylisoxazol-5-yl, 4-methyl-isoxazol-3-yl, 5-methylisoxazol-3-yl, 5-t-butyl-isoxazol-3-yl, 3,5-dimethylisoxazol-4-yl,4-t-butyl-thiazol-2-yl, 3-methyl-isothiazol-5-yl, 4-methyl-isothiazol-2-yl, 1-methyl-1 H-imidazol-4-yl, 2-chloro-thien-5-yl1-methyl-3-t-butyl-pyrazol-5-yl, 1-methyl-3-cyclopropyl-pyrazol-5-yl, 1-methyl-3-isopropyl-pyrazol-5-yl, 1-t-butyl-pyrazol-4-yl, 1-t-butyl-3-cyclopropyl-pyrazol-5-yl, 1-ethyl-pyrazol-3-yl, 1-isopropyl-pyrazol-3-yl, 5-isopropyl-1,3,4-oxadiazol-2-yl, 4-trifluoro-pyrid-2-yl, 4-(trifluoromethyl)pyrid-3-yl and 4-(trifluoromethyl)pyrid-2-yl;5-methylpyrazin-2-yl and 4-morpholin-4-ylpyrimidin-5-yl;
(rrrr) Ring B—R⁶, where m is 1 or 2, is selected from
2-hydroxycyclohexyl,phenyl, 2-methylphenyl, 2-fluoro-phenyl, 3-fluoro-phenyl, 4-fluoro-phenyl, 2-chloro-phenyl, 3-chloro-phenyl, 2,3-dichloro-phenyl, 3,4-dichloro-phenyl, 2,5-difluoro-phenyl, 3,4-difluoro-phenyl, 4,5-difluoro-phenyl, 3,6-di-fluorophenyl, 2-methylphenyl, 2-methoxyphenyl, 3-methoxyphenyl, 4-methoxyphenyl, 3-isopropoxyphenyl, 3-cyano-phenyl, 3-(1-cyanoethyl)phenyl, 2-(trifluoromethyl)phenyl, 2-(trifluoromethoxy)phenyl, 3-(trifluoromethyl)phenyl, 4-(trifluoromethyl)phenyl, 2-fluoro-5-(trifluoromethyl)phenyl, 2-(dimethylamine)phenyl, 3-acetylaminophenyl, 2-morpholin-4-ylphenyl, 3-morpholin-4-ylphenyl, 2-morpholin-4-yl-5-fluorophenyl, 2-(4-methylpiperazin-1-yl)phenyl, 3-fluoro-5-(4-methylpiperazin-1-yl)phenyl, 2-(piperidin-1-yl)phenyl, 2-(4-hydroxymethylpiperidin-1-yl)phenyl, 2-oxopyrrolidin-1-yl, 2-oxo-piperidin-3-yl, 1-methylpiperidin-4-yl, 1-propylpiperidin-4-yl, 4-methyl-piperazin-1-ylmethylphenyl, 2,2-dimethyltetrahydropyran-4-yl, 5-methyl-furan-2-yl, 5-methyl-1,3,4-thiadiazol-2-yl, 5-t-butyl-1,3,4-thiadiazol-2-yl, 5-trifluoromethyl-1,3,4-thiadiazol-2-yl, 5-cyclopropyl-1,3,4-thiadiazol-2-yl, 5-ethyl-1,3,4-thiadiazol-2-yl, 5-isopropyl-1,3,4-thiadiazol-2-yl, 5-ethylthio-1,3,4-thiadiazol-2-yl, 3-methylisoxazol-5-yl, 4-methyl-isoxazol-3-yl, 5-methylisoxazol-3-yl, 5-t-butyl-isoxazol-3-yl, 3,5-dimethylisoxazol-4-yl, 4-t-butyl-thiazol-2-yl, 3-methyl-isothiazol-5-yl, 4-methyl-isothiazol-2-yl, 1-methyl-1H-imidazol-4-yl, 2-chloro-thien-5-yl1-methyl-3-t-butyyl-pyrazol-5-yl, 1-methyl-3-cyclopropyl-pyrazol-5-yl, 1-methyl-3-isopropyl-pyrazol-5-yl, 1-t-butyl-pyrazol-4-yl, 1-t-butyl-3-cyclopropyl-pyrazol-5-yl, 1-ethyl-pyrazol-3-yl, 1-isopropyl-pyrazol-3-yl, 5-isopropyl-1,3,4-oxadiazol-2-yl, 4-trifluoro-pyrid-2-yl, 4-(trifluoromethyl)pyrid-3-yl and 4-(trifluoromethyl)pyrid-2-yl; 5-methylpyrazin-2-yl and 4-morpholin-4-ylpyrimidin-5-yl;benzodioxolyl;
(rrrr″) Ring B—R⁶, where m is 1 or 2, is selected from
2-hydroxycyclohexyl, phenyl, 2-methylphenyl, 2-fluoro-phenyl, 3-fluoro-phenyl, 4-fluoro-phenyl, 2-chloro-phenyl, 3-chloro-phenyl, 2,3-dichloro-phenyl, 3,4-dichloro-phenyl, 2,5-difluoro-phenyl, 3,4-difluoro-phenyl, 4,5-difluoro-phenyl, 3,6-di-fluorophenyl, 2-methylphenyl, 2-methoxyphenyl, 3-methoxyphenyl, 4-methoxyphenyl, 3-isopropoxyphenyl, 3-cyano-phenyl, 3-(1-cyanoethyl)phenyl, 2-(trifluoromethyl)phenyl, 2-(trifluoromethoxy)phenyl, 3-(trifluoromethyl)phenyl, 4-(trifluoromethyl)phenyl, 2-fluoro-5-(trifluoromethyl)phenyl, 2-(dimethylamine)phenyl, 3-acetylaminophenyl, 2-morpholin-4-ylphenyl, 3-morpholin-4-ylphenyl, 2-morpholin-4-yl-5-fluorophenyl, 2-(4-methylpiperazin-1-yl)phenyl, 3-fluoro-5-(4-methylpiperazin-1-yl)phenyl, 2-(piperidin-1-yl)phenyl, 2-(4-hydroxymethylpiperidin-1-yl)phenyl, 2-oxopyrrolidin-1-yl, 2-oxo-piperidin-3-yl, 1-methylpiperidin-4-yl, 1-propylpiperidin-4-yl, 4-methyl-piperazin-1-ylmethylphenyl, 2,2-dimethyltetrahydropyran-4-yl, 5-methyl-furan-2-yl, 5-methyl-1,3,4-thiadiazol-2-yl, 5-t-butyl-1,3,4-thiadiazol-2-yl, 5-triflluoromethyl-1,3,4-thiadiazol-2-yl, 5-cyclopropyl-1,3,4-thiadiazol-2-yl, 5-ethyl-1,3,4-thiadiazol-2-yl, 5-isopropyl-1,3,4-thiadiazol-2-yl, 5-ethylthio-1,3,4-thiadiazol-2-yl, 3-methylisoxazol-5-yl, 4-methyl-isoxazol-3-yl, 5-methylisoxazol-3-yl, 5-t-butyl-isoxazol-3-yl, 3,5-dimethylisoxazol-4-yl, 4-t-butyl-thiazol-2-yl, 3-methyl-isothiazol-5-yl, 4-methyl-isothiazol-2-yl, 1-methyl-1H-imidazol-4-yl, 2-chloro-thien-5-yl, 1-methyl-3-t-butyl-pyrazol-5-yl, 1-methyl-3-cyclopropyl-pyrazol-5-yl, 1-methyl-3-isopropyl-pyrazol-5-yl, 1-t-butyl-pyrazol-4-yl, 1-t-butyl-3-cyclopropyl-pyrazol-5-yl, 1-ethyl-pyrazol-3-yl, 1-isopropyl-pyrazol-3-yl, 5-isopropyl-1,3,4-oxadiazol-2-yl, 4-trifluoro-pyrid-2-yl, 4-(trifluoromethyl)pyrid-3-yl and 4-(trifluoromethyl)pyrid-2-yl; 5-methylpyrazin-2-yl, 4-morpholin-4-ylpyrimidin-5-yl; benzodioxolyl, and 2-(dimethylamino)phenyl;
(ssss) A is selected from phenyl, furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl and 1,3,5-triazinyl (particularly phenyl, thiazolyl, thiadiazolyl, pyridyl and pyrimidinyl);

n is 0; and

L is attached meta or para on ring A with respect to the point of attachment of the ethynyl group and represents —N(R⁸)C(O)—(CR^{a}R^{b})ₓ-Z-(CR^{a}R^{b})_{y}—, —C(O)N(R⁹)—(CR^{a}R^{b})ₓ-Z-(CR^{a}R^{b})_{y}—, —C(R^{a})(R^{b})N(R⁸)C(O)—(CR^{a}R^{b})ₓ-Z-(CR^{a}R^{b})_{y}— or —C(R^{a})(R^{b})C(O)N(R⁹)—(CR^{a}R^{b})ₓ-Z-(CR^{a}R^{b})_{y}— wherein Z is —O— or —N(R⁸)— or L represents —C(O)N(R⁹)—CH₂— or L represents —C(O)N(R⁹)— or —N(R⁸)C(O)—;

R⁸, R⁹, R^{a} and R^{b} independently represent hydrogen or (1-6C)alkyl (particularly hydrogen or (1-3C)alkyl, more particularly hydrogen);

x and y are independently 0, 1, or 2, with the proviso that x+y>0 and x+y<3,
(tttt) A is selected from phenyl, furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl and 1,3,5-triazinyl (particularly phenyl, thiazolyl, thiadiazolyl, pyridyl and pyrimidinyl);

n is 0; and

L is attached meta or para on ring A with respect to the point of attachment of the ethynyl group and represents —N(R⁸)C(O)—(CR^{a}R^{b})ₓ-Z-(CR^{a}R^{b})_{y}—, —C(O)N(R⁹)—(CR^{a}R^{b})ₓ-Z-(CR^{a}R^{b})_{y}—, —C(R^{a})(R^{b})N(R⁸)C(O)—(CR^{a}R^{b})ₓ-Z-(CR^{a}R^{b})_{y}— or —C(R^{a})(R^{b})C(O)N(R⁹)—(CR^{a}R^{b})ₓ-Z-(CR^{a}R^{b})_{y}— wherein Z is —O— or —N(R⁸)— or L represents —C(O)N(R⁹)—CH₂— or L represents —C(O)N(R⁹)— or —N(R⁸)C(O)—;

R⁸, R⁹, R^{a} and R^{b} independently represent hydrogen or (1-6C)alkyl (particularly hydrogen or (1-3C)alkyl, more particularly hydrogen);

x and y are independently 0, 1, or 2, with the proviso that x+y>0 and x+y<3,
(uuuu) A is phenyl;

n is 0; and

B is selected from a saturated or partially saturated 4 to 6 membered heterocyclic ring, an aryl group, a 5 or 6 membered heteroaryl ring selected from furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl or 1,3,5-triazinyl or a 8, 9 or 10 membered bicyclic group which optionally contains 1, 2, 3 or 4 heteroatoms independently selected from N, O and S and which is saturated, partially saturated or aromatic;
(vvvv) A is phenyl;

n is 0; and

B is selected from phenyl, pyrazolyl, thiadiazolyl and isoxazolyl;
(wwww) A is selected from phenyl, furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl and 1,3,5-triazinyl (particularly phenyl, thiazolyl, thiadiazolyl, pyridyl and pyrimidinyl);

n is 0;

L is attached meta or para on ring A with respect to the point of attachment of the ethynyl group and represents —N(R⁸)C(O)—(CR^{a}R^{b})ₓ-Z-(CR^{a}R^{b})_{y}—, —C(O)N(R⁹)—(CR^{a}R^{b})ₓ-Z-(CR^{a}R^{b})_{y}—, —C(R^{a})(R^{b})N(R⁸)C(O)—(CR^{a}R^{b})ₓ-Z-(CR^{a}R^{b})_{y}— or —C(R^{a})(R^{b})C(O)N(R⁹)—(CR^{a}R^{b})ₓ-Z-(CR^{a}R^{b})_{y}— wherein Z is —O— or —N(R⁸)— or L represents —C(O)N(R⁹)—CH₂— or L represents —C(O)N(R⁹)— or —N(R⁸)C(O)—;

R⁸, R⁹, R^{a} and R^{b} independently represent hydrogen or (1-6C)alkyl (particularly hydrogen or (1-3C)alkyl, more particularly hydrogen);

x and y are independently 0, 1, or 2, with the proviso that x+y>0 and x+y<3,

B is selected from phenyl, pyrazolyl, thiadiazolyl and isoxazolyl;
(xxxx) A is selected from phenyl, furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl and 1,3,5-triazinyl (particularly phenyl, thiazolyl, thiadiazolyl, pyridyl and pyrimidinyl);

n is 0;

L is attached meta or para on ring A with respect to the point of attachment of the ethynyl group and represents —N(R⁸)C(O)—(CR^{a}R^{b})ₓ-Z-(CR^{a}R^{b})_{y}—, —C(O)N(R⁹)—(CR^{a}R^{b})ₓ-Z-(CR^{a}R^{b})_{y}—, —C(R^{a})(R^{b})N(R⁸)C(O)—(CR^{a}R^{b})ₓ-Z-(CR^{a}R^{b})_{y}— or —C(R^{a})(R^{b})C(O)N(R⁹)—(CR^{a}R^{b})ₓ-Z-(CR^{a}R^{b})_{y}— wherein Z is —O— or —N(R⁸)— or L represents —C(O)N(R⁹)—CH₂— or L represents —C(O)N(R⁹)— or —N(R⁸)C(O)—;

R⁸, R⁹, R^{a} and R^{b} independently represent hydrogen or (1-6C)alkyl (particularly hydrogen or (1-3C)alkyl, more particularly hydrogen);

x and y are independently 0, 1, or 2, with the proviso that x+y>0 and x+y<3,

B is selected from phenyl, pyrazolyl, thiadiazolyl and isoxazolyl;
(yyyy) m is 0, 1 or 2 (particularly 1 or 2);
(zzzz) B is selected from cyclopentyl, cyclohexyl, piperidinyl, tetrahydropyranyl, phenyl, furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazinyl, 2,3-dihydro-1,4-benzodioxinyl and 1,3-benzodioxol-5-yl;

m is 1 or 2; and

R⁶ is independently selected from fluoro, chloro, cyano, acetylamino, methyl, ethyl, propyl, isopropyl, butyl, tert-butyl, trifluoromethyl, cyclopropyl, cyclopropylmethyl, methoxy, ethoxy, propoxy, butoxy and morpholin-4-yl;
(aaaaa) B is selected from phenyl, isoxazolyl, isothiazolyl, thiadiazolyl, pyrazolyl and pyridyl;

m is 1 or 2; and

R⁶ is independently selected from halo, cyano, a (3-4C)cycloalkyl ring, a saturated or partially saturated 3 to 7 membered heterocyclic ring or —N(R^{c})C(O)(1-6C)alkyl in which R^{c} is hydrogen or (1-6C)alkyl; or R⁶ is selected from (1-6C)alkyl or (1-6C)alkoxy, wherein the (1-6C)alkyl and the (1-6C)alkoxy groups are optionally substituted by one or more groups (for example 1 or 2), which may be the same or different, selected from cyano, fluoro, hydroxy and amino (particularly fluoro);
(bbbbb) B is selected from phenyl, isoxazolyl, isothiazolyl, thiadiazolyl, pyrazolyl and pyridyl;

m is 1 or 2; and

R⁶ is independently selected from fluoro, chloro, cyano, acetylamino, methyl, ethyl, propyl, isopropyl, butyl, tert-butyl, trifluoromethyl, cyclopropyl, cyclopropylmethyl, methoxy, ethoxy, propoxy, butoxy and morpholin-4-yl;
(ccccc) B is phenyl;

m is 1 or 2; and

R⁶ is independently selected from fluoro, chloro, cyano, acetylamino, trifluoromethyl, cyclopropyl, cyclopropylmethyl, methoxy, ethoxy, propoxy, butoxy and morpholin-4-yl;
(ddddd) B is phenyl;

m is 1 or 2; and

R⁶ is independently selected from fluoro and trifluoromethyl;
(eeeee) B is isoxazolyl;

m is 1 or 2; and

R⁶ is independently selected from fluoro, chloro, cyano, acetylamino, methyl, ethyl, propyl, isopropyl, butyl, tert-butyl, trifluoromethyl, cyclopropyl, cyclopropylmethyl, methoxy, ethoxy, propoxy and butoxy;
(fffff) B is isoxazolyl;

m is 1 or 2; and

R⁶ is independently selected from methyl, ethyl, propyl, isopropyl, butyl, tert-butyl (particularly methyl and tert-butyl, more particularly tert-butyl);
(ggggg) B is pyrazolyl;

m is 1 or 2; and

R⁶ is independently selected from fluoro, chloro, cyano, acetylamino, methyl, ethyl, propyl, isopropyl, butyl, tert-butyl, trifluoromethyl, cyclopropyl, cyclopropylmethyl, methoxy, ethoxy, propoxy and butoxy;
(hhhhh) B is pyrazolyl;

m is 1 or 2; and

R⁶ is independently selected from methyl, ethyl, propyl, isopropyl, butyl, tert-butyl (particularly methyl and tert-butyl, more particularly tert-butyl);
(iiiii) B is thiadiazolyl;

m is 1 or 2; and

R⁶ is independently selected from fluoro, chloro, cyano, acetylamino, methyl, ethyl, propyl, isopropyl, butyl, tert-butyl, trifluoromethyl, cyclopropyl, methoxy, ethoxy, propoxy and butoxy;
(jjjj) B is thiadiazolyl;

m is 1 or 2; and

R⁶ is independently selected from methyl, ethyl, propyl, isopropyl, butyl, tert-butyl (particularly methyl and tert-butyl, more particularly tert-butyl);
(cccc) Ring B—R⁶ wherein m is 0, 1 or 2 is selected from cyclopropyl, cyclobutyl, cyclopentyl, cyclohexyl, cycloheptyl, phenyl, 2-fluorophenyl, 3-fluorophenyl, 4-fluorophenyl, 2,5-difluorophenyl, 3,4-difluorophenyl, 3,5-difluorophenyl, 2-(trifluoromethyl)phenyl, 3-(trifluoromethyl)phenyl, 4-(trifluoromethyl)phenyl, 2-fluoro-5-(trifluoromethyl)phenyl, 2-chlorophenyl, 3-chlorophenyl, 4-chlorophenyl, 2,5-dichlorophenyl, 3,4-dichlorophenyl, 3,5-dichlorophenyl, 2-methoxyphenyl, 3-methoxyphenyl, 4-methoxyphenyl, 2-cyanophenyl, 3-cyanophenyl, 4-cyanophenyl, 2-acetamidophenyl, 3-acetamidophenyl, 4-acetamidophenyl, 5-tert-butyl-1,3,4-thiadizol-2-yl, 5-methyl-1,3,4-thiadizol-2-yl, 5-cyclopropyl-1,3,4-thiadizol-2-yl, 1-methyl-3-cyclopropyl-pyrazol-5-yl, 1-tert-butyl-3-cyclopropyl-pyrazol-5-yl, 3-methylisothiazol-5-yl, 3-methylisoxazol-5-yl, 5-methylisoxazol-3-yl, 5-tert-butylisoxazol-3-yl, 4-(trifluoromethyl)pyridin-2-yl, 2-oxopiperidin-3-yl, 2,2-dimethyltetrahydro-2H-pyran-4-yl, 2,3-dihydro-1,4-benzodioxinyl, 1,3-benzodioxol-5-yl, 2-morpholin-4-ylphenyl, 3-morpholin-4-ylphenyl, 4-morpholin-4-ylphenyl, 1-methylpiperidin-4-yl, 1-ethylpiperidin-4-yl and 1-propylpiperidin-4-yl; and
(kkkkk) Ring B—R⁶ wherein m is 1 or 2 is selected from 2-fluorophenyl, 3-fluorophenyl, 4-fluorophenyl, 2,5-difluorophenyl, 3,4-difluorophenyl, 3,5-difluorophenyl, 2-(trifluoromethyl)phenyl, 3-(trifluoromethyl)phenyl, 4-(trifluoromethyl)phenyl, 2-fluoro-5-(trifluoromethyl)phenyl, 2-chlorophenyl, 3-chlorophenyl, 4-chlorophenyl, 2,5-dichlorophenyl, 3,4-dichlorophenyl, 3,5-dichlorophenyl, 2-methoxyphenyl, 3-methoxyphenyl, 4-methoxyphenyl, 2-cyanophenyl, 3-cyanophenyl, 4-cyanophenyl, 2-acetamidophenyl, 3-acetamidophenyl, 4-acetamidophenyl, 5-tert-butyl-1,3,4-thiadizol-2-yl, 5-methyl-1,3,4-thiadizol-2-yl, 5-cyclopropyl-1,3,4-thiadizol-2-yl, 3-cyclopropyl-pyrazol-5-yl, 1-tert-butyl-3-cyclopropyl-pyrazol-5-yl, 3-methylisothiazol-5-yl, 3-methylisoxazol-5-yl, 5-methylisoxazol-3-yl, 5-tert-butylisoxazol-3-yl, 4-(trifluoromethyl)pyridin-2-yl, 2-oxopiperidin-3-yl, 2,2-dimethyltetrahydro-2H-pyran-4-yl, 2-morpholin-4-ylphenyl, 3-morpholin-4-ylphenyl, 4-morpholin-4-ylphenyl, 1-methylpiperidin-4-yl, 1-ethylpiperidin-4-yl and 1-propylpiperidin-4-yl.
(lllll) R¹ and R² are both hydrogen, R³ and R⁴ are both hydrogen, n is 0, L is —C(O)NH— or —NHC(O)— and ring B—R⁶, where m is 1 or 2, is selected from 3-acetylaminophenyl, 2-(trifluoromethyl)phenyl, 3-(trifluoromethyl)phenyl, 4-(trifluoromethyl)phenyl, 2-methoxyphenyl, 3-methoxyphenyl, 2-fluoro-5-(trifluoromethyl)phenyl, 3,4-dichloro-phenyl, 2-morpholin-4-ylphenyl, 5-tert-butyl-1,3,4-thiadiazol-2-yl, 3-methylisothiazol-5-yl, 3-methylisoxazol-5-yl, 5-tert-butylisoxazol-3-yl, 1-methyl-3-tert-butyl-pyrazol-5-yl, 1-methylpiperidin-4-yl, 1-propylpiperidin-4-yl, 4-(trifluoromethyl)pyrid-3-yl and 4-(trifluoromethyl)pyrid-2-yl;

A particular embodiment of the compounds of the Formula I is a compound of the Formula IA(i):

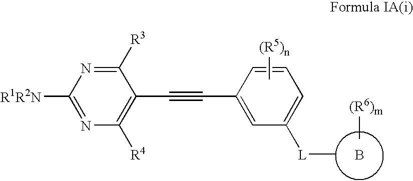

wherein R¹, R², R³, R⁴, R⁵, R⁶, L, B, n and m are as defined above and salts thereof, particularly pharmaceutically acceptable salts thereof.

Another particular embodiment of the compounds of the Formula I is a compound of the Formula IA(ii):

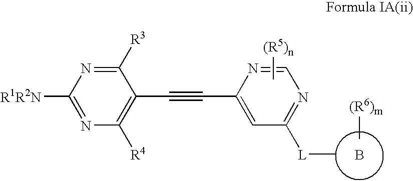

wherein R¹, R², R³, R⁴, R⁵, R⁶, L, B, n and m are as defined above and salts thereof, particularly pharmaceutically acceptable salts thereof.

Another particular embodiment of the compounds of the Formula I is a compound of the Formula IA(iii):

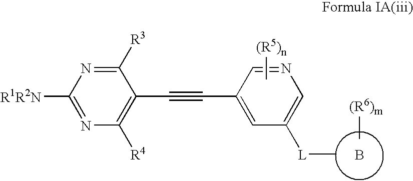

wherein R¹, R², R³, R⁴, R⁵, R⁶, L, B, n and m are as defined above and salts thereof, particularly pharmaceutically acceptable salts thereof.

Another particular embodiment of the compounds of the Formula I is a compound of the Formula IA(iv):

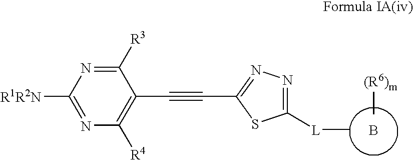

wherein R¹, R², R³, R⁴, R⁵, R⁶, L, B, n and m are as defined above and salts thereof, particularly pharmaceutically acceptable salts thereof.

A particular embodiment of the compounds of the Formula IB is a compound of the Formula IB(i):

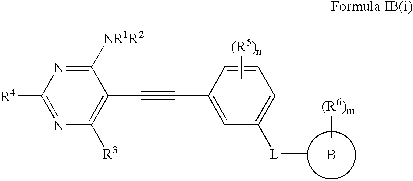

wherein R¹, R², R³, R⁴, R⁵, R⁶, L, B, n and m are as defined above and salts thereof, particularity pharmaceutically acceptable salts thereof.

Another particular embodiment of the compounds of the Formula IB is a compound of the Formula IB(ii):

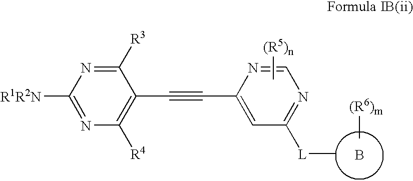

wherein R¹, R², R³, R⁴, R⁵, R⁶, L, B, n and m are as defined above and salts thereof, particularity pharmaceutically acceptable salts thereof.

Another particular embodiment of the compounds of the Formula IB is a compound of the Formula IB(iii):

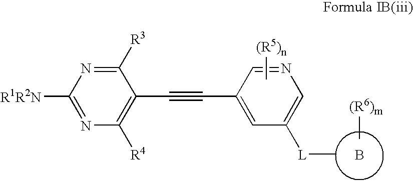

wherein:
wherein R¹, R², R³, R⁴, R⁵, R⁶, L, B, n and m are as defined above and salts thereof, particularity pharmaceutically acceptable salts thereof.

Another particular embodiment of the compounds of the Formula I is a compound of the Formula IB(iv):

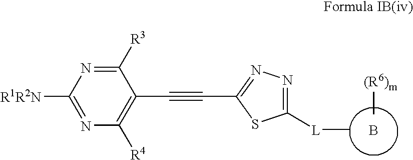

wherein R¹, R², R³, R⁴, R⁵, R⁶, L, B, n and m are as defined above and salts thereof, particularity pharmaceutically acceptable salts thereof.

A compound of the Formula I, or a pharmaceutically-acceptable salt thereof, may be prepared by any process known to be applicable to the preparation of chemically-related compounds. Such processes, when used to prepare a compound of the Formula I are provided as a further feature of the invention and are illustrated by the following representative process variants. Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated which are within the ordinary skill of an organic chemist.

According to a further aspect of the present invention provides a process for preparing a compound of formula I or a pharmaceutically acceptable salt thereof (wherein R¹, R², R³, R⁴, R⁵, R⁶, R⁷, R⁸, R⁹, R¹⁰, R¹¹ L, ring A and ring B, n and m are, unless otherwise specified, as defined in formula I) as described schematically below.
Process (a) For compounds of the formula I wherein L is —N(R⁸)C(O)—(CR^{a}R^{b})ₓ-Z-(CR^{a}R^{b})_{y}— or —C(CR^{a}R^{b})N(R⁸(C(O)—(CR^{a}R^{b})ₓ-Z-(CR^{a}R^{b})_{y}—, the reaction of a compound of the formula II:

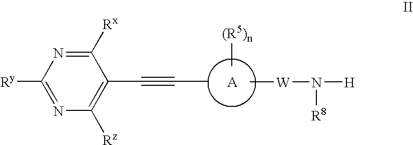

wherein W is —C(R^{a}R^{b})— or a direct bond and R^{x}, R^{y}, R^{z}, R⁵, R⁸, R^{a}, R^{b}, n, p and A have any of the meanings defined hereinbefore except that any functional group is protected if necessary, with a heterocycle of the formula III:

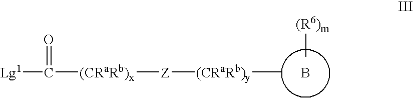

wherein Lg¹ is a suitable displaceable group for example hydroxy, halogeno (such as fluoro, chloro or bromo), R^{x}—C(O)—O— or R^{x}—O— (wherein R^{x} is a suitable alkyl or aryl group) and R⁶, R^{a}, R^{b}, m, x, y and B have any of the meanings defined hereinbefore except that any functional group is protected if necessary; or
Process (b) For compounds of the formula I wherein L is —C(R^{a}R^{b})C(O)N(R⁹)—(CR^{a}R^{b})ₓ-Z-(CR^{a}R^{b})_{y}— or —C(O)N(R⁹)—(CR^{a}R^{b})ₓ-Z-(CR^{a}R^{b})_{y}—, the reaction of a compound of the formula IV:

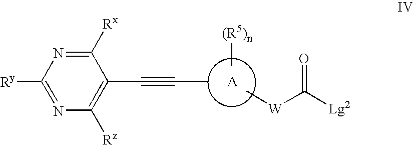

wherein Lg² is a suitable displaceable group as described above for Lg¹, W is —C(R^{a}R^{b})— or a direct bond and R^{x}, R^{y}, R^{z}, R⁵, R^{a}, R^{b}, n, p and A have any of the meanings defined hereinbefore except that any functional group is protected if necessary, with an amine of the formula V:

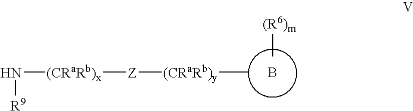

wherein R⁶, R⁹, R^{a}, R^{b}, m, x, y, B and Z have any of the meanings defined hereinbefore except that any functional group is protected if necessary; or
Process (c) For compounds of formula I wherein Z is —O— or —N(R^{a})— and when Z is —O— then x>0, the reaction of a compound of formula VI

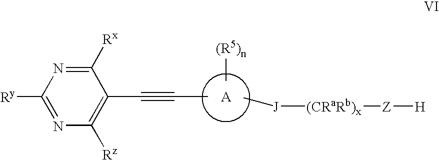

wherein J is selected from —N(R⁸)C(O)—, —C(O)N(R⁹)—, —C(R^{a})(R^{b})—N(R⁸)C(O)— or —C(R^{a})(R^{b})—C(O)N(R⁹)— and Z R^{x}, R^{y}, R^{z}, R⁵, R⁸, R⁹, R^{a}, R^{b}, x, n and A have any of the meanings defined hereinbefore except that any functional group is protected if necessary and when Z is —O— then x>0, with a compound of formula VII,

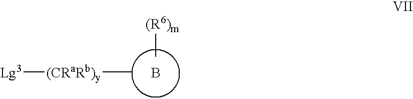

wherein Lg³is a suitable displaceable group, for example halogeno (such as fluoro, chloro, bromo), O-tosyl, O-mesyl or trifluorosulphonyloxy and R^{a}, R^{b}, R⁶, y, m and B have any of the meanings defined hereinbefore except that any functional group is protected if necessary;
Process (d) For compounds of formula I wherein Z is —O— or —N(R^{a})— and x>0, the reaction of a compound of formula VIII

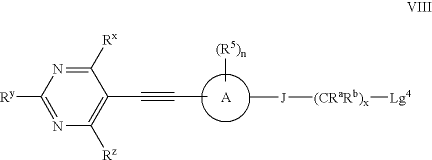

wherein Lg⁴ is a suitable displaceable group, for example halogeno (such as chloro, bromo), O-tosyl, O-mesyl or trifluorosulphonyloxy, J is selected from —N(R⁸)C(O)—, —C(O)N(R⁹)—, —C(R^{a})(R^{b})—N(R⁸)C(O)— or —C(R^{a})(R^{b})—C(O)N(R⁹)— and R^{x}, R^{y}, R^{z}, R⁵, R⁸, R⁹, R^{a}, R^{b}, n, and A have any of the meanings defined hereinbefore except that any functional group is protected if necessary and x is 1, 2 or 3, with a compound of formula IX,

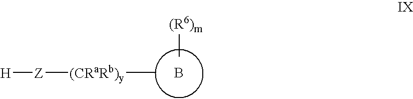

wherein Z, R^{a}, R^{b}, R⁶, y, m and B have any of the meanings defined hereinbefore except that any functional group is protected if necessary; or
Process (e) For the preparation of a compound of formula IA, the reaction of a compound of the formula X:

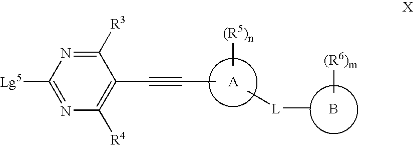

wherein Lg⁵ is a suitable displaceable group for example halogeno (such as fluoro, chloro, bromo or iodo), methyl sulfonyl, methylthio, methylsulphoxide or aryloxy (such as phenoxy) and R³, R⁴, R⁵, R⁶, n, m, A, B and L have any of the meanings defined hereinbefore except that any functional group is protected if necessary, with an amine of the formula HNR¹R², wherein R¹ and R² have any of the meanings defined hereinbefore except that any functional group is protected if necessary;
or for the preparation of a compound of formula (IB), the reaction of a compound of the formula X

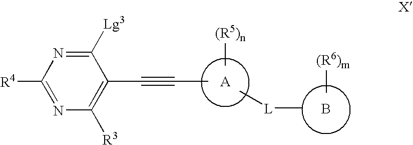

wherein Lg³ is a suitable displaceable group for example halogeno (such as fluoro, chloro, bromo or iodo), methyl sulfonyl, methylthio or aryloxy (such as phenoxy) and R³, R⁴, R⁵, R⁶, n, m, A, B and L have any of the meanings defined hereinbefore except that any functional group is protected if necessary, with an amine of the formula HNR¹R², wherein R¹ and R² have any of the meanings defined hereinbefore except that any functional group is protected if necessary; or
Process (f) The reaction of a compound of the formula XI:

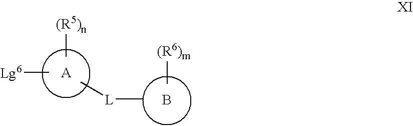

wherein Lg⁶ is a suitable displaceable group for example halogeno (such as chloro, bromo or iodo) or a sulfonyloxy group (such as trifluoromethylsulfonyloxy) and R⁵, R⁶, n, m, A, B and L have any of the meanings defined hereinbefore except that any functional group is protected if necessary, with an alkyne of the formula XII:

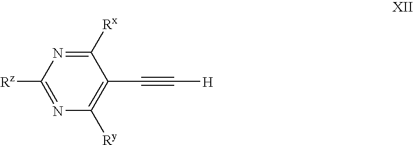

wherein R^{x}, R^{y}, and R^{z} have any of the meanings defined hereinbefore except that any functional group is protected if necessary; or
Process (g) The reaction of a compound of the formula XIII:

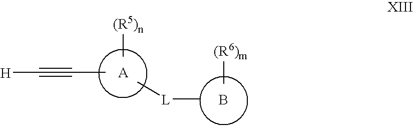

wherein R⁵, R⁶, n, m, A, B and L have any of the meanings defined hereinbefore except that any functional group is protected if necessary, with a pyrimidine of the formula XIV:

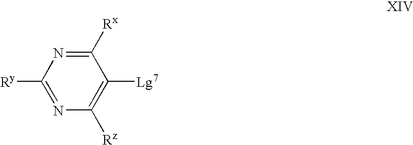

wherein Lg⁷ is a suitable displaceable group for example halogeno (such as chloro, bromo or iodo) or a sulfonyloxy group (such as trifluoromethylsulfonyloxy) and R^{x}, R^{y} and R^{z} have any of the meanings defined hereinbefore except that any functional group is protected if necessary;
or
and thereafter if necessary:
i) converting a compound of the Formula (I) into another compound of the Formula (I);
ii) removing any protecting groups;
iii) forming a salt or solvate.

### Reaction Conditions for Process (a)

When Lg¹ is hydroxy, the reaction of process (a) is conveniently carried out in the presence of a suitable coupling agent. A suitable coupling agent is, for example, a suitable peptide coupling agent, for example O-(7-azabenzotriazol-1-yl)-N,N,N′,N′-tetramethyluronium hexafluorophosphate (HATU) or a suitable carbodiimide such as dicyclohexylcarbodiimide (DCC) or carbonyldiimidazole (CDI), optionally in the presence of a catalyst such as dimethylaminopyridine or hydroxybenzotriazole.

When Lg¹ is any suitable displaceable group as described above, the reaction of process (a) may conveniently be carried out in the presence of a suitable base. A suitable base is, for example, an organic amine base such as, for example, pyridine, 2,6-lutidine, collidine, 4-dimethylaminopyridine, triethylamine, diisopropylethylamine, N-methylmorpholine or diazabicyclo[5.4.0]undec-7-ene. Another suitable base is, for example, an alkali or alkaline earth metal carbonate, for example sodium carbonate, potassium carbonate, caesium carbonate or calcium carbonate.

The reaction of process (a) is conveniently carried out in the presence of a suitable inert solvent or diluent, for example an ester such as ethyl acetate, a halogenated solvent such as dichloromethane, chloroform or carbon tetrachloride, an ether such as tetrahydrofaran or 1,4-dioxane, an aromatic solvent such as toluene or a dipolar aprotic solvent such as N N-dimethylformamide, N,N-dimethylacetamide, N-methylpyrrolidin-2-one or dimethylsulfoxide. The reaction is conveniently carried out at a temperature in the range, for example, from about 0° C. to about 120° C., preferably at or near ambient temperature.

### Reaction Conditions for Process (b)

The reaction of process (b) is conveniently carried out under the conditions as described above for process (a).

### Reaction Conditions for Process (c)

The reaction of process (c) is conveniently carried out in the presence of a suitable base. A suitable base is, for example, an organic amine base such as pyridine or a trialkylamine (such as triethylamine or diisopropylethylamine) or, for example, an alkali or alkaline earth metal carbonate such as sodium carbonate or potassium carbonate.

The reaction of process (c) is conveniently carried out in the presence of a suitable solvent or diluent, for example tetrahydrofuran, 1,4-dioxane or a dipolar aprotic solvent such as dimethylformamide or dimethylacetamide. The reaction is conveniently carried out at a temperature in the range, for example, from about ambient temperature to about 100° C., and under atmospheric pressure.

### Reaction Conditions for Process (d)

The reaction of process (d) is conveniently carried out under the conditions as described above for process (c).

### Reaction Conditions for Process (e)

The reaction of process (e) is conveniently carried out in the presence of a catalytic amount of a suitable acid. A suitable acid is, for example, hydrogen chloride.

The reaction of process (e) may conveniently be carried out in the absence or the presence of a suitable inert solvent or diluent. A suitable inert solvent or diluent, when used, is for example an alcohol such as ethanol, isopropanol or butanol or a dipolar aprotic solvent such as acetonitrile, N N-dimethylformamide, N,N-dimethylacetamide, N-methylpyrrolidin-2-one or dimethylsulfoxide. The reaction is conveniently carried out at a temperature in the range, for example, from ambient temperature to about 120° C., preferably from about 80° C. to about 90° C.

### Reaction Conditions for Process (f)

The reaction of process (f) is conveniently carried out in the presence of a suitable palladium catalyst, optionally in combination with a suitable copper catalyst. A suitable palladium catalyst is, for example, bis(triphenylphosphine)palladium dichloride, [1,1′-bis(diphenylphosphino)ferrocene]palladium dichloride or tetrakis(triphenylphosphine)palladium(0). A suitable copper catalyst is, for example, copper (I) iodide.

The reaction of process (f) is conveniently carried out in the presence of a suitable base. A suitable base is, for example, an organic amine base, such as trialkylamine (for example triethylamine) or tetramethylguanidine.

The reaction of process (f) may conveniently be carried out in the absence or the presence of a suitable inert solvent or diluent, for example an ester such as ethyl acetate, an ether such as tetrahydrofuran or 1,4-dioxane or a dipolar aprotic solvent such as N,N-dimethylformamide, N,N-dimethylacetamide, N-methylpyrrolidin-2-one or dimethylsulfoxide. The reaction is conveniently carried out at a temperature in the range, for example, from about −20° C. to about 100° C.

### Reaction Conditions for Process (g)

The reaction of process (g) is conveniently carried out under the conditions as described above for process (f).

### Starting Materials for Process (a)

Compounds of the formula II may be obtained by conventional procedures. For example, compounds of the formula II may be obtained by reaction of a pyrimidine of the formula XV with an alkyne of the formula XVI as illustrated in Reaction Scheme 1:

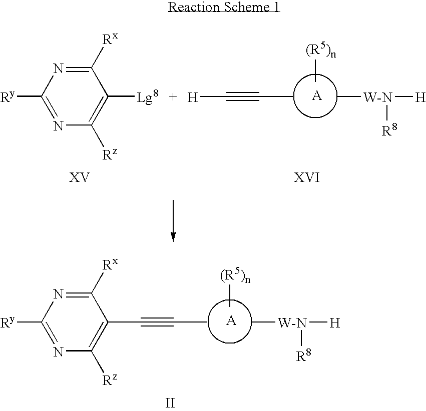

wherein Lg⁸ is a suitable displaceable group as described above and R^{x} R^{y}, R^{z}, R⁵, R⁸, n and A have any of the meanings defined hereinbefore except that any functional group is protected if necessary.

The reaction of Reaction Scheme 1 is conveniently carried out under the conditions as described above for process (f).

Alternatively, compounds of the formula II may be obtained by reaction of a pyrimidine of the formula XVII with a protected alkyne of the formula XVIII and then with an amine of the formula XIX as illustrated in Reaction Scheme 2:

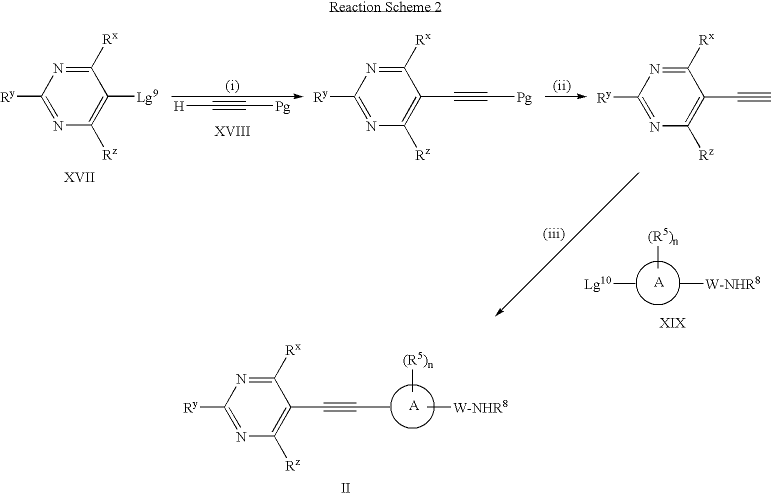

wherein Lg⁹ and Lg¹⁰ are each a suitable displaceable group, Pg is a suitable protecting group, for example a trialkylsilyl group, such as trimethylsilyl or tert-butyldimethylsilyl or Me₂(OH)C— and R^{x}, R^{y}, R^{z}, R⁵, R⁸, n and A have any of the meanings defined hereinbefore except that any functional group is protected if necessary.

Step (i) of Reaction Scheme 2 is the coupling of a protected alkyne of the formula XVIII to a pyrimidine of the formula XVII. Step (i) is carried out under conditions as described above for process (f). Step (ii) of Reaction Scheme 2 is the deprotection of the alkyne under basic or acidic conditions to provide an unprotected alkyne. A person skilled in the art would readily be able to select the appropriate conditions for deprotection in step (ii). Step (iii) of Reaction Scheme 2 is the coupling of the alkyne to an amine of the formula XIX. Step (iii) of Reaction Scheme 2 is carried out under conditions as described above for process (f).

Alternatively, compounds of the formula II where R^{y} is a group NR¹R² may be obtained by reaction of a compound of the formula XX, wherein Lg¹¹ is a suitable displaceable group and R^{3a}, R^{4a}, R⁵, R⁸, n and A have any of the meanings defined hereinbefore except that any functional group is protected if necessary, with an amine of the formula HNR¹R² using reaction conditions as described above for process (e).

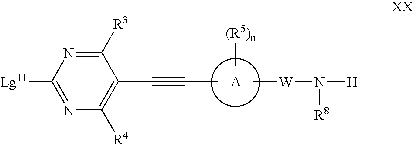

Alternatively, compounds of the formula II where R^{x} is NR¹R² may be obtained by reaction of a compound of the formula XX′, wherein Lg³ is a suitable displaceable group as described above and R^{3b}, R^{4b}, R⁵, R⁸, n and A have any of the meanings defined hereinbefore except that any functional group is protected if necessary, with an amine of the formula HNR¹R² using reaction conditions as described above for process (g).

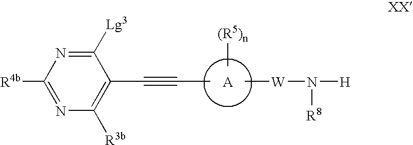

The starting materials of the formulae XV, XVI, XVIII and XIX and the amine HNR¹R² are commercially available or they are known in the literature, or they can be prepared by standard processes known in the art. The starting material of the formula XX can be prepared by standard processes known in the art or by a method analogous to Reaction Scheme 2 as described above.

Compounds of the formula III may be obtained by conventional procedures.

### Starting Materials for Process (b)

Compounds of the formula IV may be obtained by analogous processes to those for the preparation of compounds of Formula II in ‘Starting Materials for Process (a)’ above.

Amines of the formula V are commercially available or they are known in the literature, or they can be prepared by standard processes known in the art.

### Starting Materials for Process (c)

Compounds of the formula VI may be obtained by conventional procedures analogous to the procedures for the preparation of compounds of formula II in ‘Starting Materials for Process (a)’ above.

Compounds of formula VII are commercially available or they are known in the literature, or they can be prepared by standard processes known in the art.

### Starting Materials for Process (d)

Compounds of the formula VIII may be obtained by conventional procedures analogous to the procedures for the preparation of compounds of formula II in ‘Starting Materials for Process (a)’ above.

Compounds of formula XV are commercially available or they are known in the literature, or they can be prepared by standard processes known in the art.

### Starting Materials for Process (e)

As the skilled person would appreciate, compounds of the formula X can be prepared using similar processes to those described above using the appropriate starting materials, for example wherein the starting materials carry an, optionally protected, group Lg³ in place of the —NR¹R² group.

Amines of the formula HNR¹R² are commercially available or they are known in the literature, or they can be prepared by standard processes known in the art.

### Starting Materials for Process (f)

Compounds of formula XI are commercially available or they are known in the literature, or as the skilled person would appreciate they can be prepared using similar processes to those described above using the appropriate starting materials using processes analogous to process a) above.

Alkynes of the formula XII are commercially available or as the skilled person would appreciate they can be prepared using similar processes to those described above using the appropriate starting materials. For example, compounds of the formula XII may conveniently be obtained by reaction of a pyrimidine of the formula XXI:

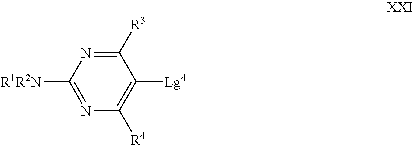

wherein Lg⁴ is a suitable displaceable group and R¹, R², R³ and R⁴ have any of the meanings defined hereinbefore except that any functional group is protected if necessary, with trimethylsilylacetylene or 2-methyl-3-butyn-2-ol conveniently under the conditions as described above in Reaction Scheme (2), followed by the removal of the protecting group using standard procedures known in the art.

### Starting Materials for Process (g)

Compounds of the formula XIII can be prepared using procedures analogous to processes (a)-(b) above.

Compounds of the formula XIVa are commercially available or as the skilled person would appreciate they can be prepared using similar processes to those described above using the appropriate starting materials.

Compounds of the formula I can be converted into further compounds of the formula I using standard procedures conventional in the art.

Examples of the types of conversion reactions that may be used include introduction of a substituent by means of an aromatic substitution reaction or of a nucleophilic substitution reaction, reduction of substituents, alkylation of substituents and oxidation of substituents. The reagents and reaction conditions for such procedures are well known in the chemical art.

Particular examples of aromatic substitution reactions include the introduction of an alkyl group using an alkyl halide and Lewis acid (such as aluminium trichloride) under Friedel Crafts conditions; and the introduction of a halogeno group. Particular examples of nucleophilic substitution reactions include the introduction of an alkoxy group or of a monoalkylamino group, a dialkyamino group or a N-containing heterocycle using standard conditions. Particular examples of reduction reactions include the reduction of a carbonyl group to a hydroxy group with sodium borohydride or of a nitro group to an amino group by catalytic hydrogenation with a nickel catalyst or by treatment with iron in the presence of hydrochloric acid with heating.

An example of a suitable conversion reaction is the conversion of a compound of the formula I wherein R², R³, R⁴, R⁵, R⁶, n, m, A, B and L are as defined in claim 1 and R¹ and/or R² is hydrogen to a compound of the formula I wherein R¹ and/or R² is, for example, an optionally substituted (1-6C)alkoxycarbonyl group. Such a conversion may be achieved using standard procedures, for example by substitution of one or both of the hydrogen atoms R¹ and/or R² for a desired, optionally substituted (1-6C)alkoxycarbonyl group.

Certain compounds of Formula I are capable of existing in stereoisomeric forms. It will be understood that the invention encompasses all geometric and optical isomers of the compounds of formula I and mixtures thereof including racemates. Tautomers and mixtures thereof also form an aspect of the present invention.

Isomers may be resolved or separated by conventional techniques, e.g. chromatography or fractional crystallisation. Enantiomers may be isolated by separation of a racemic or other mixture of the compounds using conventional techniques (e.g. chiral High Performance Liquid Chromatography (HPLC)). Alternatively the desired optical isomers may be made by reaction of the appropriate optically active starting materials under conditions which will not cause racemisation, or by derivatisation, for example with a homochiral acid followed by separation of the diastereomeric derivatives by conventional means (e.g. HPLC, chromatography over silica) or may be made with achiral starting materials and chiral reagents. All stereoisomers are included within the scope of the invention.

The compounds of the invention may be isolated from their reaction mixtures using conventional techniques.

It will be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein. Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Specific examples of protecting groups are given below for the sake of convenience, in which “lower”, as in, for example, lower alkyl, signifies that the group to which it is applied preferably has 1-4 carbon atoms. It will be understood that these examples are not exhaustive. Where specific examples of methods for the removal of protecting groups are given below these are similarly not exhaustive. The use of protecting groups and methods of deprotection not specifically mentioned are, of course, within the scope of the invention.

It will also be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulfinyl or alkylsulfonyl.

It is believed that certain intermediate compounds of Formulae II, IV, VI, VIII, X and XX are novel and are herein claimed as another aspect of the present invention.

### Biological Assays

The following assay can be used to measure the effects of the compounds of the present invention as inhibitors of Tie2 autophosphorylation in whole cells.

### Cellular Tie2 Autophosphorylation Assay

This assay is based on measuring the ability of compounds to inhibit autophosphorylation of the Tie2 receptor which normally leads to the production of “activated” receptor that in turn initiates the particular signal transduction pathways associated with the receptor function.

Autophosphorylation can be achieved by a number of means. It is known that expression of recombinant kinase domains in baculoviral systems can lead to the production of phosphorylated and activated receptor. It is also reported that over expression of receptors in recombinant cell lines can itself lead to receptor autophosphorylation in the absence of the ligand (Heldin C-H. 1995 Cell: 80, 213-223; Blume-J. P, Hunter T. 2001 Nature: 411, 355-65). Furthermore, there are numerous literature examples in which chimaeric receptors have been constructed. In these cases the natural, external cell surface domain of the receptor has been replaced with that of a domain which is known to be readily dimerised via the addition of the appropriate ligand (e.g. TrkA-Tie2/NGF ligand (Marron, M. B., et al., 2000 Journal of Biological Chemistry: 275:39741-39746) or C-fms-Tie-1/CSF-1 ligand (Kontos, C. D., et al., 2002 Molecular and Cellular Biology: 22, 1704-1713). Thus when the chimaeric receptor expressed in a host cell line and the respective ligand is added, this induces autophosphorylation of the chimeric receptor's kinase domain. This approach has the advantage of often allowing a known (and often easily obtained) ligand to be used instead of having to identify and isolate the natural ligand for each receptor of interest.

Naturally if the ligand is available one can use natural cell lines or primary cells which are known to express the receptor of choice and simply stimulate with ligand to achieve ligand induced phosphorylation. The ability of compounds to inhibit autophosphorylation of the Tie2 receptor, which is expressed for example in EA.hy926/B3 cells (supplied by J. McLean/B. Tuchi, Univ. of N. Carolina at Chapel Hill, CB-4100, 300 Bynum Hall, Chapel Hill, N.C. 27599-41000, USA) or primary HUVEC (human umbilical vein endothelial cells—available from various commercial sources), can measured by this assay.

Natural Ang1 ligand can be isolated using standard purification technology from either tumour cell supernatants or alternatively the Angl gene can be cloned and expressed recombinantly using stand molecular biology techniques and expression systems. In this case one can either attempt to produce the ligand either in its native state or as recombinant protein which for example may have been genetically engineered to contain additional of purification tags (e.g. polyhistidine peptides, antibody Fc domains) to facilitate the process.

Using the ligand stimulation of either EA.hy926/B3 or HUVEC cellular Tie2 receptor as the example, a Ang1 ligand stimulated cellular receptor phosphorylation assay can be constructed which can be used to analyse to determine the potential of compounds to inhibit this process. For example EA.hy926/B3 cells were grown in the appropriate tissue culture media plus 10% foetal calf serum (FCS) for two days in 6 well plates starting with an initial seeding density of 5×10⁵ cells/well. On the third day the cells were serum starved for a total of 2 hours by replacing the previous media with media containing only 1% FCS. After 1 hour 40 minutes of serum starvation the media was removed and replace with 1 ml of the test compound dilutions (compound dilutions made in serum starvation media yet keeping the DMSO concentration below 0.8%). After 1.5 hours of serum starvation orthovanidate was added to a final concentration of 0.1 mM for the final 10 minutes of serum starvation.

Following a total of 2 hours of serum starvation, the ligand plus orthovandiate was added to stimulate autophosphorylation of the cellular Tie2 receptor (ligand can be added either as purified material diluted in serum starvation media or non-purified cell supernatant containing ligand e.g. when recombinantly expressed mammalian cells).

After 10 minutes incubation at 37° C. with the ligand, the cells were cooled on ice washed with approximately 5 m/s with cold PBS containing 1 mM orthovanadate, after which 1 ml of ice cold lysis buffer ((20 mM Tris pH 7.6, 150 mM NaCl, 50 mM NaF, 0.1% SDS, 1% NP40, 0.5 % DOC, 1 mM orthovanadate, 1 mM EDTA, 1 mM PMSF, 30 μl/ml Aprotinin, 10 μg/ml Pepstatin, 10 μg/ml Leupeptin) was added the cells and left on ice for 10-20 minutes. The lysate was removed and transferred to a 1.5 ml Eppendorf tube and centrifuged for 3 minutes at 13000 rpm at 4° C. 800 μl of each lysate was transferred to fresh 2 ml Eppendorf tubes for the immuno-precipitation. 3 mg=15 μl of anti-phospho-tyrosine antibody (Santa Cruz PY99—sc-7020) was added to the lysates and left to incubate for 2 hours at 4° C. 600 μl washed MagnaBind beads (goat anti-mouse IgG, Pierce 21354) were added to the lysates and the tubes left to rotate over night at 4° C.

Samples were treated for 1 minute in the magnet before carefully removing the lysis supernatant. 1 ml of lysis buffer was then added to the beads and this step repeated twice more. The beads were suspended in 25 μl of 94° C. hot 2 □ Laemmli loading buffer plus beta-mercaptoethanol and left to stand for 15 minutes at room temperature.

The beads were removed by exposing the tubes for 1 minutes in the magnet, and the total liquid separated from the beads from each immuno-precipitate loaded onto Polyacrylamide/SDS protein gels (pre-cast 4-12% BisTris NuPAGE/MOPS 12 well gels from Novex). Protein gels were run at 200 V and then blotted onto NC membrane for 1 hours 30 minutes at 50 V/250 mA. All blots were treated with 5% Marvel in PBS-Tween for 1 hour at room temperature to reduce non-specific binding of the detection antibody. A rabbit anti-Tie2 (Santa Cruz sc-324) was added in a 1:500 dilution in 0.5 % Marvel/PBS-Tween and left to incubate overnight at 4° C. The blots were rigorously washed with PBS-Tween before adding the goat anti rabbit -POD conjugate (Dako P0448) at a 1:5000 dilution in 0.5% Marvel/PBS-Tween. The antibody was left on for 1 hour at room temperature before subsequently washing the blots with PBS-Tween. The western blots of the various immuno-precipitated samples were developed the blots with LumiGLO (NEB 7003). And transferred to an X-Ray cassette and films exposed for 15 sec/30 sec and 60 sec. The relative strength of the protein band which pertains to the phosphorylated Tie2 receptor was evaluated using a FluorS BioRad image analyser system. The percentage phosphorylation for each test compound dilution series was determined from which IC₅₀ values were calculated by standard methods using the appropriate control samples as reference.

Although the pharmacological properties of the compounds of the Formula I vary with structural change as expected, in general activity possessed by compounds of the Formula I, is in the range of IC₅₀ of <50 μM in the above test:-

By way of example, Table A illustrates the activity of representative compounds according to the invention showing IC₅₀ data for the inhibition of autophosphorylation of Tie2 receptor tyrosine kinase.

TABLE A

| | IC₅₀ (□M) |
|---|---|
| | Inhibition of |
| | autophosphorylation |
| | of Tie2 receptor |
| Example Number | tyrosine kinase |
| 1 | 0.016 |
| 2 | 0.026 |

In the following section references to a compound of formula I, refer also to other sub-groups of the invention as described above, for example would also apply, amongst other sub-groups of the invention, to compounds of formula Ia, Ib, Ic and Id.

According to a further aspect of the invention there is provided a pharmaceutical composition which comprises a compound of the Formula I, or a pharmaceutically acceptable salt thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the Formula I will naturally vary according to the nature and severity of the conditions, the age and sex of the animal or patient and the route of administration, according to well known principles of medicine.

In using a compound of the Formula I for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration is however preferred, particularly in tablet form. Typically, unit dosage forms will contain about 0.5 mg to 0.5 g of a compound of this invention.

The compounds according to the present invention as defined herein are of interest for, amongst other things, their antiangiogenic effect. The compounds of the invention are expected to be useful in the treatment or prophylaxis of a wide range of disease states associated with undesirable or pathological angiogenesis, including cancer, diabetes, psoriasis, rheumatoid arthritis, Kaposi's sarcoma, haemangioma, lymphoedema, acute and chronic nephropathies, atheroma, arterial restenosis, autoimmune diseases, acute inflammation, excessive scar formation and adhesions, endometriosis, dysfunctional uterine bleeding and ocular diseases with retinal vessel proliferation. Cancer may affect any tissue and includes leukaemia, multiple myeloma and lymphoma. In particular such compounds of the invention are expected to slow advantageously the growth of primary and recurrent solid tumours of, for example, the colon, breast, prostate, lungs and skin.

We believe that the antiangiogenic properties of the compounds according to the present invention arise from their Tie2 receptor tyrosine kinase inhibitory properties. Accordingly, the compounds of the present invention are expected be useful to produce a Tie2 inhibitory effect in a warm-blooded animal in need of such treatment. Thus the compounds of the present invention may be used to produce an antiangiogenic effect mediated alone or in part by the inhibition of Tie2 receptor tyrosine kinase.

More particularly the compounds of the invention are expected to inhibit any form of cancer associated with Tie2. For example, the growth of those primary and recurrent solid tumours which are associated with Tie2, especially those tumours which are significantly dependent on Tie2 receptor tyrosine kinase for their growth and spread.

According to a further aspect of the invention there is provided a compound of the Formula I, or a pharmaceutically-acceptable salt thereof, as defined hereinbefore, for use as a medicament.

According to another aspect of the invention, there is provided the use of a compound of the formula I, or a pharmaceutically acceptable salt thereof, as defined hereinbefore, in the manufacture of a medicament for use as a Tie2 receptor tyrosine kinase inhibitor in a warm-blooded animal such as man.

According to another aspect of the invention, there is provided the use of a compound of the formula I, or a pharmaceutically acceptable salt thereof, as defined hereinbefore, in the manufacture of a medicament for use in the production of an anti-angiogenic effect in a warm-blooded animal such as man.

According to another aspect of the invention, there is provided the use of a compound of the formula I, or a pharmaceutically acceptable salt thereof, as defined hereinbefore in the manufacture of a medicament for use in the treatment of cancers in a warm-blooded animal such as man.

According to another aspect of the invention, there is provided the use of a compound of the formula I, or a pharmaceutically acceptable salt thereof, as defined hereinbefore in the manufacture of a medicament for use in the treatment of a cancer selected from leukaemia, breast, lung, colon, rectal, stomach, prostate, bladder, pancreas, ovarian, lymphoma, testicular, neuroblastoma, hepatic, bile duct, renal cell, uterine, thyroid and skin cancer in a warm-blooded animal such as man.

According to another aspect of the invention there is provided a method of inhibiting Tie2 receptor tyrosine kinase in a warm-blooded animal, such as man, in need of such treatment, which comprises administering to said animal an effective amount of a compound of the formula I, or a pharmaceutically acceptable salt thereof, as defined hereinbefore.

According to another aspect of the invention there is provided a method for producing an anti-angiogenic effect in a warm-blooded animal, such as man, in need of such treatment, which comprises administering to said animal an effective amount of a compound of the formula I, or a pharmaceutically acceptable salt thereof, as defined hereinbefore.

According to another aspect of the invention there is provided a method of treating cancers in a warm-blooded animal, such as man, in need of such treatment, which comprises administering to said animal an effective amount of a compound of the formula I, or a pharmaceutically acceptable salt thereof, as defined hereinbefore.

According to another aspect of the invention there is provided a method of treating a cancer selected from leukaemia, breast, lung, colon, rectal, stomach, prostate, bladder, pancreas, ovarian, lymphoma, testicular, neuroblastoma, hepatic, bile duct, renal cell, uterine, thyroid or skin cancer, in a warm-blooded animal, such as man, in need of such treatment, which comprises administering to said animal an effective amount of a compound of the formula I, or a pharmaceutically acceptable salt thereof, as defined hereinbefore.

According to another aspect of the invention there is provided a compound of the formula I, or a pharmaceutically acceptable salt thereof, as defined hereinbefore, for use in inhibiting Tie2 receptor tyrosine kinase in a warm-blooded animal, such as man.

According to an another aspect of the invention there is provided a compound of the formula I, or a pharmaceutically acceptable salt thereof, as defined hereinbefore, for use in producing an anti-angiogenic effect in a warm-blooded animal, such as man.

According to another aspect of the invention there is provided a compound of the formula I, or a pharmaceutically acceptable salt thereof, as defined hereinbefore, for use in the treatment of cancer.

According to another aspect of the invention there is provided a compound of the formula I, or a pharmaceutically acceptable salt thereof, as defined hereinbefore, for use in the treatment of a cancer selected from leukaemia, breast, lung, colon, rectal, stomach, prostate, bladder, pancreas, ovarian, lymphoma, testicular, neuroblastoma, hepatic, bile duct, renal cell, uterine, thyroid or skin cancer.

As hereinbefore mentioned it is further expected that a compound of the present invention will possess activity against other diseases mediated by undesirable or pathological angiogenesis including psoriasis, rheumatoid arthritis, Kaposi's sarcoma, haemangioma, lymphoedema, acute and chronic nephropathies, atheroma, arterial restenosis, autoimmune diseases, acute inflammation, excessive scar formation and adhesions, endometriosis, dysfunctional uterine bleeding and ocular diseases with retinal vessel proliferation.

The anti-angiogenic activity defined herein may be applied as a sole therapy or may involve, in addition to a compound of the invention, one or more other substances and/or treatments. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the cell cycle inhibitory treatment defined hereinbefore may be: surgery, radiotherapy or chemotherapy. Such chemotherapy may include one or more of the following categories of anti-tumour agents:
(i) anti-invasion agents (for example metalloproteinase inhibitors like marimastat and inhibitors of urokinase plasminogen activator receptor function);
(ii) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea, or, for example, one of the preferred antimetabolites disclosed in European Patent Application No. 562734 such as
(2S)-2-{o-fluoro-p-[N-{2,7-dimethyl-4-oxo-3,4-dihydroquinazolin-6-ylmethyl)-N-(prop-2-ynyl)amino]benzamido}-4-(tetrazol-5-yl)butyric acid); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(iii) cytostatic agents such as antioestrogens (for example tamoxifen, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrazole, vorazole and exemestane) and inhibitors of 5a-reductase such as finasteride;
(iv) inhibitors of growth factor function, for example such inhibitors include growth factor antibodies, growth factor receptor antibodies, famesyl transferase inhibitors, tyrosine kinase inhibitors and serine/threonine kinase inhibitors, for example inhibitors of the epidermal growth factor family (for example the EGFR tyrosine kinase inhibitors N-(3-chloro-4-fluorophenyl)-7-methoxy-6-(3-morpholinopropoxy)quinazolin-4-amine (ZD1839), N-(3-ethynylphenyl)-6,7-bis(2-methoxyethoxy)quinazolin-4-amine (CP 358774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents that work by different mechanisms to those defined hereinbefore, such as those which inhibit vascular endothelial growth factor such as the compounds disclosed in International Patent Applications WO 97/22596, WO 97/30035, WO 97/32856 and WO 98/13354 and those that work by other mechanisms (for example linomide, inhibitors of integrin αvβ3 function and angiostatin);
(vi) biotherapeutic therapeutic approaches for example those which use peptides or proteins (such as antibodies or soluble external receptor domain constructions) which either sequest receptor ligands, block ligand binding to receptor or decrease receptor signalling (e.g. due to enhanced receptor degradation or lowered expression levels)
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a pharmaceutical product comprising a compound of the Formula I as defined hereinbefore and an additional anti-tumour substance as defined hereinbefore for the conjoint treatment of cancer.

In addition to their use in therapeutic medicine, the compounds of Formula I and their pharmaceutically acceptable salts, are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

Examples of compounds of the invention include:
5-[(4-Aminopyrimidin-5-yl)ethynyl]-N-[2-fluoro-5-(trifluoromethyl)phenyl]thiophene-2-carboxamide;
4-[(4-Aminoprimidin-5-yl)ethynyl]-N-[2-fluoro-5-(trifluoromethyl)phenyl]thiophene-2-carboxamide;
5-[(4-Aminopyrimidin-5-yl)ethynyl]-N-(5-tert-butylisoxazol-3-yl)thiophene-2-carboxamide;
2-[(4-Aminopyrimidin-5-yl)ethynyl]-N-phenyl-1,3-thiazole-4-carboxamide;
2-[(4-Aminopyrimidin-5-yl)ethynyl]-N-(2,2-dimethyltetahydro-2H-pyran-4-yl)-1,3-thiazole-4-carboxamide;
2-[(4-Aminopyrimidin-5-yl)ethynyl]-N-(5-tert-butylisoxazol-3-yl)-1,3-thiazole-4-carboxamide;
and salts thereof, as well as the compounds listed in the following examples.

The invention will now be illustrated by the following non-limiting examples in which, unless stated otherwise:
(i) temperatures are given in degrees Celsius (° C.); operations were carried out at room or ambient temperature, that is, at a temperature in the range of 18-25° C.;
(ii) organic solutions were dried over anhydrous magnesium sulfate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and/or analytical LC-MS, and reaction times are given for illustration only;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 300 MHz using perdeuterio dimethyl sulphoxide (DMSO-d₆) as solvent unless otherwise indicated; the following abbreviations have been used: s, singlet; d, doublet; t, triplet; q, quartet; m, multiplet; b, broad;
(viii) chemical symbols have their usual meanings; SI units and symbols are used;
(ix) solvent ratios are given in volume:volume (v/v) terms; and
(x) mass spectra (MS) were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is MH⁺;
(xi) unless stated otherwise compounds containing an asymmetrically substituted carbon and/or sulphur atom have not been resolved;
(xii) where a synthesis is described as being analogous to that described in a previous example the amounts used are the millimolar ratio equivalents to those used in the previous example;
(xvi) the following abbreviations have been used:
AcOH Acetic acid
AIBN 2,2′-Azobisisobutyronitrile
DCM Dichloromethane
DIPEA Diisopropylethylamine
DMA N,N-Dimethylacetamide
DMF N,N-Dimethylformamide
DMSO Dimethylsulfoxide
DMTMM 4-(4,6-Dimethoxy-1,3,5-triazin-2-yl)-4-methylmorpholin-4-ium chloride
dppf 1,1′-Bis(diphenylphosphino)ferrocene
EtOAc Ethylacetate
HATU O-(7-Azabenzotriazol-1-yl)-N,N,N′,N′-tetramethyluronium hexafluorophosphate
ⁱPrMgCl Isopropylmagnesium chloride
LDA Lithium diisopropylamide
LHMDS Lithium bis(trimethylsilyl) amide
m-CPBA meta-Chloroperbenzoic acid
MeOH Methanol
MeCN Acetonitrile
MCX Mixed cation exchange resin
MTBE Methyl tert-butyl ether
LCMS Liquid Chromatograpy—Mass Spectrometry
NMP 1-Methyl-2-pyrrolidinone
POCl₃ Phosphorus oxychloride
RPHPLC Reversed phase high peformance liquid chromatography
TFA Trifluoroacetic acid
THF Tetrahydrofaran
xvii) where a synthesis is described as leading to an acid addition salt (e.g. HCl salt), no comment is made on the stoichiometry of this salt. Unless otherwise stated, all NMR data is reported on free-base material, with isolated salts converted to the free-base form prior to characterisation.

### EXAMPLE 1

### 5-[(2-Aminopyrimidin-5-yl)ethynyl]-N-[2-fluoro-5-(trifluoromethyl)phenyl]thiophene-2-carboxamide

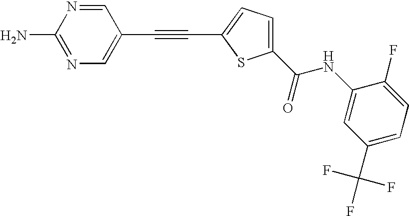

A mixture of 2-amino-5-ethynylpyrimidine (Intermediate 2) (119 mg), 5-bromo-N-[2-fluoro-5-(trifluoromethyl)phenyl]thiophene-2-carboxamide (Intermediate 3) (368 mg), copper (I) iodide (5 mg) and tetrakis(triphenylphosphine)palladium(0) (58 mg) in dry DMF (1.5 mL) was stirred and degassed with nitrogen. 1,1,3,3-Tetramethylguanidine (138 mg) in DMF (0.5 mL) was added and the mixture heated at 60° C. for 3 hours. The mixture was cooled, stirred, and diluted with water (20 mL). The solid formed was filtered off and dried at 60° C. in vacuo. Purification by flash chromatography on silica using 0-50% MeOH in DCM as eluent, then trituration with DCM gave the title compound as a solid (212 mg, 52%);

¹H NMR (DMSO-d₆) 7.23 (s, 2H), 7.42 (d, 1H), 7.55 (m, 1H), 7.67 (m, 1H), 7.99 (d, 1H), 8.04 (m, 1H), 8.45 (s, 2H), 10.48 (bs, 1H);

MS m/e MH⁺+MeCN 448.

### Intermediate 1

### 5-[(Trimethylsilyl)ethynyl]pyrimidin-2-amine

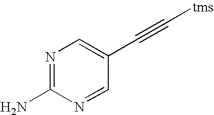

PdCl₂dppf (146 mg) was added to a solution of 2-amino-5-iodopyrimidine (221 mg), trimethylsilylacetylene (491 mg), CuI (57 mg) and DIPEA (259 mg) in EtOAc (5 mL) at −20° C. under an inert atmosphere. The reaction was allowed to warm to ambient temperature and stirred for 6 hours. The reaction mixture was diluted with water (10 mL). The organic layer was separated, dried (MgSO₄), filtered and concentrated. The crude product was used directly without further purification (191 mg, 100%);

¹H NMR (CDCl₃) 0.26 (s, 9H), 5.19 (bs, 2H), 8.39 (s, 2H);

MS m/e MH⁺+MeCN 233.

### Intermediate 2

### 5-Ethynylpyrimidin-2-amine

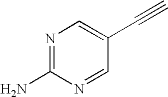

Potassium carbonate (276 mg) was added to a solution of 5-[(trimethylsilyl)ethynyl]pyrimidin-2-amine (Intermediate 1) (191 mg) in MeOH (40 mL)/water (20 mL). The reaction mixture was stirred at ambient temperature under an inert atmosphere for 24 hours then neutralised with 1M HCl. The reaction mixture was then concentrated and the resultant residue dissolved in DCM (30 mL). The DCM phase was washed with water (15 mL), brine (15 mL), dried (MgSO₄), filtered and concentrated. The crude product was used directly without further purification (119 mg, 100%);

¹H NMR (CDCl₃) 3.19 (s, 1H), 5.26 (bs, 2H), 8.41 (s, 2H);

MS m/e MH⁺+MeCN 161.

### Intermediate 3

### 5-Bromo-N-[2-fluoro-5-(trifluoromethyl)phenyl]thiophene-2-carboxamide

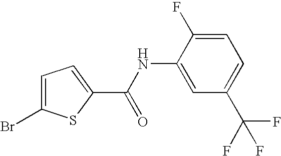

5-Bromothiophene-2-carbonyl chloride (1.12 g) in dry DCM (5 mL) was added dropwise to a solution of 2-fluoro-5-(trifluoromethyl)aniline (0.90 g) and triethylamine (3 mL) in DCM and stirred for 60 hours at ambient temperature. The solvent was removed by evaporation and the residue was stirred with 40% aqueous sodium hydroxide (1.0 mL) in MeOH (10 mL) for 17 hours. The mixture was concentrated then partitioned between DCM and water. The organic phase was washed with 2M hydrochloric acid then aqueous sodium bicarbonate, dried, and filtered through a short silica column. Concentration, then crystallization from isohexane gave the product (0.51 g, 27%);

¹H NMR (DMSO-d₆) 7.39 (d, 1H), 7.58 (m, 1H), 7.69 (m, 1H), 7.89 (d, 1H), 8.05 (m, 1H), 10.45 (s, 1H);

MS m/e MH⁺+CH₃CN 411, 409 (1×Br).

### EXAMPLE 2

### 4-[(2-Aminopyrimidin-5-yl)ethynyl]-N-[2-fluoro-5-(trifluoromethyl)phenyl]thiophene-2-carboxamide

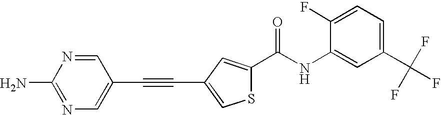

The title compound was prepared in a similar manner to Example 1.

SM: 2-amino-5-ethynylpyrimidine (Intermediate 2), 4-bromo-N-[2-fluoro-5-(trifluoromethyl)phenyl]thiophene-2-carboxamide (Intermediate 4)

¹H NMR (DMSO-d₆) 7.13 (s, 2H), 7.57 (m, 1H), 7.67 (m, 1H), 8.06 (m, 1H), 8.12 (d, 2H), 8.41 (s, 2H), 10.41 (bs, 1H);

MS m/e MH⁺+CH₃CN 448.

### Intermediate 4

### 4-bromo-N-[2-fluoro-5-(trifluoromethyl)phenyl]thiophene-2-carboxamide

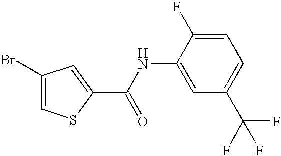

The title compound was prepared in a similar manner to Intermediate 3 except the product was isolated by trituration with isohexane.

SM: 4-bromothiophene-2-carbonyl chloride, 2-fluoro-5-(trifluoromethyl)aniline.

¹H NMR (DMSO-d₆) 7.58 (m, 1H), 7.68 (m, 1H), 8.08 (m, 3H), 10.47 (s, 1H);

MS m/e MH⁺+CH₃CN 411, 409 (1×Br).

### EXAMPLE 3

### 5-[(2-Aminopyrimidin-5-yl)ethynyl]-N-(5-tert-butylisoxazol-3-yl)thiophene-2-carboxamide

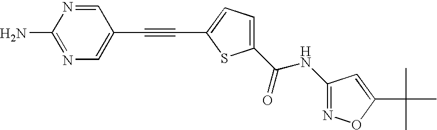

The title compound was prepared in a similar manner to Example 1.

SM: 2-amino-5-ethynylpyrimidine (Intermediate 2), 5-bromo-N-(5-tert-butylisoxazol-3-yl)thiophene-2-carboxamide (Intermediate 5)

¹H NMR (DMSO-d₆) 1.30 (s, 9H), 6.67 (s, 1H), 7.23 (s, 2H), 7.39 (d, 1H), 8.09 (d, 1H), 8.44 (s, 2H), 11.52 (bs, 1H);

MS m/e (M−H)⁻366.

### Intermediate 5

### 5-Bromo-N-(5-tert-butylisoxazol-3-yl)thiophene-2-carboxamide

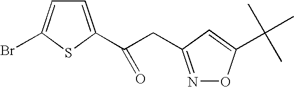

The title compound was prepared in a similar manner to Intermediate 3.

SM: 5-bromothiophene-2-carbonyl chloride, 5-tert-butylisoxazol-3-amine

¹H NMR (DMSO-d₆) 1.32 (s, 9H), 6.67 (s, 1H), 7.37 (d, 1H), 7.99 (d, 1H), 11.51 (s, 1H);

MS m/e MH⁺331, 329 (1×Br).

### EXAMPLE 4

### 2-[(2-Aminopyrimidin-5-yl)ethynyl]-N-phenyl-1,3-thiazole-4-carboxamide

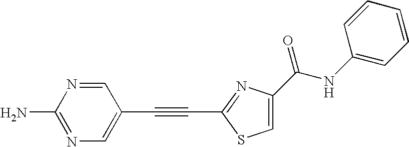

Triethylamine (300 mg) was added to a mixture of 2-[(2-aminopyrimidin-5-yl)ethynyl]-1,3-thiazole-4-carboxylic acid (Intermediate 7) (197 mg) and O-benzotriazol-1-yl-N,N,N′,N′-tetramethyluronium hexafluorophosphate (606 mg) in DMF (5 mL) and stirred for 10 min. Aniline (149 mg) was added and stirred for 60 hours at ambient temperature. The mixture was concentrated then partitioned between EtOAc and aqueous sodium bicarbonate. The solid formed was filtered off, the EtOAc extract was washed with water, dried and concentrated in vacuo. The combined solids were triturated with 1:1 DCM-MeOH to give the title compound (126 mg, 46%);

¹H NMR (DMSO-d₆) 7.11 (m, 1H), 7.38 (m, 4H), 7.85 (m, 2H), 8.55 (m, 3H), 10.35 (s, 1H);

MS m/e MH⁺322.

### Intermediate 6

### Ethyl 2-[(2-aminopyrimidin-5-yl)ethynyl]-1,3-thiazole-4-carboxylate

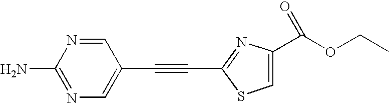

Triethylamine (10 mL) was added to a stirred mixture of 2-amino-5-ethynylpyrimidine (Intermediate 2) (1.19 g), ethyl 2-bromo-1,3-thiazole-4-carboxylate (2.36 g), dichlorobis(triphenylphosphine)palladium(II) (140 mg), and copper (I) iodide (70 mg) in dry THF (100 mL). The mixture was degassed with nitrogen and heated at 60° C. for 3 hours. The mixture was cooled, diluted with water, and concentrated. The solid formed was filtered off, washed with water then dried at 60° C. in vacuo. Purification by flash chromatography on silica using 0-10% MeOH in DCM as eluent, then trituration with DCM/isohexane gave the title compound as a solid (1.5 g, 54%);

¹H NMR (DMSO-d₆) 1.30 (t, 3H), 4.31 (q, 2H), 7.37 (s, 2H), 8.52 (s, 2H), 8.58 (s, 1H);

MS m/e MH⁺275.

### Intermediate 7

### 2-[(2-Aminopyrimidin-5-yl)ethynyl]-1,3-thiazole-4-carboxylic acid

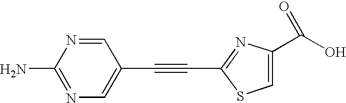

Ethyl 2-[(2-aminopyrimidin-5-yl)ethynyl]-1,3-thiazole-4-carboxylate (Intermediate 6) (1.2 g) was added to a stirred solution of sodium hydroxide (0.8 g) in water (40 mL) and heated at 70° C. for 3 hours. The mixture was acidified with glacial AcOH, stirred and cooled. The solid formed was filtered off, washed with water and dried at 60° C. in vacuo to give the product (1.0 g, 92%);

¹H NMR (DMSO-d₆) 7.37 (s, 2H), 8.50 (m, 3H);

MS m/e MH⁺247.

### EXAMPLE 5

### 2-[(2-Aminopyrimidin-5-yl)ethynyl]-N-(2,2-dimethyltetrahydro-2H-pyran-4-yl)-1,3-thiazole-4-carboxamide

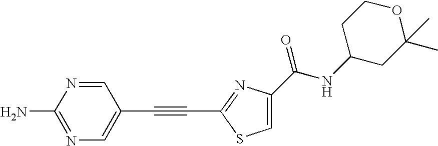

The title compound was prepared in a similar manner to Example 4.

SM: 2-[(2-aminopyrimidin-5-yl)ethynyl]-1,3-thiazole-4-carboxylic acid (Intermediate 7), 4-amino-2,2-dimethyltetrahydropyran

¹H NMR (DMSO-d₆) 1.14 (s, 3H), 1.20 (s, 3H), 1.43-1.72 (m, 4H), 3.65 (m, 2H), 4.18 (m, 1H), 7.39 (s, 2H), 8.30 (d, 1H), 8.36 (s, 2H).

### EXAMPLE 6

### 2-[(2-Aminopyrimidin-5-yl)ethynyl]-N-(5-tert-butylisoxazol-3-yl)-1,3-thiazole-4-carboxamide

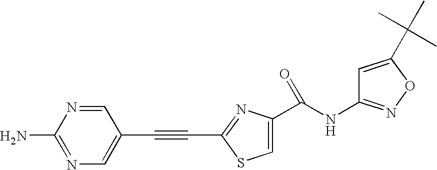

The title compound was prepared in a similar manner to Example 4.

SM: 2-[(2-aminopyrimidin-5-yl)ethynyl]-1,3-thiazole-4-carboxylic acid (Intermediate 7), 5-tert-butylisoxazol-3-amine.

¹H NMR (DMSO-d₆) 1.33 (s, 9H), 6.66 (s, 1H), 7.40 (s, 2H), 8.55 (s, 2H), 8.61 (s, 1H), 11.02 (s, 1H);

MS m/e MH⁺369.

### EXAMPLE 7

### N-{5-[(2-aminopyrimidin-5-yl)ethynyl]pyridin-3-yl}-3-tert-butyl-1-methyl-1H-pyrazole-5-carboxamide

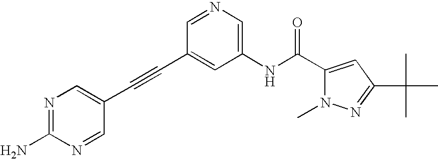

DIPEA (387 mg) was added to a mixture of 5-[(5-aminopyridin-3-yl)ethynyl]pyrimidin-2-amine (Intermediate 11) (211 mg), 3-tert-butyl-1-methyl-1H-pyrazole-5-carboxylic acid (364 mg) and HBTU (642 mg) in DMF (3 mL) and stirred for 65 hours at ambient temperature. The mixture was added dropwise to stirred 1M aqueous sodium hydroxide (100 mL). The solid formed was filtered off, and purified by RPHPLC to give the title compound (121 mg, 32%);

¹H NMR (DMSO-d₆) 1.28 (s, 9H), 4.02 (s, 3H), 6.97 (s, 1H), 7.19 (s, 2H) 8.32 (t, 1H), 8.42 (d, 1H), 8.47 (s, 2H), 8.79 (d, 1H) 10.39 (br s, 1H);

MS m/e MH⁺376.

### Intermediate 8

### tert-butyl (5-bromopyridin-3-yl)carbamate

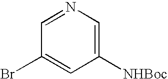

Triethylamine (7 mL) followed by DPPA (10.9 mL) was added to a solution of 5-bromonicotinic acid (10.1 g) and t-BuOH (7.1 mL) in toluene (100 mL) and the reaction heated at reflux under an inert atmosphere for 1.5 hours. The reaction mixture was diluted with EtOAc (100 mL) and water (100 mL). The organic layer was separated, washed with NaHCO₃ (3×50 mL), dried (MgSO₄), filtered and concentrated in vacuo. The product was purified by flash chromatography on silica using 0-4% EtOAc in DCM as the eluent to give the title compound as a beige solid (9.82 g, 72%);

¹H NMR (DMSO-d₆) 1.50 (s, 9H), 8.19 (t, 1H), 8.30 (d, 1H), 8.57 (d, 1H), 9.80 (s, 1H);

MS m/e MH⁺273/275.

### Intermediate 9

### tert-Butyl (5-iodopyridin-3-yl)carbamate

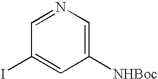

tert-Butyl (5-bromopyridin-3-yl)carbamate (Intermediate 8) (14.9 g), CuI (520 mg), NaI (16.35 g) and N,N-dimethylethylenediamine (481 mg) in dioxane (300 mL) were heated at 110° C. under an inert atmosphere for 24 hours. The reaction mixture was concentrated in vacuo to approx 100 mL and then water (400 mL) was added. The resultant solid was filtered, dissolved in DCM, dried (MgSO4), filtered and concentrated to afford the title compound as a beige solid (15.18 g, 87%);

MS m/e MH⁺321.

### Intermediate 10

### tert-butyl {5-[(2-aminopyrimidin-5-yl)ethynyl]pyridin-3-yl}carbamate

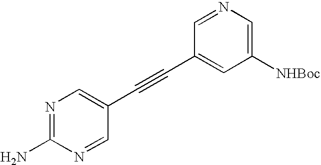

PdCl₂dppf (907 mg) was added to a degassed solution of tert-butyl (5-iodopyridin-3-yl)carbamate (Intermediate 9) (7.94 g), 5-ethynylpyrimidin-2-amine (Intermediate 2) (3.7 g), CuI (94 mg) and Et₃N (63 mL) in DMF (250 mL). The reaction was allowed to stir at ambient temperature under an inert atmosphere for 24 hours. Silica was added to the reaction mixture and then solvent was evaporated in vacuo. The preabsorbed product was purified by flash chromatography on silica using 0-10% MeOH in DCM as the eluent followed by an aqueous wash and then dried in vacuo to give the title compound as a beige solid (5.3 g, 69%);

¹H NMR (DMSO-d₆) 1.51 (s, 9H), 7.20 (s, 2H), 8.05 (s, 1H), 8.31 (s, 1H), 8.48 (s, 2H), 8.57 (s, 1H), 9.74 (s, 1H);

MS m/e (M−H⁺)⁻310.

### Intermediate 11

### 5-[(5-aminopyridin-3-yl)ethynyl]pyrimidin-2-amine

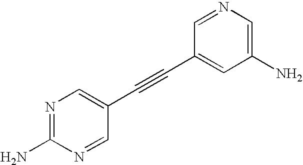

TFA (25 mL) was added to a solution of tert-butyl {5-[(2-aminopyrimidin-5-yl)ethynyl]pyridin-3-yl}carbamate (Intermediate 10) (2.92 g) in DCM (150 mL) under an inert atmosphere. The reaction was allowed to stir at ambient temperature for 3 hours. It was then diluted with water (100 mL) and the DCM removed in vacuo. The aqueous solution was neutralised with NaHCO₃ and the resultant precipitate was filtered, washed with water and then dried in vacuo to give the title compound (2.00 g, 66%);

¹H NMR (DMSO-d₆) 5.49 (s, 2H), 7.00 (s, 1H), 7.88 (s, 2H), 8.44 (s, 2H);

MS m/e MH⁺212.

The following Example was prepared in a similar manner to Example 7 by using Intermediate 11 with the appropriate acid.

### EXAMPLE 8

### N-{5-[(2-aminopyrimidin-5-yl)ethynyl]pyridin-3-yl}-3-(1-cyanoethyl)benzamide

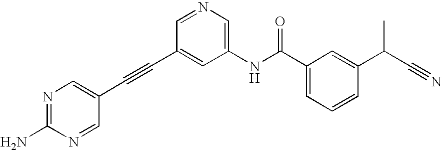

SM: 5-[(5-aminopyridin-3-yl)ethynyl]pyrimidin-2-amine (Intermediate 11) and 3-(1-cyanoethyl)benzoic acid.

¹H NMR (DMSO-d₆) 1.60 (d, 3H), 4.42 (q, 1H), 7.19 (s, 2H) 7.63 (m, 2H), 7.96 (m 2H), 8.35 (t, 1H), 8.44 (d, 1H), 8.47 (s, 2H), 8.87 (d, 1H) 10.59 (br s, 1H);

MS m/e MH⁺369.

### EXAMPLE 9

### 3-morpholin-4-yl-N-[3-({2-[(3-morpholin-4-ylpropyl)amino]pyrimidin-5-yl}ethynyl)phenyl]benzamide

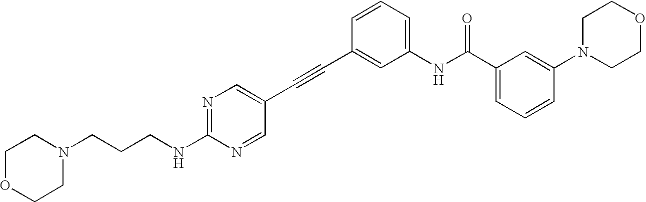

5-[(3-aminophenyl)ethynyl]-N-(3-morpholin-4-ylpropyl)pyrimidin-2-amine (Intermediate 13) (337 mg), 3-morpholin-4-ylbenzoic acid (248 mg) and HBTU (455 mg) were stirred in DMF (5 mL). DIPEA (0.42 mL) was added and the resultant mixture stirred for 16 hours. The reaction was poured into water (20 mL) and extracted with DCM (20 mL). The organics were evaporated and the residue purified by reverse phase HPLC to afford the title compound as a pale yellow solid (126 mg, 24%).

¹H NMR (DMSO-d₆) 10.2 (s, 1H), 8.46 (s, 2H), 7.97 (s, 1H), 7.75 (d, 1H), 7.68 (t, 1H), 7.44 (s, 1H), 7.30-7.40 (m, 3H), 7.22 (d, 1H), 7.14-7.19 (m, 1H), 3.76 (t, 4H), 3.56 (t, 4H), 3.29-3.34 (m, 4H under water), 3.19 (t, 4H), 2.39 (t, 4H), 1.64-1.73 (m, 2H);

MS m/e MH⁺527.

### Intermediate 12

### {3-[(2-chloropyrimidin-5-yl)ethynyl]phenyl}amine

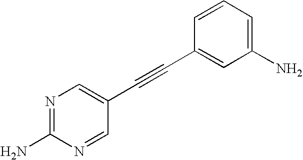

Palladium (10 wt. %) on activated carbon (1.5 g) was added to a stirred solution of 5-bromo-2-chloropyrimidine (12.76 g) and 3-ethynyl aniline (9.28 g) in DIPEA (120 mL) under an inert atmosphere. The reaction mixture was stirred at 80° C. for 4 hours. The reaction mixture was filtered through diatomaceous earth and washed with DCM. The filtrate was purified by flash chromatography on silica using 0-30% EtOAc in DCM as eluent. The resultant solid was triturated with ether to give the title compound as a cream solid (4.28 g, 28%);

¹H NMR (DMSO-d₆) 5.31 (s, 2H), 6.64 (dd, 1H), 6.69-6.76 (m, 2H), 7.08 (dd, 1H), 8.94 (s, 2H);

MS m/e (MH^{+MeCN)}⁺271.

### Intermediate 13

### 5-[(3-aminophenyl)ethynyl]-N-(3-morpholin-4-ylpropyl)pyrimidin-2-amine

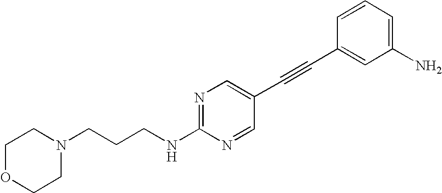

{3-[(2-chloropyrimidin-5-yl)ethynyl]phenyl}amine (Intermediate 12) (0.7 g) and 1-(3-aminopropyl)morpholine (2.22 g) and 1M HCl in diethyl ether were heated at reflux in EtOH (50 mL) for 16 hours. The reaction mixture was concentrated in vacuo and purified by flash chromatography on silica using 0-10% MeOH in DCM as eluent. The product was obtained as a yellow solid (0.35 g);

¹H NMR (DMSO-d₆) 1.67 (m, 2H), 2.28-2.36 (m, 6H), 3.27-3.35 (m, 2H+H₂O), 3.53-3.58 (m, 4H), 5.19 (s, 2H), 6.54-6.58 (m, 1H), 6.60 (d, 1H), 6.66 (s, 1H), 7.01 (t, 1H), 7.62 (t, 1H), 8.66 (s, 2H);

MS m/e MH⁻338.

The following examples were made in a similar way to Example 7 using Intermediate 14 and the appropriate acid:

### EXAMPLE 10

### N-{3-[(2-aminopyrimidin-5-yl)ethynyl]phenyl}-2-fluoro-5-(trifluoromethyl)benzamide

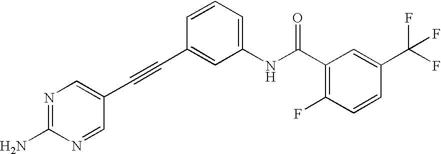

SM: Intermediate 14, 2-fluoro-5-(trifluoromethyl)benzoic acid

¹H NMR (DMSO-d₆) 7.12 (s, 2H), 7.23-7.27 (m, 1H), 7.40 (t, 1H), 7.57-7.62 (m, 2H), 7.95 (s, 1H), 7.96-8.00 (m, 1H), 8.05-8.09 (m, 1H), 8.43 (s, 2H), 10.67 (br s, 1H);

MS m/e MH⁺401.

### EXAMPLE 11

### N-{3-[(2-aminopyrimidin-5-yl)ethynyl]phenyl}-3-methoxybenzamide

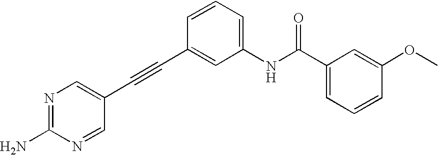

SM: Intermediate 14, 3-methoxybenzoic acid

¹H NMR (DMSO-d₆) 3.83 (s, 3H), 7.12 (s, 2H), 7.13-7.16 (m, 1H), 7.17-7.19 (m, 1H), 3.35-7.55 (m, 4H), 7.72-7.77 (m, 1H), 7.97-8.00 (m, 1H), 8.43 (s, 2H), 10.27 (br s, 1H);

MS m/e MH⁺345.

### EXAMPLE 12

### N-{3-[(2-aminopyrimidin-5-yl)ethynyl]phenyl}-3-isopropoxybenzamide

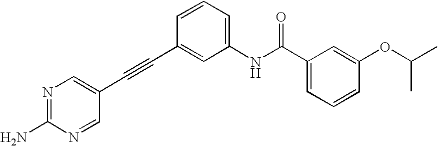

SM: Intermediate 14, 3-isopropoxybenzoic acid

¹H NMR (DMSO-d₆) 1.29 (d, 6H), 4.65-4.77 (m, 1H), 7.12 (s, 2H), 7.14-7.16 (m, 1H), 7.21-7.24 (m, 1H), 7.35-7.51 (m, 4H), 7.73-7.76 (m, 1H), 7.99 (s, 1H), 8.43 (s, 2H), 10.24 (br s, 1H);

MS m/e MH⁺373.

### EXAMPLE 13

### N-{3-[(2-aminopyrimidin-5-yl)ethynyl]phenyl}-3-morpholin-4-ylbenzamide

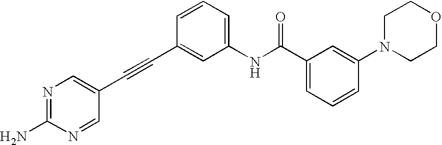

SM: Intermediate 14, 3-morpholin-4-ylbenzoic acid

¹H NMR (DMSO-d₆) 3.15-3.20 (m, 4H), 3.73-3.78 (m, 4H), 7.12 (s, 2H), 7.13-7.17 (m, 1H), 7.20-7.23 (m, 1H), 7.35-7.56 (m, 4H), 7.72-7.77 (m, 1H), 7.96-7.98 (m, 1H), 8.43 (s, 2H), 10.21 (br s, 1H);

MS m/e MH⁺400.

### EXAMPLE 14

### N-{3-[(2-aminopyrimidin-5-yl)ethynyl]phenyl}-3-(1-cyanoethyl)benzamide

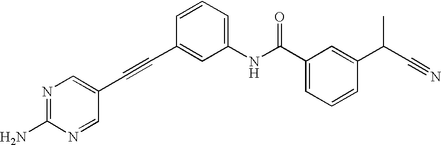

SM: Intermediate 14, 3-(1-cyanoethyl)benzoic acid

¹H NMR (DMSO-d₆) 1.60 (d, 3H), 4.42 (q, 1H), 7.12 (s, 2H), 7.21-7.26 (m, 1H), 7.37-7.43 (m, 1H), 7.57-7.64 (m, 2H), 7.72-7.77 (m, 1H), 7.92-7.98 (m, 3H), 8.43 (s, 2H), 10.37 (br s, 1H);

MS m/e MH⁺368.

### EXAMPLE 15

### N-{3-[(2-aminopyrimidin-5-yl)ethynyl]phenyl}-1,3-benzodioxole-5-carboxamide

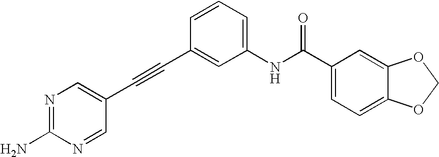

SM: Intermediate 14, piperonylic acid

¹H NMR (DMSO-d₆) 6.13 (s, 2H), 7.03-7.14 (m, 3H), 7.17-7.22 (m, 1H), 7.33-7.39 (m, 1H), 7.49-7.58 (m, 2H), 7.70-7.73 (m, 1H), 7.98 (s, 1H), 8.42 (s, 2H), 10.12 (br s, 1H);

MS m/e MH⁺359.

### EXAMPLE 16

### N-{3-[(2-aminopyrimidin-5-yl)ethynyl]phenyl}-4-[(4-methylpiperazin-1-yl)methyl]benzamide

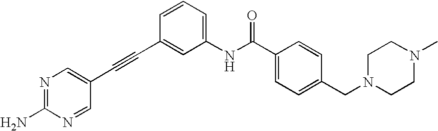

SM: Intermediate 14, 4-(N-methylpiperazinyl)methylbenzoic acid

¹H NMR (DMSO-d₆) 2.18 (s, 3H), 2.33-2.37 (m, 4H), 2.41-2.45 (m, 4H), 3.56 (s, 2H), 6.72 (br s, 2H), 7.21-7.24 (m, 1H), 7.36 (t, 1H), 7.43 (d, 2H), 7.73-7.77 (m, 1H), 7.92 (d, 2H), 7.97-7.99 (m, 1H), 8.42 (s, 2H), 9.98 (br s, 1H);

MS m/e MH⁺427.

### EXAMPLE 17

### N-{3-[(2-aminopyrimidin-5-yl)ethynyl]phenyl}-3-tert-butyl-1-methyl-1H-pyrazole-5-carboxamide

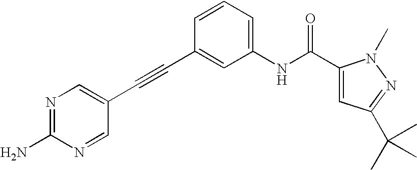

SM: Intermediate 14, 3-tert-butyl-1-methylpyrazole-5-carboxylic acid

¹H NMR (DMSO-d₆) 1.28 (s, 9H), 4.03 (s, 3H), 6.72 (br s, 2H), 6.91 (s, 1H), 7.22-7.25 (m, 1H), 7.36 (t, 1H), 7.68-7.71 (m, 1H), 7.90-7.92 (m, 1H), 8.41 (s, 2H), 9.91 (br s, 1H);

MS m/e MH⁺375.

### EXAMPLE 18

### N-{3-[(2-aminopyrimidin-5-yl)ethynyl]phenyl}-2,2-dimethyltetrahydro-2H-pyran-4-carboxamide

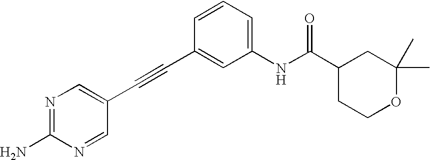

SM: Intermediate 14, 2,2-dimethyl-tetrahydro-2H-pyrane-4-carboxylic acid

¹H NMR (DMSO-d₆) 1.17 (s, 3H), 1.22 (s, 3H), 1.47-1.70 (m, 4H), 2.72-2.80 (m, 1H), 3.58-3.71 (m, 2H), 6.71 (br s, 2H), 7.14-7.17 (m, 1H), 7.30 (t, 1H), 7.51-7.54 (m, 1H), 7.79-7.81 (m, 1H), 8.39 (s, 2H), 9.58 (br s, 1H);

MS m/e MH⁺351.

### Intermediate 14

### 5-[(3-aminophenyl)ethynyl]pyrimidin-2-amine

2-Amino-5-iodopyrimidine (2.21 g), bis(triphenylphosphine)palladium dichloride (350 mg) and copper(I) iodide (40 mg) were stirred in DMF (100 mL)-triethylamine (20 mL) and degassed with nitrogen for 10 min. 3-Ethynyl aniline (1.29 g) was added and the mixture heated to 95° C. for 2 hours. The solvent was evaporated and the residue was purified by trituration with DCM (20 mL) to give the title compound as a brown solid (1.25 g, 60%);

¹H NMR (DMSO-d₆) 5.21 (bs, 2H), 6.58-6.70 (m, 3H), 7.03-7.07 (m, 3H), 8.40 (s, 2H);

MS m/e MH⁺211.

The following examples were made in a similar way to Example 7 by using Intermediate 15 and the appropriate amine:

### EXAMPLE 19

### 2-{3-[(2-aminopyrimidin-5-yl)ethynyl]phenyl}-N-[2-fluoro-5-(trifluoromethyl)benzyl]acetamide

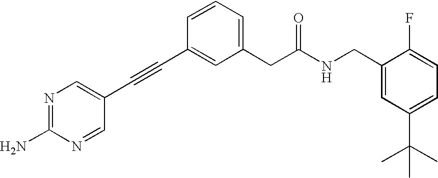

SM: Intermediate 15, 2-fluoro-5-(trifluoromethyl)benzylamine

¹H NMR (DMSO-d₆) 3.53 (s, 2H), 4.40 (d, 2H), 6.71 (br s, 2H), 7.27-7.39 (m, 4H), 7.41-7.43 (m, 1H), 7.60-7.67 (m, 2H), 8.32 (br s, 1H), 8.38 (s, 2H);

MS m/e MH⁺429.

### EXAMPLE 20

### 2-{3-[(2-aminopyrimidin-5-yl)ethynyl]phenyl}-N-(2-morpholin-4-ylbenzyl)acetamide

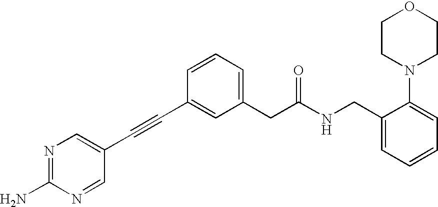

SM: Intermediate 15, (2-morpholin-4-ylbenzyl)amine

¹H NMR (DMSO-d₆) 2.81-2.85 (m, 4H), 3.52 (s, 2H), 3.69-3.73 (m, 4H), 4.40 (d, 2H), 6.71 (br s, 2H), 7.02-7.06 (m, 1H), 7.10-7.13 (m, 1H), 7.20-7.25 (m, 2H), 7.28-7.37 (m, 3H), 7.44 (s, 1H), 7.98 (br s, 1H), 8.38 (s, 2H);

MS m/e MH⁺428.

### Intermediate 15

### {3-[(2-aminopyrimidin-5-yl)ethynyl]phenyl}acetic acid

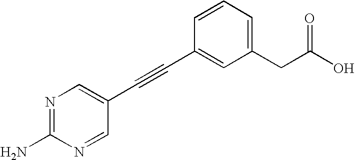

A mixture of 5-ethynylpyrimidin-2-amine (intermediate 2) (1.04 g), 3-iodophenylacetic acid (2.29 g), copper iodide (83 mg) and triethylamine (2.7 mL) in DMF (27 mL) was stirred and degassed with nitrogen. Dichlorobis(triphenylphosphine)palladium(II) (615 mg) was added and the mixture heated at 60° C. for 3 hours. The mixture was cooled to room temperature, then diluted with a 2N aqueous solution of sodium hydroxide (110 mL) and washed with ethyl acetate (70 mL). The aqueous solution was acidified with a 2N aqueous solution of hydrochloric acid. On acidification to pH 4, a brown solid precipitated from the solution. The solid was collected by filtration then dried under vacuum for 20 hours, to give the title compound (2.40 g, 92%).

¹H NMR (DMSO-d₆) 3.59 (s, 2H), 7.11 (br s, 2H), 7.25-7.39 (m, 4H), 8.41 (s, 2H), 12.31 (br s, 1H);

MS m/e MH⁺254.

The Following Examples were made in a Similar way to Example 7 by using Intermediate 16 and the Appropriate Amine:

### EXAMPLE 21

### 3-[(2-aminopyrimidin-5-yl)ethynyl]-N-(2,2-dimethyltetrahydro-2H-pyran-4-yl)benzamide

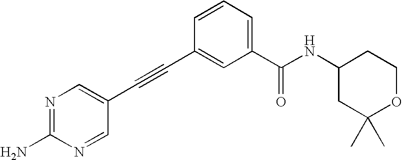

SM: Intermediate 16, 4-amino-2,2-dimethyltetrahydropyran

¹H NMR (DMSO-d₆) 1.18 (s, 3H), 1.23 (s, 3H), 1.40-1.55 (m, 2H), 1.75-1.82 (m, 2H), 3.61-3.72 (m, 2H), 4.13-4.22 (m, 1H), 6.74 (br s, 2H), 7.44-7.49 (m, 1H), 7.59-7.62 (m, 1H), 7.80-7.83 (m, 1H), 7.95-8.00 (m, 2H), 8.42 (s, 2H);

MS m/e MH⁺351.

### EXAMPLE 22

### 3-[(2-aminopyrimidin-5-yl)ethynyl]-N-[2-fluoro-5-(trifluoromethyl)benzyl]benzamide

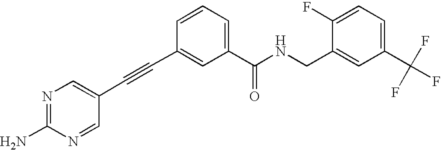

SM: Intermediate 16, 2-fluoro-5-(trifluoromethyl)benzylamine

¹H NMR (DMSO-d₆) 4.40 (d, 2H), 6.74 (br s, 2H), 7.36-7.42 (m, 1H), 7.48-7.52 (m, 1H), 7.62-7.65 (m, 1H), 7.66-7.70 (m, 1H), 7.75-7.78 (m, 1H), 7.85-7.88 (m, 1H), 7.99-8.01 (m, 1H), 8.41 (s, 2H), 8.85-8.90 (m, 1H);

MS m/e MH⁺415.

### EXAMPLE 23

### 3-[(2-aminopyrimidin-5-yl)ethynyl]-N-(3-tert-butyl-1-methyl-1H-pyrazol-5-yl)benzamide

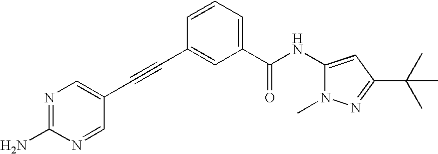

SM: Intermediate 16, 3-tert-butyl-1-methyl-1H-pyrazol-5-amine

¹H NMR (DMSO-d₆) 1.27 (s, 9H), 3.64 (s, 3H), 6.09 (s, 1H), 6.77 (br s, 2H), 7.53-7.57 (m, 1H), 7.68-7.71 (m, 1H), 7.92-7.95 (m, 1H), 8.06-8.08 (m, 1H), 8.43 (s, 2H), 10.01 (br s, 1H);

MS m/e MH⁺375.

### EXAMPLE 24

### 3-[(2-aminopyrimidin-5-yl)ethynyl]-N-(2-morpholin-4-ylbenzyl)benzamide

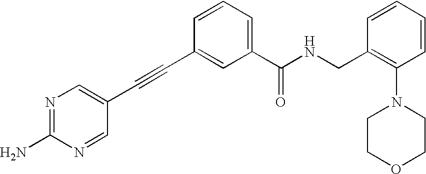

SM: Intermediate 16, (2-morpholin-4-ylbenzyl)amine

¹H NMR (DMSO-d₆) 2.87-2.91 (m, 4H), 3.75-3.79 (m, 4H), 4.63 (d, 2H), 6.74 (br s, 2H), 7.05-7.10 (m, 1H), 7.14-7.17 (m, 1H), 7.22-7.27 (m, 1H), 7.31-7.34 (m, 1H), 7.47-7.51 (m, 1H), 7.61-7.64 (m, 1H), 7.86-7.90 (m, 1H), 8.01-8.03 (m, 1H), 8.42 (s, 2H), 8.63 (m, 1H);

MS m/e MH⁺414.

### Intermediate 16

### 3-[(2-aminopyrimidin-5-yl)ethynyl]benzoic acid

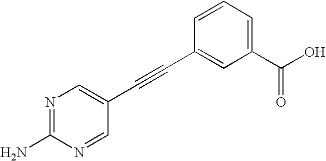

A mixture of 5-ethynylpyrimidin-2-amine (intermediate 2) (2.00 g), 3-iodobenzoic acid (4.16 g), copper iodide (80 mg) and triethylamine (5.2 mL) in DMF (50 mL) was stirred and degassed with nitrogen. Dichlorobis(triphenylphosphine)palladium(II) (590 mg) was added and the mixture heated at 60° C. for 2 hours. The mixture was concentrated, then the residue dissolved in a 1N aqueous solution of sodium hydroxide (350 mL) and washed with ethyl acetate (200 mL). The aqueous solution was acidified with a 2N aqueous solution of hydrochloric acid. On acidification to pH 3, a yellow solid precipitated from the solution. The solid was collected by filtration and washed with water (2×50 mL) then dried under vacuum for 20 hours, to give the title compound (4.27 g, 96%).

¹H NMR (DMSO-d₆) 7.14 (br s, 2H), 7.53 (t, 1H), 7.71 (d, 1H), 7.91 (d, 1H), 8.00 (s, 1H), 8.45 (s, 2H), 13.12 (br s, 1H);

MS m/e MH⁺240.

The Following Examples were made in a Similar way to Example 7 by using Intermediate 17 and the Appropriate Acid:

### EXAMPLE 25

### N-{3-[(2-aminopyrimidin-5-yl)ethynyl]benzyl}benzamide

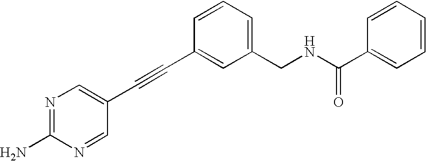

SM: Intermediate 17, benzoic acid

¹H NMR (DMSO-d₆) 4.51 (d, 2H), 6.70 (br s, 2H), 7.34-7.38 (m, 3H), 7.43-7.52 (m, 4H), 7.87-7.91 (m, 2H), 8.39 (s, 2H), 8.69 (m, 1H);

MS m/e MH⁺329.

### EXAMPLE 26

### N-{3-[(2-aminopyrimidin-5-yl)ethynyl]benzyl}-3-tert-butyl-1-methyl-1H-pyrazole-5-carboxamide

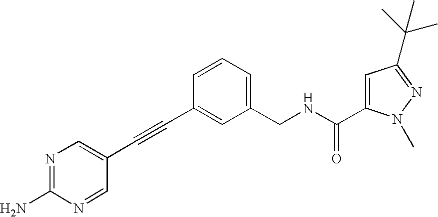

SM: Intermediate 17, 3-tert-butyl-1-methyl-1H-pyrazole-5-carboxylic acid

¹H NMR (DMSO-d₆) 1.27 (s, 9H), 4.00 (s, 3H), 4.43 (d, 2H), 5.65 (s, 1H), 6.70 (br s, 2H), 7.32-7.39 (m, 3H), 7.45 (s, 1H), 8.39 (s, 2H), 8.56-8.81 (m, 1H);

MS m/e MH⁺389.

### Intermediate 17

### 5-{[3-(aminomethyl)phenyl]ethynyl}pyrimidin-2-amine

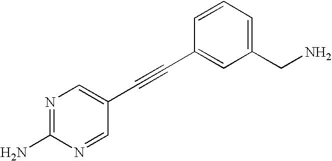

A mixture of 5-ethynylpyrimidin-2-amine (intermediate 2) (2.40 g), 3-iodobenzylamine (4.89 g), copper iodide (96 mg) and triethylamine (3.4 mL) in DMF (50 mL) was stirred and degassed with nitrogen. Dichlorobis(triphenylphosphine)palladium(II) (705 mg) was added and the mixture heated at 60° C. for 2 hours. The mixture was concentrated in vacuo, then water (100 mL) was added, and the mixture slurried for 10 min, then the solid was collected by filtration. Purification by flash chromatography on silica using 5-10% 2N NH₃/MeOH in DCM as eluent afforded the title compound as a yellow solid (2.48 g, 55%);

¹H NMR (DMSO-d₆) 1.83 (br s, 2H), 3.72 (s, 2H), 7.09 (br s, 2H), 7.31-7.33 (m, 3H), 7.47 (s, 1H), 8.40 (s, 2H);

MS m/e MH⁺225.

### EXAMPLE 27

### N-{3-[(2-aminopyrimidin-5-yl)ethynyl]-4-methoxyphenyl}-3-[4-(trifluoromethyl)phenyl]propanamide

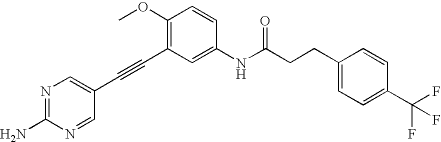

5-(5-Amino-2-methoxy-phenylethynyl)-pyrimidin-2-ylamine (Intermediate 19) (0.17 g), 3-[4-(trifluoromethyl)phenyl]propionic acid (0.17 g), HATU (0.32 g) and DIPEA (0.25 mL) were added to DMF (6 mL) and stirred at room temperature for 1 hour. Water (10 mL) was added and the solid was filtered. Purification by flash chromatography on silica using 2.5-5.0% MeOH in DCM as eluent gave the title compound as a white solid (119 mg, 39%);

¹H NMR (300.132 MHz, DMSO) □ 9.85 (s, 1H), 8.39 (s, 2H), 7.72 (s, 1H), 7.65 (d, 2H), 7.50-7.43 (m, 3H), 7.10 (s, 2H), 7.01 (d, 1H), 3.81 (s, 3H), 3.01 (t, 2H), 2.65 (t, 2H);

MS m/e MH⁺441.

### Intermediate 18

### 5-(2-Methoxy-5-nitro-phenylethynyl)-pyrimidin-2-ylamine

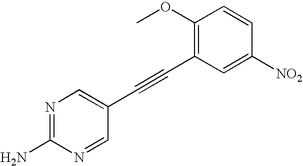

A mixture of 4-iodo-2-nitroanisole (5.0 g), 5-ethynylpyrimidin-2-amine (Intermediate 2) (2.3 g), copper (I) iodide (27 mg) and bis(triphenylphosphine)palladium (II) chloride (176 mg) in DMF (90 mL) was stirred under an inert atmosphere. TEA (18 mL) was added and the reaction was stirred at 60° C. for 3 hours. The reaction was filtered and the DMF was removed in vacuo to give a black gum/solid. DCM (100 mL) was added and the orange solid was filtered and dried (3.2 g, 67%);

¹H NMR (300.074 MHz, dmso) □ 8.43 (s, 2H), 8.25-8.22 (m, 2H), 7.29 (d, 1H), 7.18 (s, 2H), 3.99 (s, 3H); MS m/e MH⁺271.

### Intermediate 19

### 5-(5-Amino-2-methoxy-phenylethynyl)-pyrimidin-2-ylamine

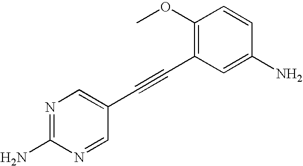

A mixture of 5-(2-Methoxy-5-nitro-phenylethynyl)-pyrimidin-2-ylamine (Intermediate 18) (3.2 g), indium powder (5.4 g) and conc HCl (1.8 mL) was stirred in THF/water (75 mL/225 mL) overnight. The reaction was basified with aqueous potassium carbonate and the slurry was filtered through a pad of diatomaceous earth, the filtrate was extracted with EtOAc (3×200 mL), dried (MgSO₄) and the solvent removed in vacuo to yield a black solid. Purification by flash chromatography on silica using 0-2.5% MeOH in DCM as eluent gave the title compound as a white solid (1.2 g, 42%);

¹H NMR (300.074 MHz, dmso) □ 8.34 (s, 2H), 7.04 (s, 2H), 6.77 (d, 1H), 6.66 (s, 1H), 6.58 (d, 1H), 4.78 (s, 2H), 3.70 (s, 3H);

MS m/e MH⁺241.

### EXAMPLE 28

### N-{5-[(2-aminopyrimidin-5-yl)ethynyl]-6-methylpyridin-3-yl}-3-[4-(trifluoromethyl)phenyl]propanamide

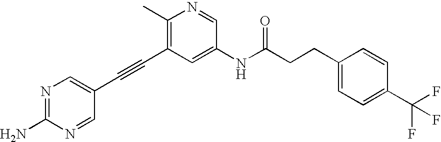

5-(5-Amino-2-methyl-pyridin-3-ylethynyl)-pyrimidin-2-ylamine (Intermediate 21) (0.17 g), 3-[4-(trifluoromethyl)phenyl]propionic acid (0.22 g), HATU (0.38 g) and DIPEA (0.27 mL) were added to DMF (6 mL) and stirred at room temperature for 1 hour. Water (10 mL) was added and the solid was filtered. Purification by flash chromatography on silica using 2.5-5.0% MeOH in DCM as eluent gave the title compound as a white solid (182 mg, 56%);

¹H NMR (300.132 MHz, DMSO) □ 10.17 (s, 1H), 8.48 (s, 3H), 8.15 (s, 1H), 7.66 (d, 2H), 7.49 (d, 2H), 7.19 (s, 2H), 3.02 (t, 2H), 2.71 (t, 2H), 2.57 (s, 3H);

MS m/e MH⁺426.

### Intermediate 20

### 5-(2-Methyl-5-nitro-pyridin-3-ylethynyl)-pyrimidin-2-ylamine

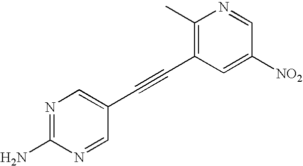

A mixture of 3-nitro-5-bromo-6-methylpyridine (5.0 g), 5-ethynylpyrimidin-2-amine (Intermediate 2) (3.0 g), copper (I) iodide (18 mg) and bis(triphenyl phosphine)palladium (II) chloride (226 mg) in DMF (90 mL) was stirred under an inert atmosphere. TEA (18 mL) was added and the reaction was stirred at 60° C. for 3 hours. The reaction was cooled and filtered (3.2 g, 54%);

¹H NMR (300.074 MHz, dmso) □ 9.18 (s, 1H), 8.56 (s, 1H), 8.52 (s, 2H), 7.28 (s, 2H), 2.76 (s, 3H);

MS m/e MH⁺256.

### Intermediate 21

### 5-(5-Amino-2-methyl-pyridin-3-ylethynyl)-pyrimidin-2-ylamine

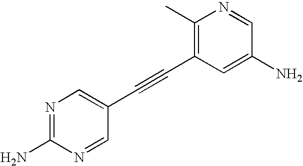

5-(2-Methyl-5-nitro-pyridin-3-ylethynyl)-pyrimidin-2-ylamine (Intermediate 20) (3.2 g) and iron powder (2.1 g) were added to glacial acetic acid (90 mL) and heated at 60° C. for 3 hours. The reaction was filtered and the solvent removed in vacuo to give a black, viscous oil. Acetone (100 mL) and water (100 mL) were added to the viscous oil, this was then basified with potassium carbonate, a portion of hot EtOAc (200 mL) was added and the residue was passed through a plug of diatomaceous earth. The filtrate was extracted with hot EtOAc (3×200 mL), dried (MgSO₄) and the solvent removed in vacuo to yield a off white solid (1.2 g, 43%);

¹HNMR (300.074 MHz, dmso) □ 8.41 (s, 2H), 7.81 (s, 1H), 7.12 (s, 2H), 6.96 (s, 1H), 5.19 (s, 2H), 2.42 (s, 3H);

MS m/e MH⁺226.

### EXAMPLE 29

### N-{3-[(2-aminopyrimidin-5-yl)ethynyl]-2-methoxyphenyl}-3-[4-(trifluoromethyl)phenyl]propanamide

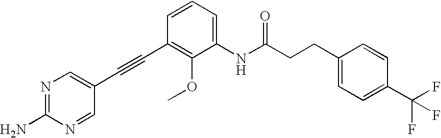

5-(5-Amino-6-methoxy-phenylethynyl)-pyrimidin-2-ylamine (Intermediate 23) (0.17 g), 3-[4-(trifluoromethyl)phenyl]propionic acid (0.20 g), HATU (0.35 g) and DIPEA (0.33 mL) were added to DMF (6 mL) and stirred at room temperature for 1 hour. Water (10 mL) was added and the solid was filtered. Purification by flash chromatography on silica using 2.5-5.0% MeOH in DCM as eluent gave the title compound as a white solid (123 mg, 39%);

¹H NMR (300.132 MHz, DMSO) □ 9.36 (s, 1H), 8.44 (s, 2H), 7.99 (d, 1H), 7.66 (d, 2H), 7.51 (d, 2H), 7.20 (d, 1H), 7.15 (s, 2H), 7.07 (t, 1H), 3.84 (s, 3H), 3.02 (t, 2H), 2.81 (t, 2H);

MS m/e MH⁺441.

### Intermediate 22

### 5-(6-Methoxy-5-nitro-phenylethynyl)-pyrimidin-2-ylamine

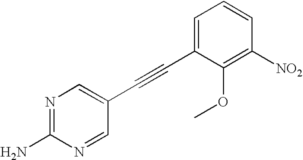

A mixture of 1-iodo-2-methoxy-3-nitro-benzene (5.0 g), 5-ethynylpyrimidin-2-amine (Intermediate 2) (2.1 g), copper (I) iodide (27 mg) and bis(triphenylphosphine) palladium (II) chloride (176 mg) in DMF (90 mL) was stirred under an inert atmosphere. TEA (18 mL) was added and the reaction was stirred at 60° C. for five hours. The DMF was removed in vacuo to yield a black gum/solid. DCM (100 mL) was added and the solid was filtered and dried (3.2 g, 65%);

¹H NMR (300.074 MHz, dmso) □ 8.47 (s, 2H), 7.87 (d, 1H), 7.79 (d, 1H), 7.33 (t, 1H), 7.23 (s, 2H), 4.05 (s, 3H);

MS m/e MH⁺271.

### Intermediate 23

### 5-(5-Amino-6-methoxy-phenylethynyl)-pyrimidin-2-ylamine

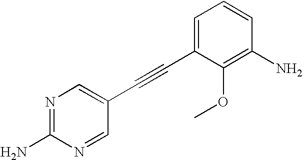

A mixture of 5-(6-methoxy-5-nitro-phenylethynyl)-pyrimidin-2-ylamine (Intermediate 22) (3.2 g) and iron powder (2.0 g) was stirred at 60° C. for 7 hours in glacial acetic acid (50 mL). The reaction was filtered and the solvent removed in vacuo to yield a black tar. Water (70 mL) and 10% MeOH/EtOAc (100 mL) were added then basified with potassium carbonate, extracted with hot 10% MeOH/EtOAc (3×200 mL), dried (MgSO₄) and the solvent removed in vacuo to yield a yellow solid (2.2 g, 76%);

¹H NMR (300.074 MHz, dmso) □ 8.38 (s, 2H), 7.08 (s, 2H), 6.80 (t, 1H), 6.69 (d, 1H), 6.61 (d, 1H), 5.02 (s, 2H), 3.80 (s, 3H);

MS m/e MH⁺241.

### EXAMPLE 30

### N-{5-[(2-aminopyrimidin-5-yl)ethynyl]-2-methylpyridin-3-yl}-3-[4-(trifluoromethyl)phenyl]propanamide

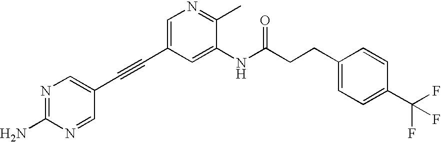

5-(5-Amino-6-methyl-pyridin-3-ylethynyl)-pyrimidin-2-ylamine (Intermediate 27) (0.17 g), 3-[4-(trifluoromethyl)phenyl]propionic acid (0.22 g), HATU (0.38 g) and DIPEA (0.27 mL) were added to DMF (6 mL) and stirred at room temperature for 1 hour. Water (10 mL) was added and the solid was filtered. The solid was added to 50% MeOH/DCM and sonicated for 20 minutes, the title compound was filtered and dried (129 mg, 40%);

¹H NMR (300.132 MHz, DMSO) □ 9.50 (s, 1H), 8.45 (s, 2H), 8.36 (s, 1H), 7.94 (s, 1H), 7.67 (d, 2H), 7.51 (d, 2H), 7.17 (s, 2H), 3.03 (t, 2H), 2.77 (t, 2H), 2.35 (s, 3H);

MS m/e MH⁺426.

### Intermediate 24

### 2-(5-Bromo-3-nitro-pyridin-2-yl)-malonic acid diethyl ester

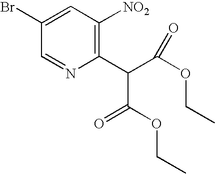

Sodium hydride (4.0 g) was dissolved in DMF (60 mL) to this was slowly added diethylmalonate (12.7 g) under an inert atmosphere. Once the addition was complete the reaction was stirred for 10 minutes. 5-Bromo-2-chloro-3-nitro-pyridine (10 g) in DMF (20 mL) was slowly added to the anion, the reaction was maintained at approximately 40° C. for 1 hour before being quenched with water (100 mL), extracted with diethyl ether (3×250 mL), dried (MgSO₄) and solvent removed in vacuo to yield a yellow oil (12.6 g, 86%);

¹H NMR (300.072 MHz, cdcl3) □ 8.87 (s, 1H), 8.62 (s, 1H), 5.46 (s, 1H), 4.30 (q, 4H), 1.29 (t, 6H);

MS m/e MH⁺352.

### Intermediate 25

### 5-Bromo-2-methyl-3-nitro-pyridine

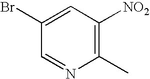

2-(5-Bromo-3-nitro-pyridin-2-yl)-malonic acid diethyl ester (Intermediate 24) (12.6 g) was added to 7.0 N HCl (100 mL) and heated to reflux for 5 hours, the reaction was cooled, extracted with diethyl ether (3×250 mL), dried (MgSO₄) and solvent removed in vacuo to yield a yellow oil. Purification by flash chromatography on silica using 20% diethylether in iso-hexane as eluent gave the title compound as a yellow waxy solid (7.5 g, 96%);

¹H NMR (300.072 MHz, cdcl3) □ 8.79 (s, 1H), 8.43 (s, 1H), 2.83 (s, 3H).

### Intermediate 26

### 5-(6-Methyl-5-nitro-pyridin-3-ylethynyl)-pyrimidin-2-ylamine

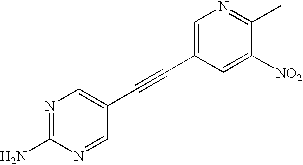

A mixture of 5-bromo-2-methyl-3-nitro-pyridine (Intermediate 25) (5.0 g), 5-ethynylpyrimidin-2-amine (Intermediate 2) (3.0 g), copper (I) iodide (34 mg) and bis(triphenyl phosphine)palladium (II) chloride (225 mg) in DMF (90 mL) was stirred under an inert atmosphere. TEA (18 mL) was added and the reaction was stirred at 60° C. for three hours. The reaction was allowed to cool and filtered, the filtrate was removed in vacuo to yield a black solid, DCM (100 mL) was added and the slurry filtered to yield a brown solid (6.7 g), impure sample;

¹H NMR (300.074 MHz, dmso) □ 8.86 (s, 1H), 8.49-8.47 (m, 3H), 7.25 (s, 2H), 2.77 (s, 3H); MS m/e MH⁺256.

### Intermediate 27

### 5-(5-Amino-6-methyl-pyridin-3-ylethynyl)-pyrimidin-2-ylamine

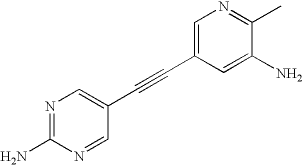

5-(6-Methyl-5-nitro-pyridin-3-ylethynyl)-pyrimidin-2-ylamine (Intermediate 26) (6.7 g) and iron powder (2.6 g) were added to glacial acetic acid (90 mL) and heated at 60° C. for 1 hour. The reaction was filtered and the solvent removed in vacuo to yield a viscous oil. Acetone (100 mL), water (100 mL) and methanol (30 mL) were added to the viscous oil, this was then basified with potassium carbonate, extracted with hot 10% MeOH/EtOAc (3×200 mL), dried (MgSO₄) and solvent removed in vacuo to yield a brown solid (2.6 g, 50% over two steps);

¹H NMR (300.074 MHz, dmso) □ 8.39 (s, 2H), 7.78 (s, 1H), 7.10 (s, 2H), 6.98 (s, 1H), 5.20 (s, 2H), 2.27 (s, 3H);

MS m/e MH⁺226.

### EXAMPLE 31

### N-{5-[(2-aminopyrimidin-5-yl)ethynyl]-2-methoxyphenyl]-3-[4-(trifluoromethyl)phenyl]propanamide

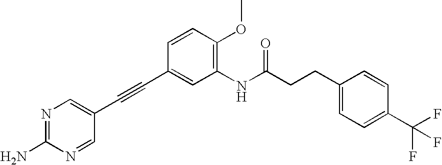

5-(5-Amino-4-methoxy-phenylethynyl)-pyrimidin-2-ylamine (Intermediate 29) (0.17 g), 3-[4-(trifluoromethyl)phenyl]propionic acid (0.19 g), HATU (0.35 g) and DIPEA (0.33 mL) were added to DMF (6 mL) and stirred at room temperature for 1 hour. Water (10 mL) was added and the solid was filtered. Purification by flash chromatography on silica using 2.5-10% MeOH in DCM as eluent gave the title compound as a white solid (159 mg, 51%);

¹H NMR (300.132 MHz, DMSO) □ 9.22 (s, 1H), 8.41 (s, 2H), 8.12 (s, 1H), 7.64 (d, 2H), 7.51 (d, 2H), 7.21 (s, 1H), 7.06 (s, 3H), 3.85 (s, 3H), 3.06-2.94 (m, 2H), 2.85-2.71 (m, 2H);

MS m/e MH⁺441.

### Intermediate 28

### 5-(4-Methoxy-5-nitro-phenylethynyl)-pyrimidin-2-ylamine

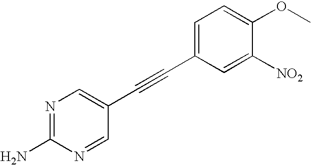

A mixture of 1-bromo-4-methoxy-3-nitro-benzene (5.0 g), 5-ethynylpyrimidin-2-amine (Intermediate 2) (2.8 g), copper (I) iodide (33 mg) and bis(triphenylphosphine)palladium (II) chloride (212 mg) in DMF (90 mL) was stirred under an inert atmosphere. TEA (18 mL) was added and the reaction was stirred at 60° C. for 16 hours. The DMF was removed in vacuo to give a black solid. DCM (100 mL) was added and the solid was filtered and dried (10 g, impure); MS m/e MH⁺271.

### Intermediate 29

### 5-(5-Amino-4-methoxy-phenylethynyl)-pyrimidin-2-ylamine

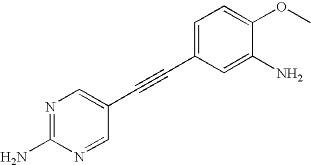

A mixture of 5-(4-methoxy-5-nitro-phenylethynyl)-pyrimidin-2-ylamine (Intermediate 28) (10 g) and iron powder (4.7 g) was stirred at 60° C. for five hours in glacial acetic acid (50 mL). The reaction was filtered and the filtrate was removed in vacuo to yield a yellow solid. Purification by flash chromatography on silica using 2.5-10% MeOH in DCM as eluent gave the title compound as a yellow solid (2.5 g, 48% over two steps);

¹H NMR (300.074 MHz, dmso) 68.35 (s, 2H), 7.00 (s, 2H), 6.79 (d, 1H), 6.74 (s, 1H), 6.69 (d, 1H), 4.86 (brs, 2H), 3.77 (s, 3H);

MS m/e MH⁺241.

The Following Example was made in a Similar way to Example 7 using Intermediate 17 and the Appropriate Acid.

### EXAMPLE 32

### N-[[3-[2-(2-aminopyrimidin-5-yl)ethynyl]phenyl]methyl]-3-(trifluoromethyl)benzamide

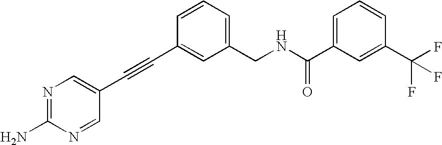

SM: Intermediate 17, 3-(trifluoromethyl)benzoic acid

¹H NMR (DMSO-d₆) □ 4.53 (d, 2H), 6.71 (br s, 2H), 7.40-7.35 (m, 3H), 7.49 (s, 1H), 7.71 (t, 1H), 7.86 (d, 1H), 8.22-8.17 (m, 2H), 8.39 (s, 2H), 8.99 (br s, 1H);

MS m/e MH⁺397.

The Following Example was made in a Similar way to Example 9 using Intermediate 31 and the Appropriate Acid.

### EXAMPLE 33

### 3-(1-cyanoethyl)-N-[6-methyl-5-[2-[2-(2-morpholin-4-ylethylamino)pyrimidin-5-yl]ethynyl]pyridine-3-yl]-benzamide

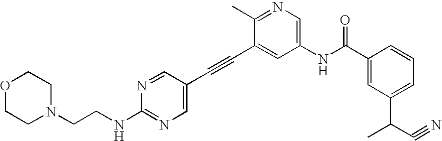

SM: [5-(5-Amino-2-methyl-pyridin-3-ylethynyl)-pyrimidin-2-yl]-(2-morpholin-4-yl-ethyl)-amine (Intermediate 31) and 3-(1-cyanoethyl)benzoic acid.

¹H NMR (DMSO-d₆) 10.5 (s, 1H), 8.75 (s, 1H), 8.5 (s, 2H), 8.3 (s, 1H), 8.0 (m, 2H), 7.6 (m, 3H), 4.45 (m, 1H), 3.6 (m, 4H), 3.45(m, 2H), 2.6 (s, 3H), 2.4 (m, 4H), 1.6 (d, 3H).

MS m/e MH⁺496.

### Intermediate 31

### [5-(5-Amino-2-methyl-pyridin-3-ylethynyl)-pyrimidin-2-yl]-(2-morpholin-4-yl-ethyl)-amine

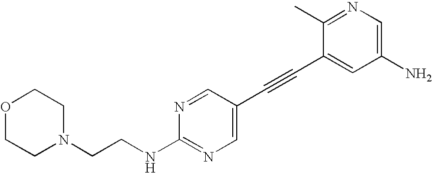

(5-Bromo-pyrimidin-2-yl)-(2-morpholin-4-yl-ethyl)-amine (Intermediate 32) (7.0 g), PdCl₂(PPh₃)₂ (0.17 g), triphenylphosphine (0.2 g) and 5-ethynyl-6-methyl-pyridin-3-ylamine (Intermediate 41—see below) (3.54 g) were added to piperidine (70 mL) and heated at reflux for 2 hours. The piperidine was then removed in vacuo to yield a yellow gum. This was dissolved in water (200 mL), extracted with DCM (2×250 mL), dried (MgSO₄) and solvent removed in vacuo to yield a yellow gum. The gum was dissolved in hot DCM, trituration with diethyl ether precipitated a yellow solid. Purification of the remaining liquor was achieved by flash chromatography on silica using 2.5-10% MeOH in DCM as eluent gave the title compound as a yellow solid (6.5 g, 78%);

¹H NMR (300.132 MHz, CDCl3) δ 8.41 (s, 2H), 7.00 (d, 1H), 6.81 (s, 1H), 6.59 (d, 1H), 5.84 (brs, 1H), 3.72 (t, 4H), 3.56-3.50 (m, 4H), 2.60 (t, 2H), 2.49 (t, 4H), 2.36 (s, 3H);

MS m/e MH⁺339.

### Intermediate 32

### (5-Bromo-pyrimidin-2-yl)-(2-morpholin-4-yl-ethyl)-amine

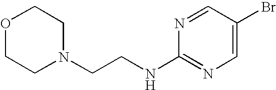

2-Chloro-5-bromopyrimidine (100 g) was added to propan-2-ol (700 mL), DIPEA (184 mL) and aminoethylmorpholine (80.8 g), then heated at 80° C. for 7 hours. The reaction was allowed to cool and the solvent removed in vacuo to yield an orange gum, this was quenched with water (200 mL), extracted with diethyl ether (3×600 mL), dried (MgSO₄) and solvent removed in vacuo to yield a yellow viscous oil. Trituration with diethyl ether (100 mL) gave a solid, which was stirred in iso-hexane (200 mL) for 20 mins before being filtered and dried. White solid obtained (118 g, 79%);

¹H NMR (300.072 MHz, cdcl3) δ 8.28 (s, 2H), 5.73 (s, 1H), 3.72 (t, 4H), 3.46 (q, 2H), 2.59 (t, 2H), 2.49 (t, 4H);

MS m/e MH⁺288.

The following example was made in a similar way to Example 27 using the appropriate amine intermediate and the appropriate acid and using RP-HPLC for purification.

### EXAMPLE 34

### N-[4-methyl-3-[2-[2-(3-morpholin-4-ylpropylamino)pyrimidin-5-yl]ethynyl]phenyl]-3-(trifluoromethyl)benzamide

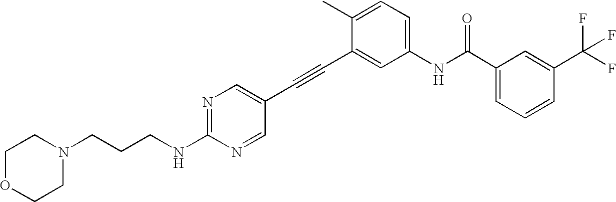

SM: 5-[2-(5-amino-2-methyl-phenyl)ethynyl]-N-(3-morpholin-4-ylpropyl)pyrimidin-2-amine (Intermediate 36, 3-(trifluoromethyl)benzoic acid

¹H NMR (DMSO-d₆) δ10.45 (s, 1H), 8.49 (s, 2H), 8.31 (s, 1H), 8.27 (d, 1H), 7.98 (d, 1H), 7.95 (s, 1H), 7.80 (t, 1H), 7.70 (t, 1H), 7.66 (d, 1H), 7.31 (d, 1H), 3.57 (t, 4H), 3.35 (q, 2H), 2.43 (s, 3H), 2.36-2.32 (m, 6H), 1.70 (quintet, 2H)

MS m/e MH⁺524.

### Intermediate 33

### (5-Bromo-pyrimidin-2-yl)-(3-morpholin-4-yl-propyl)-amine

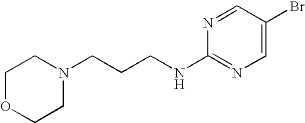

2-Chloro-5-bromopyrimidine (50 g) was added to propan-2-ol (300 mL), DIPEA (92 mL) and aminopropylmorpholine (46 mL) and the reaction was heated at 80° C. for 3 hours. The reaction was allowed to cool and the solvent removed in vacuo to yield an orange gum, this was quenched with water (200 mL), extracted with diethyl ether (3×600 mL), dried (MgSO₄) and solvent removed in vacuo to yield a yellow viscous oil. Diethyl ether (100 mL) was rapidly added to the gum with scratching until a solid crashed out, this was filtered. Process repeated on the filtered mother liquor until no solid crashed out. The obtained solids were combined and stirred in iso-hexane (200 mL) for 20 mins before being filtered and dried. White solid obtained (67 g, 86%);

¹H NMR (CDCl₃) δ 8.25 (s, 2H), 6.02 (s, 1H), 3.73 (t, 4H), 3.45 (q, 2H), 2.49-2.44 (m, 6H), 1.78 (quintet, 2H);

MS m/e MH⁺302.

### Intermediate 34

### 4-(5-Amino-2-methyl-phenyl)-2-methyl-but-3-yn-2-ol

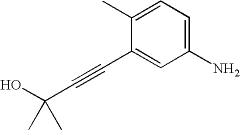

3-Iodo-4-methylaniline (100 g), bis(triphenylphosphine)palladium (II) chloride (6.0 g), triphenylphosphine (112 g) and 2-methyl-but-3-yn-2-ol (83 mL) were added to piperidine (600 mL) and stirred at reflux under an inert atmosphere for 4 hours. The piperidine was removed in vacuo to afford a viscous black sludge. The slurry was stirred in diethyl ether (300 mL) before being acidified with aqueous citric acid (500 mL). The aqueous was washed with another portion of diethyl ether (150 mL), the ether layers were combined and re-extracted with aqueous citric acid (500 mL), the combined aqueous layer was then basified with potassium carbonate, extracted with diethyl ether (3×500 mL), dried (MgSO₄) and the solvent removed in vacuo to yield a black viscous oil. The oil was dissolved in 80% diethyl ether/iso-hexane and passed down a 4 inch plug of silica eluting with 80% diethyl ether/iso-hexane. On removal of the solvent an orange oil was obtained which solidified overnight (87 g);

MS m/e MH⁺190.

### Intermediate 35

### 3-Ethynyl-4-methyl-phenylamine

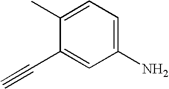

4-(5-Amino-2-methyl-phenyl)-2-methyl-but-3-yn-2-ol (Intermediate 34) (81 g) was added to toluene, then powdered NaOH (25.8 g) and the reaction was heated at reflux for 6 hours. The toluene was removed in vacuo, aqueous NaHCO₃ (300 mL) added, extracted with diethyl ether (3×400 mL), dried (MgSO₄) and the solvent removed in vacuo to yield a black oil. Compound purified via bulb-to-bulb distillation at 0.30 mbar @ 120° C. Slightly yellow oil obtained (47 g, 84% over two steps);

¹H NMR (300.072 MHz, cdcl3) δ 6.97 (d, 1H), 6.80 (s, 1H), 6.60 (d, 1H), 3.53 (brs, 1H), 3.20 (s, 1H), 2.33 (s, 3H).

### Intermediate 36

### 5-[2-(5-amino-2-methyl-phenyl)ethynyl]-N-(3-morpholin-4-ylpropyl)pyrimidin-2-amine

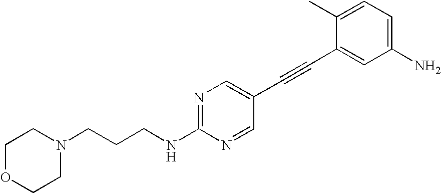

Intermediate 33 (15 g, 49.8 mmol), PdCl₂(PPh₃)₂ (0.35 g, 0.50 mmol), PPh₃ (1.3 g, 4.98 mmol) and 3-ethynyl-4-methyl-phenylamine (intermediate 35) (6.8 g, 52.3 mmol) were added to piperidine (150 ml) and heated at reflux for 2 hours. The piperidine was then removed in vacuo to yield a yellow solid. This solid was dissolved in water (200 ml) and the aqueous was extracted with DCM (3×250 ML), dried (MgSO₄) and solvent removed in vacuo to yield a yellow solid. This solid was recrystalised from DCM/ether to give a solid. The remaining solvate was purified by flash chromatography on silica eluting with DCM, 2.5% MeOH/DCM, 5% MeOH/DCM and finally 10% MeOH/DCM. Both solids were combined to give the title compound as a yellow solid (14.6 g);

¹H NMR (CDCl₃) □ 8.40 (s, 2H), 7.00 (d, 1H), 6.81 (s, 1H), 6.59 (d, 1H), 6.14 (t, 1H), 3.74 (t, 4H), 3.61-3.49 (m, 4H), 2.50-2.45 (m, 6H), 2.36 (s, 3H), 1.80 (quintet, 2H);

MS m/e MH⁺352.

### EXAMPLE 35

### N-[5-[2-(2-aminopyrimidin-5-yl)ethynyl]pyridin-3-yl]-3-morpholin-4-yl-benzamide

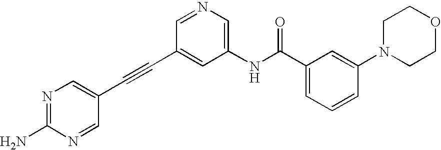

DIPEA (387 mg) was added to a mixture of 5-[(5-aminopyridin-3-yl)ethynyl]pyrimidin-2-amine (Intermediate 11) (211 mg), 3-morpholin-4-ylbenzoic acid (414 mg) and HBTU (642 mg) in DMF (3 mL) and stirred for 65 hours at ambient temperature. The mixture was purified by RPHPLC. Product fractions were made basic with aqueous ammonia and concentrated to low volume. The solid formed was filtered off and dried to give the product (179 mg, 44%).

¹H NMR (DMSO-d₆) 3.20 (m, 4H), 3.76 (m, 4H), 7.18 (m, 3H), 7.39 (m, 2H), 7.46 (m, 1H), 8.33 (m, 1H), 8.42 (m, 1H), 8.49 (s, 2H), 8.87 (m, 1H), 10.42 (s, 1H);

MS m/e MH⁺401.

### EXAMPLE 36

### 3-[2-(2-aminopyrimidin-5-yl)ethynyl]-N-(1-propan-2-ylpyrazol-3-yl)-benzamide

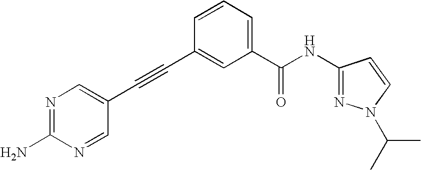

DIPEA (129 mg) was added to a mixture of 3-[(2-aminopyrimidin-5-yl)ethynyl]benzoic acid (Intermediate 16) (119 mg), 3-amino-1-isopropylpyrazole (Intermediate 37) (63 mg) and HATU (379 mg) in DMF (1.5 mL) and stirred for 17 hours at ambient temperature. The mixture was diluted with 1M aqueous sodium hydroxide and extracted with EtOAc. The extract was washed with brine, dried, and the solvent evaporated. Purification of the residue by RPHPLC gave the product as a solid (11 mg, 6%).

MS m/e MH⁺347.

### Intermediate 37

### 3-Amino-1-isopropylpyrazole

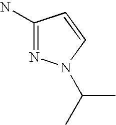

Isopropylhydrazine hydrochloride (743 mg) was stirred with potassium carbonate (2.76 g) in water (15 mL) and 2-chloroacrylonitrile (870 mg) was added. The mixture was heated at 50° C. for 1 hour and then extracted with EtOAc. The extract was washed with brine, dried and the solvent evaporated to give crude product as a brown oil (630 mg, 74%) which was used in subsequent procedures without further purification;

¹H NMR (DMSO-d₆) 1.32 (d, 6H), 4.17 (m, 1H), 4.51 (s, br, 2H), 5.34 (d, 1H), 7.30 (d, 1H).

The Following Example was made in a Similar way to Example 36 using Intermediate 16 and the Appropriate Amine.

### EXAMPLE 37

### 3-[2-(2-aminopyrimidin-5-yl)ethynyl]-N-(5-cyclopropyl-2-methyl-pyrazol-3-yl)-benzamide

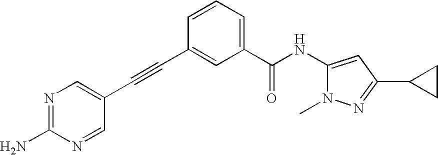

SM: Intermediate 16, 5-cyclopropyl-2-methyl-pyrazole-3-amine.

MS m/e MH⁺359.

The Following Examples were made in a Similar way to Example 7 Using the Appropriate Amines and Acids.

### EXAMPLE 38

### 2-methyl-N-[3-[2-[4-(3-morpholin-4-ylpropylamino)phenyl]ethynyl]phenyl]-5-tert-butyl-pyrazole-3-carboxamide

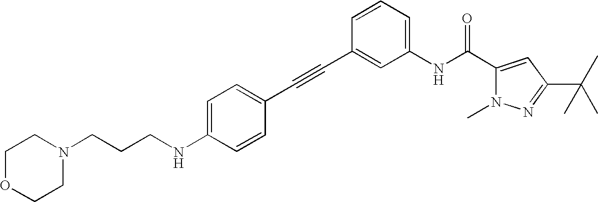

SM:

5-[(3-aminophenyl)ethynyl]-N-(3-morpholin-4-ylpropyl)pyrimidin-2-amine (Intermediate 13), 2-methyl-5-tert-butyl pyrazole-3-carboxylic acid.

¹H NMR (DMSO-d₆) 1.27 (s, 9H), 1.61-1.76 (m, 2H), 2.27-2.38 (m, 6H), 3.23-3.39 (m, 2H under water peak), 3.55 (t, 4H), 4.02 (s, 3H), 6.95 (s, 1H), 7.22 (d, 1H), 7.38 (t, 1H), 7.67 (d, 2H), 7.95 (s, 1H), 8.39-8.52 (br s, 2H), 10.20 (s, 1H);

MS m/e MH⁺502.

### EXAMPLE 39

### 3-(1-cyanoethyl)-N-[3-[2-[2-(3-morpholin-4-ylpropylamino)pyrimidin-5-yl]ethynyl]phenyl]-benzamide

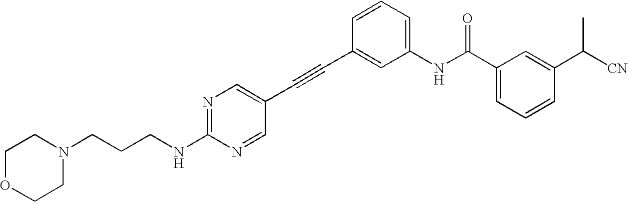

SM: 5-[(3-aminophenyl)ethynyl]-N-(3-morpholin-4-ylpropyl)pyrimidin-2-amine (Intermediate 13), 3-(1-cyanoethyl)benzoic acid.

¹H NMR (DMSO-d₆) 1.60 (d, 3H), 1.63-1.75 (m, 2H), 1.18-2.39 (m, 6H), 3.24-3.37 (m, 4H under water peak), 3.56 (t, 2H), 4.42 (q, 1H), 7.24 (d, 1H), 7.39 (t, 1H), 7.54-7.78 (m, 4H), 7.93 (d, 2H), 7.97 (s, 1H), 8.40-8.53 (br s, 2H), 10.36 (s, 1H);

MS m/e MH⁺495.

### EXAMPLE 40

### N-[4-methyl-3-[2-[2-(2-morpholin-4-ylethylamino)pyrimidin-5-yl]ethynyl]phenyl]-3-morpholin-4-yl-benzamide

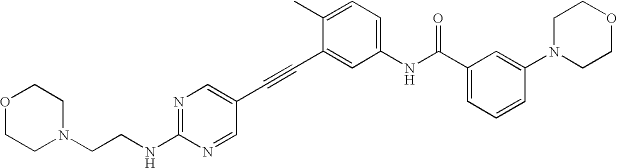

SM: [5-(5-Amino-2-methyl-phenylethynyl)-pyrimidin-2-yl]-(2-morpholin-4-yl-ethyl)-amine (Intermediate 40), 3-morpholin-4-ylbenzoic acid.

¹H NMR (DMSO-d₆) 2.40 (t, 6H), 3.19 (t, 4H), 3.28 (s, 3H under water), 3.43 (q, 2H), 3.56 (t, 4H), 3.76 (t, 4H), 7.11-7.19 (m, 1H), 7.26 (d, 1H), 7.34-7.39 (m, 2H), 7.44 (s, 1H), 7.49 (t, 1H), 7.64 (d, 1H), 7.91 (d, 1H), 8.44-8.51 (br s, 2H), 10.11 (s, 1H);

MS m/e MH⁺527.

### Intermediate 40

### [5-(5-Amino-2-methyl-phenylethynyl)-pyrimidin-2-yl]-(2-morpholin-4-yl-ethyl)-amine

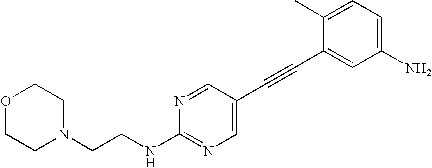

(5-Bromo-pyrimidin-2-yl)-(2-morpholin-4-yl-ethyl)-amine (Intermediate 32) (15 g), PdCl2(PPh3)2 (0.37 g), triphenylphosphine (0.7 g) and 3-ethynyl-4-methyl-phenyl amine (Intermediate 23) (8.2 mL) were added to piperidine (150 mL) and heated at reflux for 2 hours. The piperidine was then removed in vacuo to yield a yellow solid. Water (200 mL) was added, this was extracted with DCM (2×250 mL), dried (MgSO4) and solvent removed in vacuo to yield a yellow solid. The solid was dissolved in a minimum amount of hot DCM, and then diethyl ether added to precipitate a solid, cooling gave a yellow solid. Purification of the remaining liquor was achieved by flash chromatography on silica using 2.5-10% MeOH in DCM as eluent to give the title compound as a white solid (13.5 g, 77%);

1H NMR (300.132 MHz, CDCl3): 8.41 (s, 2H), 7.00 (d, 1H), 6.82 (s, 1H), 6.60 (d, 1H), 5.85 (brs, 1H), 3.72 (t, 4H), 3.60-3.50 (m, 4H), 2.61 (t, 2H), 2.50 (t, 4H), 2.36 (s, 3H);

MS m/e MH+338.

### Intermediate 41

### 5-Ethynyl-6-methyl-pyridin-3-ylamine

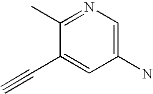

4-(5-Amino-2-methyl-pyridin-3-yl)-2-methyl-but-3-yn-2-ol (Intermediate 42) (assume quantitative yield from next step) was dissolved in toluene (300 ml), to this was added NaOH (5.6 g, 0.14 mol), the reaction was then refluxed for 5 hours after which the reaction was cooled and the solvent removed invacuo to yield a black tar. This was added to aqueous NaHCO3 (300 ml), extracted with diethyl ether (3×300 ml), dried (MgSO4) and solvent removed in vacuo to yield a brown solid. The residue was purified by column chromatography on silica eluting with 70-100% diethyl ether in iso-hexane to give the title compound as a off white solid (9.8 g, 80% over two steps); ¹H NMR (300 MHz, cdcl3) □ 7.97 (s, 1H), 7.05 (s, 1H), 3.59 (brs, 2H), 3.32 (s, 1H), 2.56 (s, 3H); MH⁺133

### Intermediate 42

### 4-(5-Amino-2-methyl-pyridin-3-yl)-2-methyl-but-3-yn-2-ol

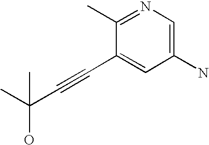

5-Iodo-6-methyl-pyridin-3-ylamine (Intermediate 43) (22 g, 93.6 mmol), PdCl2(PPh3)2 (0.7 g, 0.93 mmol), PPh3 (24 g, 93.6 mmol) and acetylenepropan-2-ol (17 ml, 0.18 mol) were added to piperidine (180 ml) and heated at reflux for 4 hours. The piperidine was removed in vacuo and the remaining residue was acidified with aqueous citric acid (300 ml) and extracted with diethyl ether (300 ml). The ether layer was re-extracted with aqueous citric acid (100 ml), the aqueous layers were combined and basified with potassium carbonate until pH12, this was then extracted with diethyl ether (3×300 ml), dried (MgSO4) and solvent removed invacuo to yield viscous black oil. This was then used in the next step without any purification.

### Intermediate 43

### 5-Iodo-6-methyl-pyridin-3-ylamine

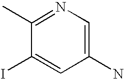

3-Iodo-2-methyl-5-nitro-pyridine (Intermediate 44) (33 g, 0.125 mol) and iron (21 g, 0,38 mol) were added to acetic acid (200 ml), the reaction was heated to 60 degrees for 2 hours. The acetic acid was removed in vacuo to yield a black sludge; this was treated with potassium carbonate in water (250 ml), until basic. The reaction was then extracted with diethyl ether (3×300 ml), dried (MgSO4) and solvent removed invacuo to yield a brown solid. Purification on silica eluting with 80-100% diethyl ether in iso-hexane gave the title compound as a white solid (25 g, 86%); ¹H NMR (300 MHz, cdcl3) □ 7.96 (s, 1H), 7.43 (s, 1H), 3.59 (brs, 2H), 2.62 (s, 3H).

### Intermediate 44

### 3-odo-2-methyl-5-nitro-pyridine

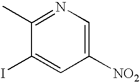

2-(3-Iodo-5-nitro-pyridin-2-yl)-malonic acid diethyl ester (Intermediate 45) (100 g) was added to 7.0N HCl (300 ml) and refluxed for 4 hours; the reaction was cooled and extracted with diethyl ether (3×200 ml). The aqueous was basified to pH10 with 10N NaOH, this was then re-extracted with diethyl ether (3×200 ml), dried (MgSO4) and solvent removed in vacuo to yield a black oil which solidified on standing. The solid was slurried in hot 30% ether/iso-hexane, the organic phase was then loaded onto a silica column, and the process was repeated until the entire solid had dissolved, the column was then eluted with 30% ether/iso-hexane to yield an orange solid. Recrystallisation from hot 50% ether/iso-hexane gave the title compound as a yellow solid. The process repeated until no solid crashed out (33 g, 60% over two steps); ¹H NMR (300 MHz, cdcl3) □ 9.26 (s, 1H), 8.82 (s, 1H), 2.87 (s, 3H); MS+H 265.

### Intermediate 45

### 2-(3-Iodo-5-nitro-pyridin-2-yl)-malonic acid diethyl ester

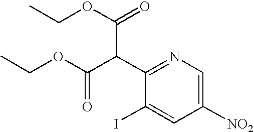

Sodium hydride (13.4 g, 0.28 mol) was added to DMF (500 ML), to this was slowly added diethylmalonate (45 ml, 0.28 mol), the reaction was stirred for 20 minutes before being cooled to 0oC., once cooled 2-chloro-3-iodo-5-nitropyridine (61 g, 0.21 mol) was slowly added. The reaction was stirred for 1 hour before being quenched with 2.0N HCl (300 ml), extracted with diethyl ether (3×300 ml), dried and solvent removed in vacuo to yield dark oil. This was used without any further purification.

1. A compound of the Formula I: 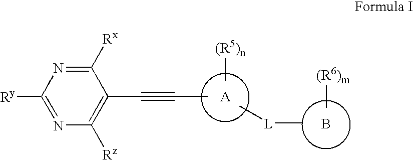
wherein one of R^{x} or R^{y} is a group NR¹R², and the other is a group R³ or R⁴, and R^{z} is a group R³ or R⁴,
R¹ and R² are independently selected from hydrogen, (1-6C)alkylsulfonyl, phenyl(CH₂)ᵤ— wherein u is 0, 1, 2, 3, 4, 5 or 6, (1-6C)alkanoyl, (1-6C)alkyl, (1-6C)alkoxycarbonyl, (3-6C)cycloalkyl(CH₂)ᵥ— in which v is 0, 1, 2, 3, 4, 5 or 6, or a 5 or 6 membered heteroaryl ring, or R¹ and R² together with the nitrogen atom to which they are attached represent a saturated or partially saturated 3 to 7 membered heterocyclic ring optionally containing another heteroatom selected from N or O;
wherein a (1-6C)alkyl, the (1-6C)alkoxy, the (1-6C)alkanoyl and the (3-6C)cycloalkyl groups are optionally substituted by one or more groups independently selected from fluoro, hydroxy, (1-6C)alkyl, (1-6C)alkoxy, (1-6C)alkoxy(1-6C)alkoxy, (1-6C)alkoxy(1-6C)alkoxy(1-6C)alkoxy, amino, mono(1-6C)alkylamino, di-[(1-6C)alkyl]amino, carbamoyl, mono(1-6C)alkylcarbamoyl, di-[(1-6C)alkyl]carbamoyl or —N(R^{d})C(O)(1-6C)alkyl, —N(R^{d})C(O)(1-6C)alkyl in which R^{d} is hydrogen or (1-6C)alkyl, or a saturated or partially saturated 3 to 7 membered heterocyclic ring, or a 5 or 6 membered heteroaryl ring;
wherein the (1-6C)alkyl, (1-6C)alkoxy, (1-6C)alkoxy(1-6C)alkoxy and (1-6C)alkoxy(1-6C)alkoxy(1-6C)alkoxy groups and the (1-6C)alkyl groups of the mono(1-6C)alkylamino, di-[(1-6C)alkyl]amino, mono(1-6C)alkylcarbamoyl, di-[(1-6C)alkyl]carbamoyl and/or —N(R^{d})C(O)(1-6C)alkyl groups are optionally substituted by one or more hydroxy groups;
wherein the phenyl is optionally substituted by one or more groups independently selected from halo, (1-6C)alkyl, (1-6C)alkoxy, amino, mono(1-6C)alkylamino or di-[(1-6C)alkyl]amino,di(1-6C)alkylamino, wherein a (1-6C)alkyl and the (1-6C)alkoxy groups are optionally substituted by one or more groups independently selected from hydroxy, amino, mono(1-6C)alkylamino or di-[(1-6C)alkyl]amino;di-(1-6C)alkylamino;
and wherein any heterocyclic and heteroaryl rings within R¹ and/or R² are optionally independently substituted by one or more of the following: (1-4C)alkyl, (1-4C)alkoxy, (1-4C)alkoxy(1-4C)alkyl, hydroxy, amino, mono(1-6C)alkylamino or di-[(1-6C)alkyl]amino,mono(1-6C)alkylamino, di-[(1-6C)alkyl]amino, a saturated or partially saturated 3 to 7 membered heterocyclic ring, or —C(O)(CH₂)_{z}Y wherein z is 0, 1, 2 or 3 and Y is selected from hydrogen, hydroxy, (1-4C)alkoxy,(1-4C)alkoxy, amino, mono(1-6C)alkylamino, di-[(1-6C)alkyl]amino or a saturated or partially saturated 3 to 7 membered heterocyclic ring;
and provided that when R¹ and/or R² is a (1C)alkanoyl group, then the (1C)alkanoyl is not substituted by fluoro or hydroxy;
R³ and R⁴ are independently selected from hydrogen, (1-6C)alkyl or (1-6C)alkoxy, (1-6C)alkoxy wherein the (1-6C)alkyl and the (1-6C)alkoxy groups are optionally substituted by one or more groups independently selected from: fluoro, hydroxy, (1-6C)alkyl, (1-6C)alkoxy, amino, mono(1-6C)alkylamino, di-[(1-6C)alkyl]amino,mono(1-6C)alkylamino, di(1-6C)alkylamino, carbamoyl, mono(1-6C)alkylcarbamoyl or di-[(1-6C)alkyl]carbamoyl, a saturated or partially saturated 3 to 7 membered heterocyclic ring or a 5 or 6 membered heteroaryl ring, wherein said heterocyclic and heteroaryl rings are optionally independently substituted by one or more of the following: (1-4C)alkyl, (1-4C)alkoxy, hydroxy, amino, mono(1-6C)alkylamino or di-[(1-6C)alkyl]aminodi(1-6C)alkylamino or a saturated or partially saturated 3 to 7 membered heterocyclic ring;
or one of R³ and R⁴ is as defined above and the other represents a group —NR¹R² as defined above.
A represents an aryl group or a 5 or 6 membered heteroaryl ring selected from furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl or 1,3,5-triazinyl;
R⁵ is selected from cyclopropyl, cyano, halo, (1-6C)alkoxy or (1-6C)alkyl, wherein the (1-6C)alkyl and the (1-6C)alkoxy groups are optionally substituted by cyano or by one or more fluoro;
n is 0, 1, 2 or 3;
L is attached meta or para on ring A with respect to the point of attachment of the ethynyl group and represents —N(R⁸)C(O)—(CR^{a}R^{b})ₓ-Z-(CR^{a}R^{b})_{y}—, —C(O)N(R⁹)—(CR^{a}R^{b})ₓ-Z-(CR^{a}R^{b})_{y}—, —C(R^{a})(R^{b})—N(R⁸)C(O)—(CR^{a}R^{b})ₓ-Z-(CR^{a}R^{b})_{y}— or —C(R^{a})(R^{b})—C(O)N(R⁹)—(CR^{a}R^{b})ₓ-Z-(CR^{a}R^{b})_{y}—
wherein Z is a direct bond, —O— or —N(R⁸)—
wherein x and y are independently 0, 1, 2 or 3, with the proviso that x+y <4,
wherein R⁸ and R⁹ independently represent hydrogen or (1-6C)alkyl,
wherein R^{a} and R^{b} independently represent hydrogen or (1-6C)alkyl or R^{a} and R^{b} together with the carbon atom to which they are attached represent (3-6C)cycloalkyl; and
wherein a (1-6C)alkyl group in R^{a} and R^{b} is optionally substituted by halo, cyano, hydroxy or a saturated or partially saturated 3 to 7 membered heterocyclic ring;
B represents a (3-7C)cycloalkyl ring, a saturated or partially saturated 3 to 7 membered heterocyclic ring, an aryl group,or a 5 or 6 membered heteroaryl ring,ring; or a 8, 9 or 10 membered bicyclic group which optionally contains 1, 2, 3 or 4 heteroatoms independently selected from N, O and S and which is saturated, partially saturated or aromatic;
and when B is a (3-7C)cycloalkyl ring, a saturated or partially saturated 3 to 7 membered heterocyclic ring or a saturated or partially saturated 8, 9 or 10 membered bicyclic group, the rings and the bicyclic group optionally bear 1 or 2 oxo or thioxo substituents;
R⁶ is selected from halo, cyano, oxo, a (3-7C)cycloalkyl ring, a saturated or partially saturated 3 to 7 membered heterocyclic ring, —S(O)ₚ-(1-6C)alkyl wherein p is 0, 1 or 2, —N(R^{c})C(O)(1-6C)alkyl ring and —N(R^{c})C(O)(1-6C)alkyl in which R^{c} is hydrogen or (1-6C)alkyl; or
R⁶ is selected from (1-6C)alkyl or (1-6C)alkoxy, (1-6C)alkyl, —S(O)ₚ-(1-6C)alkyl wherein p is 0, 1 or 2, or (1-6C)alkoxy,
wherein the (1-6C)alkyl, —S(O)ₚ-(1-6C)alkyl and the (1-6C)alkoxy(1-6C)alkoxy groups are optionally substituted by one or more groups independently selected from: cyano, fluoro, hydroxy, (1-6C)alkoxy,(1-6C)alkoxy, amino, mono(1-6C)alkylamino, di-[(1-6C)alkyl]amino, di(1-6C)alkylamino, a (3-7C)cycloalkyl ring or a saturated or partially saturated 3 to 7 membered heterocyclic ring; and
wherein the (3-7C)cycloalkyl ring and saturated or partially saturated 3 to 7 membered heterocyclic ring are optionally independently substituted by one or more groups selected from (1-6C)alkyl or hydroxy(1-6C)alkyl; and
m is 0, 1, 2 or 3;
or a salt or solvate thereof.
with the proviso that:
(i) L cannot be —C(R^{a})(R^{b})C(O)N(R⁹)—, —N(R⁸)C(O)C(R^{a})(R^{b})—, —N(R⁸)C(O)O— or —N(R⁸)C(O)N(R⁸)—.

2. A compound according to claim 1 wherein L is selected from N(R⁸)C(O)—, N(R⁸)C(O)—(CR^{a}R^{b})₂, —C(O)N(R⁹), —C(O)N(R⁹)—CR^{a}R^{b}), —C(R^{a})(R^{b})—N(R⁸)C(O)— or —C(R^{a})(R^{b})—C(O)N(R⁹)—(CR^{a}R^{b})—.

3. A compound according to claim 1 wherein R³ and R⁴ are other than a group NR¹R².

4. A compound according to claim 1 wherein R^{y} is a group NR¹R².

5. A compound according to claim 1 wherein R^{x} is a group NR¹R².

6. A compound according to claim 5 of the Formula IBi: 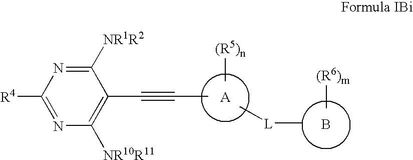
wherein:
R¹⁰ and R¹¹ are independently selected from hydrogen or (1-6C)alkyl; or a salt or solvate thereof.

7. A compound according to claim 1 wherein A is phenyl, pyridyl, thienyl or thiazolyl.

8. A compound according to claim 1 wherein B is isoxazolyl, pyrazolyl, tetrahydopyranyl, phenyl or benzodioxolyl.

9. A process for preparing a compound of formula I as defined in claim 1 or a salt or solvate thereof, which process comprises:
(a) for compounds of the formula I wherein L is —N(R⁸)C(O)—(CR^{a}R^{b})ₓ-Z-(CR^{a}R^{b})_{y}— or —C(R^{a}R^{b})N(R⁸)C(O)—(CR^{a}R^{b})ₓ-Z-(CR^{a}R^{b})_{y}—, the reaction of a compound of the formula II: 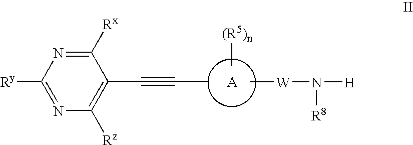
wherein W is —C(R^{a}R^{b})— or a direct bond and R^{x}, R^{y}, R^{z}, R⁵, R⁸, R^{a}, R^{b}, n, p and A have any of the meanings defined in claim 1 except that any functional group is protected if necessary, with a heterocycle of the formula III: 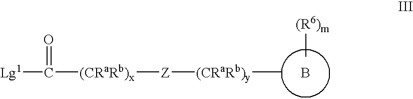
wherein Lg¹ is a displaceable group, and Z, R⁶, R^{a}, R^{b}, m, x, y and B have any of the meanings defined in claim 1 except that any functional group is protected if necessary; or or
(b) for compounds of the formula I wherein L is —C(R^{a}R^{b})C(O)N(R⁹)—(CR^{a}R^{b})ₓ-Z-(CR^{a}R^{b})_{y}—. or —C(O)N(R⁹)—(CR^{a}R^{b})ₓ-Z-(CR^{a}R^{b})_{y}—, the reaction of a compound of the formula IV: 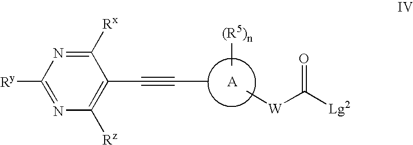
wherein Lg² is a displaceable group, W is —C(R^{a}R^{b})— or a direct bond and R^{x}, R^{y}, R^{z}, R⁵, R^{a}, R^{b}, n and A have any of the meanings defined in claim 1 except that any functional group is protected if necessary, with an amine of the formula V: 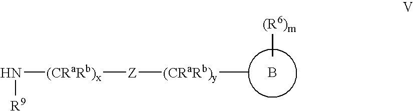
wherein R⁶, R⁹, R^{a}, R^{b}, m, x, y, B and Z have any of the meanings defined in claim 1 except that any functional group is protected if necessary; or
(c) for compounds of formula I wherein Z is —O— or —N(R^{a})— and when Z is —O— then x>0, the reaction of a compound of formula VI 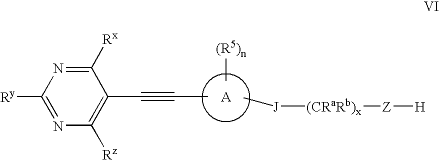
wherein J is selected from —N(R⁸)C(O)—, —C(O)N(R⁹)—, —C(R^{a})(R^{b})—N(R⁸)C(O)— or —C(R^{a})(R^{b})—C(O)N(R⁹)— and R^{x}, R^{y}, R^{z}, R⁵, R⁸, R⁹, R^{a}, R^{b}, x, n and A are as defined in claim 1 except that any functional group is protected if necessary and when Z is —O— then x>0, with a compound of formula VII, 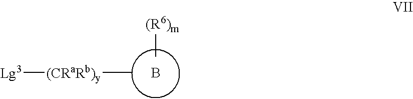
wherein Lg³ is a suitable displaceable group, and R^{a}, R^{b}, R⁶, y, m and B are as defined in claim 1 except that any functional group is protected if necessary;
(d) for compounds of formula I wherein Z is —O— or —N(R^{a})— and x>0, the reaction of a compound of formula VIII 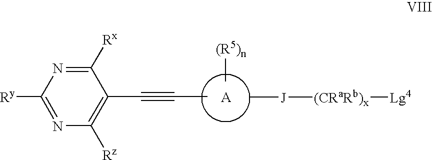
wherein Lg⁴ is a suitable displaceable group, J is selected from —N(R⁸)C(O)—, —C(O)N(R⁹)—, —C(R^{a})(R^{b})—N(R⁸)C(O)— or —C(R^{a})(R^{b})—C(O)N(R⁹)— and R^{x}, R^{y}, R^{z}, R⁵, R⁸, R⁹, R^{a}, R^{b}, n, and A are as defined in claim 1 except that any functional group is protected if necessary and x is 1, 2 or 3, with a compound of formula IX, 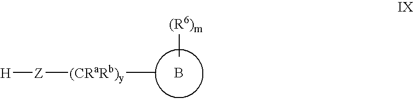
wherein Z, R^{a}, R^{b}, R⁶, y, m and B are as defined in claim 1 except that any functional group is protected if necessary; or
(e) for the preparation of a compound of formula IA, the reaction of a compound of the formula X: 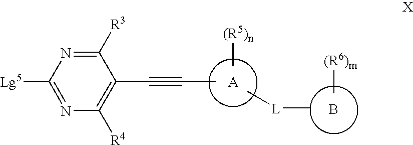
wherein Lg⁵ is a suitable displaceable group and R³, R⁴, R⁵, R⁶, n, m, A, B and L have any of the meanings defined in claim 1 except that any functional group is protected if necessary, with an amine of the formula HNR¹R², wherein R¹ and R² have any of the meanings defined hereinbefore except that any functional group is protected if necessary; or for the preparation of a compound of formula (IB), the reaction of a compound of the formula X′: 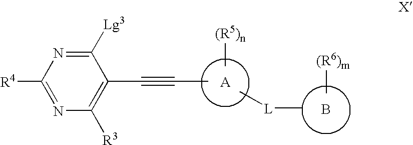
wherein Lg³ is a suitable displaceable group for example halogeno and R³, R⁴, R⁵, R⁶, n, m, A, B and L have any of the meanings defined in claim 1 except that any functional group is protected if necessary, with an amine of the formula HNR¹R², wherein R¹ and R² have any of the meanings defined hereinbefore except that any functional group is protected if necessary; or
(f) the reaction of a compound of the formula XI: 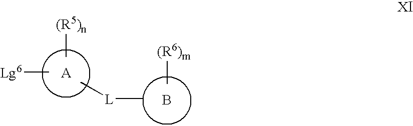
wherein Lg⁶ is a suitable displaceable group and R⁵, R⁶, n, m, A, B and L are as defined in claim 1 except that any functional group is protected if necessary, with an alkyne of the formula XII: 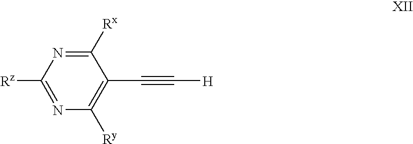
wherein R^{x}, R^{y}, and R^{z} are as defined in claim 1 except that any functional group is protected if necessary; or
(g) reaction of a compound of the formula XIII: 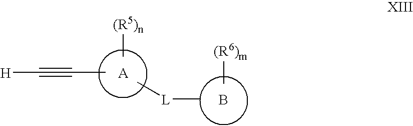
wherein R⁵, R⁶, n, m, A, B and L have any of the meanings defined in claim 1 except that any functional group is protected if necessary, with a pyrimidine of the formula XIV: 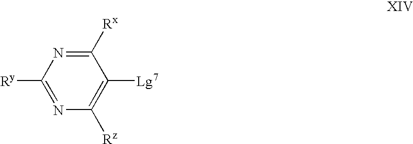
wherein Lg⁷ is a suitable displaceable group and R^{x}, R^{y} and R^{z} have any of the meanings defined in claim 1 except that any functional group is protected if necessary; or
and thereafter if necessary:
i) converting a compound of the Formula (I) into another compound of the Formula (I);
ii) removing any protecting groups;
iii) forming a salt or solvate.

10. A pharmaceutical composition which comprises a compound according to claim 1 or a pharmaceutically acceptable salt thereof, in association with a pharmaceutically-acceptable diluent or carrier.

11. (canceled)

12. (canceled)

13. (canceled)

14. (canceled)

15. A method of inhibiting Tie2 receptor tyrosine kinase in a warm-blooded animal, in need of such treatment, which comprises administering to said animal an effective amount of a compound according to claim 1, or a pharmaceutically acceptable salt thereof.

16. A method for producing an anti-angiogenic effect in a warm-blooded animal, in need of such treatment, which comprises administering to said animal an effective amount of a compound according to claim 1, or a pharmaceutically acceptable salt thereof.

17. A method of treating cancers in a warm-blooded animal, in need of such treatment, which comprises administering to said animal an effective amount of a compound according to claim 1, or a pharmaceutically acceptable salt thereof.
